(19) United States
(12) Patent Application Publication
(10) Pub. No.: US 2023/0266328 A1
(43) Pub. Date: Aug. 24, 2023

(54) MULTIOMIC ANALYSIS OF NANOPARTICLE-CORONAS

(71) Applicant: THE UNIVERSITY OF MANCHESTER, Manchester (GB)

(72) Inventors: Marilena HADJIDEMETRIOU, Manchester (GB); Kostas KOSTARELOS, Manchester (GB); Lois GARDNER, Manchester (GB); Lana PAPAFILIPPOU, Manchester (GB)

(21) Appl. No.: 18/041,131

(22) PCT Filed: Aug. 9, 2021
(86) PCT No.: PCT/GB2021/052056

§ 371 Date: Feb. 9, 2023

(30) Foreign Application Priority Data
Aug. 10, 2020 (GB) 2012434.3

(51) Int. Cl. G01N 33/574; C12Q 1/6886; G01N 33/543; G01N 33/68; G01N 33/92

(52) U.S. Cl. CPC G01N 33/57449; C12Q 1/6886; G01N 33/54346; G01N 33/5432; G01N 33/6848; G01N 33/92; C12Q 2600/156; C12Q 1/6851

(57) ABSTRACT

The present invention relates to methods for simultaneously identifying and/or detecting distinct classes of biomarker in biofluid samples, such as blood.

### FIELD OF THE INVENTION

The present invention relates to methods for simultaneously identifying and/or detecting distinct classes of biomarker in biofluid samples, such as blood. Such method may be useful in analysing disease specific biomarkers. The method creates a nanoparticle-based liquid biopsy platform that simultaneously harvests multiple classes/families of molecules (including proteins, nucleic acids, and lipids) from a single biofluid sample and then analyzes these classes of molecules. Suitably, the biofluid is from a subject with or suspected of having a disease and the biomolecules analyzed are disease-specific biomarkers. In particular, the methods involve contacting nanoparticles with a biofluid from a subject, optionally in a diseased state, and subsequent multi-omic analysis of the biomolecule corona formed on said nanoparticles. In addition, the present invention relates to methods for monitoring cancer progression in a subject by assessing the type and/or amount of tumour-specific biomarkers from two or more classes simultaneously as measured over time.

### INTRODUCTION

A biomarker, or biological marker, generally refers to a qualitative and/or quantitative measurable indicator of some biological state or condition. Biomarkers are typically molecules, biological species or biological events that can be used for the detection, diagnosis, prognosis and prediction of therapeutic response of diseases.

Ongoing efforts are focused on the development of robust and high-throughput ‘omics’ platforms for the discovery of minimally invasive molecular biomarkers to aid early and accurate cancer diagnosis, monitor tumour growth and response to therapies. Despite tremendous efforts and investment by major stakeholders, only few protein cancer biomarkers have been validated and received FDA approval, raising concerns regarding the efficiency of the biomarker-development pipeline, and of the FDA-approved biomarkers, the majority are used to monitor the progression of cancer, rather than enabling its early diagnosis.

Proteins are the biological endpoints that govern most pathophysiological processes and they and the nucleic acid that encode them have therefore attracted most interest so far as biomarkers for cancer diagnostics. Blood is the most valuable repertoire of cancer biomarkers; however, the discovery of tumour-derived protein signatures directly from blood is hindered by the wide concentration range of blood proteins, in addition to the preponderance of highly abundant proteins. The same challenge is faced with the detection of tumour-derived nucleic acid signatures.

Over the last decade, biomedical applications of nanoparticles (NPs) have been challenged due to the spontaneous adsorption of biomolecules onto their surface upon incubation with complex biofluids, known as the ‘protein’ or ‘biomolecule corona’.¹ The bio-nanotechnology field has since invested considerable resources investigating the corona composition in an attempt to prevent NP-protein interactions and consequently limit opsonisation-mediated clearance from blood and masking of surface ligands.²⁻⁶ Protein corona formation is now a widely accepted phenomenon and has been documented for a wide range of NPs, including lipid-, metal-, polymer- and carbon-based nanomaterials, with their composition and surface chemistry altering the specific classes of proteins adsorbed.⁶

Biomolecule corona formation has become a popular line of research the last decade and ongoing research is mainly focused on the proteomic analysis of corona profiles after the ex vivo and more recently the in vivo interaction of NPs with biofluids (mainly plasma). Our laboratory has illustrated the potential exploitation of protein corona as a proteomic biomarker discovery platform that enables a higher-definition, in-depth analysis of the blood proteome and the enrichment of low abundant disease-specific molecules (see WO2018/046542 and^{8-16,13). The surface-capture of a complex blood proteome by NPs has sparked interest in utilizing the biomolecule corona fingerprinting as a proteomic discovery platform. Nanoparticle-protein interactions at the bio-nano interface not only can shed new light on the development of nanotechnologies but are now gradually being exploited as an engineering tool with therapeutic and diagnostic capabilities.}

Research into cell free nucleic acid biomarker detection has been carried out but so far has failed to provide suitable methods to accurately identify/discover and detect biomarkers. One particular problem is that currently available laboratory tests detect only a minute fraction of potential biomarkers, due to their extremely low concentration in biofluids. In addition to the ‘swamping’ effect, caused by other “non-specific” high abundant molecules, this causes significant difficulties. Furthermore, such methods are mainly used to detect already known disease-specific nucleic acid molecules (such as activating mutations associated with cancer).

Despite recent advances in analyzing the blood-circulating genome, very little attention has been placed on the utilization of the spontaneous interaction of NPs with nucleic acids upon incubation with biological fluids.

Surprisingly, the inventors have found that the biomolecule corona formed on nanoparticles after following methods involving administration of nanoparticles to a subject in a diseased state or incubation of nanoparticles in a biofluid sample taken from a subject in a diseased state results in interaction of the nanoparticles with cell free nucleic acid biomolecules as well as lipid and protein biomarkers.

The novel methods take advantage of the interaction of nanoparticles with distinct classes of biomolecules (e.g. protein, lipid, nucleic acid) which can then be analyzed simultaneously (including in parallel) as a way to detect and monitor disease and also to facilitate the detection of previously unknown disease-specific biomolecules.

### SUMMARY OF THE INVENTION

The present study includes experimental evidence that cfNA exists in the biomolecule corona formed around NPs in human plasma, and at quantifiable levels. The ability of NPs to form coronas that include nucleic acid as well as other classes of biomolecule, such as lipids, metabolites and proteins and to detect/analyze these simultaneously as part of a multi-omic analysis is new.

According to a first aspect of the invention there is provided a method of identifying biomarkers from two or more distinct biomolecule classes in a biofluid, wherein the method comprises:
(a) contacting a plurality of nanoparticles with a biofluid to allow a biomolecule corona to form on the surface of said nanoparticles;
(b) isolating the nanoparticles and surface-bound biomolecule corona; and
(c) analyzing the biomolecule corona to identify biomarkers from two or more distinct biomarker classes.

In particular embodiments, step (a) is performed in vivo by administering a plurality of nanoparticles to a subject, such as by intravenous injection, or step (a) is performed in vitro (e.g. ex vivo) using a biofluid sample that has been taken from the subject.

Suitably the biomolecule corona is analyzed by two or more of proteomic, genomic and lipidomic analysis. Suitably, the analysis by two or more of proteomic, genomic and lipidomic analysis is conducted on a single biofluid sample. Suitably the analysis of each biomolecule class is conducted simultaneously or separately.

The method of the first aspect of the invention may be used to identify new biomarkers.

The methods result in an interaction between the nanoparticles and a greater number of different types of biomolecules, in particular proteins, than can be detected by direct analysis of biofluids taken from a subject, such as one in a diseased state. It is to be understood that the method involves identification of a biomarker that provides a measurable indicator of some biological state or condition. This includes, but is not limited to, the discovery of unique disease-specific biomolecules (those biomolecules that are only present in a diseased state) but also includes detection of changes (for example, a statistically significant change) in biomolecule(s) that are present in both healthy and diseased states, for example upregulation or down regulation of biomolecules in a diseased state when compared to the healthy state or at a different time point. It will be understood that in order to identify a potential disease-specific biomarker, comparison against a suitable non-diseased control reference can be required.

By up-regulation or down-regulation of a particular biomolecule we mean an increase or decrease, respectively, in the amount and/or abundance of the biomarker.

In particular embodiments, the biomolecule level is reduced or down-regulated to less than 90%, such as less than 80% such as less than 70% for example less than 60%, for example less than 50%, such as less than 40%, such as less than 30% such as less than 20% for example less than 10%, for example less than 5%, such as completely inhibited (0%) compared to the control level.

In particular embodiments, the biomolecule level is increased or up-regulated to more than 110%, such as more than 120% such as more than 130% for example more than 150%, for example more than 175%, such as more than 200%, such as more than 250% such as more than 300% for example more than 350% of the control amount.

In one particular embodiment, the methods involve identifying panels of biomarkers (multiplexing), which can lead to increased sensitivity and specificity of detection.

In a further particular embodiment, the methods facilitate the detection of previously unknown unique disease-specific biomolecules. In a particular embodiment, the unknown biomarkers are unique biomolecules, meaning that the biomolecules that would not have been detected if analysis was carried out directly on biofluid, such as plasma, isolated from the subject.

In yet a further particular embodiment, the methods allow identification or detection of a biomarker without the need for invasive tissue sampling, e.g. a biopsy.

The methods are applicable to a wide range of nanoparticles and allow the benefit of removal of unbound and highly abundant biomolecules to allow identification of low abundant biomarkers, in particular proteins, that would otherwise be undetected. In addition to identification of potential biomarkers, the methods can also be employed to monitor changes in biomarkers, for example in response to therapy and/or to assist in diagnosis.

Suitably, the method can be used to detect or monitor a disease in a subject. The methods disclosed herein are applicable to any disease state in which detection and/or monitoring of biomarkers would be beneficial. Furthermore, particular methods of the invention, which can be employed to distinguish between healthy and diseased states in a subject, are applicable to a wide range of diseases, including but not limited to, cancer and neurodegenerative diseases. In particular, the methods of the invention can be used to diagnose a disease, such as cancer, including in the early detection of a diseased state such as the presence of a cancer or pre-cancerous condition in a human subject. The methods of the invention can also be employed to discover novel biomarkers and biomarker fingerprints.

According to a second aspect of the invention there is provided a method for detecting a disease state in a subject, comprising:

(a) contacting a biofluid sample from the subject with a plurality of nanoparticles under conditions to allow a biomolecule corona to form on the surface of said nanoparticles; and

(b) analyzing the biomolecule corona for one or more disease-specific biomarkers from two or more biomolecule classes, which is determinative of the presence of a disease in said subject.

In a particular embodiment, the disease is cancer.

The method can be used to monitor disease progression, for example to monitor the efficacy of a therapeutic intervention. Suitably the disease is cancer. Suitable cancers include ovarian, lung, prostate, melanoma and blood cancer, including leukemia, lymphoma and myeloma. In a particular embodiment, the cancer is ovarian cancer.

According to a third aspect of the invention there is provided a method for monitoring cancer progression in a subject, comprising:

(a) contacting a biofluid sample from the subject with a plurality of nanoparticles under conditions to allow a biomolecule corona to form on the surface of said nanoparticles; and

(b) analyzing the biomolecule corona for one or more cancer-specific biomarkers from two or more biomolecule classes;

wherein the degree of cancer progression is determined based on the level of the cancer-specific biomarker(s) relative to a reference amount.

Suitably, in any of the aspects of the invention, the biofluid is blood, plasma, urine, saliva, lacrimal, cerebrospinal and ocular fluids, or any combination thereof. Suitably, the biofluid is a blood or blood fraction sample, such as serum or plasma. Suitably, the blood or blood fraction sample is from circulating blood.

In particular embodiments of any of the aspects of the invention, the biomolecule corona is analyzed by two or more of proteomic, genomic and lipidomic analysis.

The methods of any of the aspects of the invention may offer high sensitivity and a high level of precision which allows for the identification, detection and/or quantification of disease biomarkers and/or the abundance thereof, even when present in low abundance, which otherwise may be very difficult to identify.

Any embodiment described herein can be applied to any aspect of the invention unless indicated otherwise or it is apparent to the person of skill in the art that such embodiment cannot apply.

Accession numbers herein detailed are based on the SwissProt_2016_04 database.

### DESCRIPTION OF THE DRAWINGS

In order that the invention may be more clearly understood one or more embodiments thereof will now be described, by way of example only, in relation to an experimental study and with reference to the accompanying drawings, of which:

FIG. 1—Schematic representation of sample pre-processing and cfDNA quantification method pipelines. A) Schematic overview of human plasma and liposomal nanoparticle (NP) incubation and subsequent size-exclusion purification methodology. B) Method analysis pipeline for plasma processing (including cfDNA purification) and subsequent q-PCR quantification of cfDNA in NP corona samples and plasma control samples.

FIG. 2—Characterisation of cfDNA content in the healthy ex vivo biomolecule corona. A) cfDNA and liposomal lipid quantification across 15 chromatographic fractions. The purified cfDNA from a single healthy pooled plasma sample incubated with and without liposomal nanoparticles (NPs) was quantified by a highly-sensitive LINE-1 real-time PCR assay. NPs and cfDNA are expressed as percentage (%) of total recovered across chromatographic fractions. B) RNase P real-time cfDNA quantification of pooled ex vivo NP⁽⁺⁾ corona samples and NP⁽⁻⁾ controls (size-purified plasma). cfDNA was measured directly and in samples with additional cfDNA purification step. C) cfDNA concentrations in NP⁽⁺⁾ corona samples and NP⁽⁻⁾ controls were confirmed using the LINE-1 real-time PCR assay. For graphs B and C cfDNA is expressed as percentage recovery (%) relative to QIAGEN's QIAamp® Circulating Nucleic Acid extraction kit (average of three replicates). All error bars represent mean and standard deviation. Groups were compared using a student t-test (p values <0.05 were considered significant).

FIG. 3—Assessing the accuracy of direct real-time PCR cfDNA quantification in ex vivo healthy and disease nanoparticle corona samples. A) RNase P real-time qPCR quantification of in pooled healthy liposomal corona samples and liposome⁽⁻⁾ plasma controls. B) Direct RNase P qPCR inhibition determined using 2-fold dilution of pooled NP corona samples. C-D) LINE-1 real-time qPCR quantification of cfDNA in late-stage serous ovarian cancer ex vivo biomolecule corona samples (n=8). Graph C represents cfDNA in NP corona samples and NP corona purified cfDNA, whereas graph D represents cfDNA in unpurified plasma (diluted 1:40) and purified plasma. All error bars represent mean and standard deviation. Groups were compared using a student t-test was performed (adjusted p values <0.05 were considered significant). E) Clinical details of eight late-stage ovarian cancer plasma samples included in graphs C and D.

FIG. 4—Reproducibility & linearity experiments of healthy plasma NP corona samples. A) Reproducibility data showing the percentage recovery (%) of QIAamp® purified NP corona cfDNA across liposome NP batches relative to QIAamp extracted plasma cfDNA (100%). B-C) Linearity data to investigate the effect of liposome concentration and plasma volume on cfDNA content in the liposome biomolecule corona. B) Graph highlighting the effect of plasma volume on cfDNA concentration (ng cfDNA/sample). Standard protocol 820 μL plasma: 180 μL liposomes. C) Graph showing the effect of liposome concentration on cfDNA concentration (ng cfDNA/sample). 12.5 mM liposomes represent standard protocol. All error bars represent mean and standard deviation. Three groups or more were compared using a one-way analyzes of variance (ANOVA) test followed by the Tukey's multiple comparison test. Adjusted p values <0.05 were considered significant.

FIG. 5—Cell-free DNA (cfDNA) detection in the ex vivo ovarian cancer biomolecule corona. A) Normalised cfDNA concentration (ng/μM lipid) in corona-coated liposomes (ovarian cancer samples and age- and sex-matched healthy controls), measured using a highly-sensitive LINE-1 real-time PCR assay and robust inhibitor-resistant polyermase. B) The same data with ovarian cancer patients separated into early stage (1 & 2) and late-stage (3 & 4) cancers. All error bars represent mean and standard deviation. Three groups or more were compared using a one-way analyzes of variance (ANOVA) test followed by the Tukey's multiple comparison test. For comparisons of two groups a student t-test was performed (adjusted p values <0.05 were considered significant).

FIG. 6—Histone proteins identified by LC-MS/MS in the biomolecule corona of healthy and ovarian cancer female plasma samples. A) LC-MS/MS normalised protein abundance of histones H2A, H2B and H4 in ovarian cancer corona samples and age-matched healthy corona controls. A one-way ANOVA was performed by the Progensis QI software with significance bars representing FDR-adjusted p values. B) Table summarising the relative abundance of proteins identified by LC-MS/MS associated with nucleosomes (DNA-histone complex) known to contain cfDNA. Max fold change between ovarian cancer corona samples and healthy corona controls is provided with FDR-adjusted p value from a one-way ANOVA in Progensis QI).

FIG. 7—Physiochemical characterisation of liposome nanoparticles (NPs). A) Graphs representing the size (diameter in nm) and zeta-potential distribution (mV) of PEG:HSPC:CHOL liposome batches 1-3. B) Table listing the mean average size (nm), polydispersity index (PDI) and zeta-potential (mV) of each liposome batch including standard deviations.

FIG. 8— Characterisation of protein, cfDNA and lipid content of the biomolecule corona. A) Schematic overview of biofluid nanoparticle incubation and size-based purification methodology. B) Negative TEM staining imaging of purified plasma controls and corona-coated nanoparticles, recovered post-incubation with human plasma obtained from healthy donors. All scale bars are 100 nm. C) Method analysis pipeline for plasma processing and subsequent quantification of proteins, nucleic acids and lipids in nanoparticle corona samples and plasma control samples.

FIG. 9— Proteomic Analysis of the nanoparticle biomolecule corona. (A) Imperial stained SDS-PAGE gels of i) purified human plasma controls and ii) corona proteins associated with liposomes post-incubation with plasma obtained from healthy donors after a two-step purification protocol; (B) Comparison between the total amount of protein i) identified in purified human plasma controls (n=3) and ii) adsorbed onto liposomes after their ex vivo incubation with plasma obtained from healthy donors (n=3) after a two-step purification protocol, (expressed as μg/mL). Protein concentration values represent the average and standard error. * indicates p<0.05 (p=0.0175); (C) Top 20 most abundant proteins found onto the surface of nanoparticles, as these identified by LC-MS/MS; (D) Classification of all identified proteins according to their molecular weight (kDa).

FIG. 10— Characterisation of cfDNA content in the iomolecule corona. A) cfDNA and liposomal lipid quantification across 15 chromatographic fractions. The purified cfDNA from healthy pooled plasma incubated with and without liposomal nanoparticles (NPs) was quantified by a sensitive LINE-1 qPCR assay. Nanoparticles and cfDNA are expressed as percentage (%) of total recovered across chromatographic fractions. B) RNase P qPCR cfDNA quantification in pooled ex vivo NP corona samples and NP⁽⁻⁾ controls (size-purified plasma). cfDNA was measured directly and in samples with additional cfDNA purification step. C) Subsequent cfDNA quantification using a sensitive LINE-1 qPCR with inhibitor resistant polymerase. cfDNA in graphs B and C is expressed as percentage recovery (%) relative to a standard total circulating nucleic acid extraction kit (Qiagen). All error bars represent mean and standard deviation. Three groups or more were compared using a one-way analyses of variance (ANOVA) test followed by the Tukey's multiple comparison test. For comparisons of two groups a student t-test was performed (adjusted p values <0.05 were considered significant).

FIG. 11—Lipidomic Analysis of the nanoparticle-biomolecule corona. (A) Quantification of complex lipids found in i) bare HSPC:CHOL liposomes and ii) corona-coated liposomes, expressed in ng per 30 μL of extracted sample. Complex lipids identified include DG: Diacylglycerols; TG: Triacylglycerols; FFA: Free Fatty Acids; PC: Phosphatidylcholines; LPC: Lysophosphatidylcholines; PE: Phosphatidylethanolamines; SM: Sphingomyelins; (B) Quantification of ceramides and endocannabinoids found in i) bare HSPC:CHOL liposomes and ii) corona-coated liposomes, expressed in ng per 50 μL of extracted sample; (C) Quantification of oxylipins found in i) bare HSPC:CHOL liposomes and ii) corona-coated liposomes, expressed in ng per 1 mL of extracted sample.

FIG. 12—Multi-omics analysis of the biomolecule corona for biomarker discovery. Proteomic and genomic comparison of the biomolecule coronas formed in plasma samples obtained from ovarian carcinoma patients and healthy controls. Volcano plots represent the potential protein biomarkers differentially abundant between: A) healthy controls and early stage ovarian cancer patients; B) healthy controls and late stage ovarian cancer patients and C) early stage and late stage ovarian cancer patients. D) Total cfDNA quantification (LINE-1 qPCR cfDNA (ng/μM lipid)) in corona-coated liposomes (ovarian cancer samples and age- and sex-matched healthy controls). Groups were compared using a one-way analyses of variance (ANOVA) test followed by the Tukey's multiple comparison test (adjusted p values <0.05 were considered significant). E) Quantitative PCR (qPCR) detection of miR-200 family microRNAs (miRNAs) in the ex vivo late-stage serous ovarian cancer corona. Graphs represent miRNA-200c and miR-141 qPCR expression, with individual patient samples connected to observe patient-specific enrichment patterns. All error bars represent mean and standard deviation. Three groups or more were compared using a one-way analyses of variance (ANOVA) test followed by the Tukey's multiple comparison test. Adjusted p values <0.05 were considered significant.

### DETAILED DESCRIPTION OF THE INVENTION

The practice of particular embodiments of the invention will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See, e.g., Sambrook, et al., Molecular Cloning: A Laboratory Manual (3rd Edition, 2001); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience.

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred embodiments of compositions, methods and materials are described herein.

### Definitions

The articles “a,” “an,” and “the” are used herein to refer to one or to more than one (i.e. to at least one) of the grammatical object of the article.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

The term “biomolecule” includes, but is not limited to, proteins, peptides, fatty acids, lipids, amino acids, amides, sugars and nucleic acids (such as for example different types of DNA or RNA).

As used herein, the term “disease-specific biomarker” refers to a biomarker which is associated with or indicative of a disease. Examples of certain cancer-specific biomarkers include: mutations in genes of KRAS, p53, EGFR or erbB2 for colorectal, esophageal, liver, and pancreatic cancer; mutations in BRCA1 and BRCA2 genes for breast and ovarian cancer; and, abnormal methylation of tumor suppressor genes p16, CDKN2B, and p14ARF for brain cancer. As used herein, the term “high-throughput sequencing” is also referred to as “second-generation sequencing,” and the principles of high-throughput sequencing techniques are well known to those of skill in the art, and high-throughput sequencing is typically performed on microporous chips. High throughput sequencing techniques and the reagents and devices used therein are conventional in the art. Commercially available high throughput sequencing chips and reagents are readily available, for example, from Life Technologies Inc. To conduct high throughput sequencing the cfDNA captured in the corona may need a pre-treatment process such as amplification, end-repair, ligation, labeling and/or purification, etc. in order to construct a cfDNA library prior to high-throughput sequencing, and the techniques required for this are understood by those of skill in the art of high-throughput sequencing, and can be constructed, for example, using the NEBNext Fast DNA Fragmentation & Library Prep Set for Ion Torrent (Life Technologies Cat. No. 4474180) kit.

As used herein, the term “in vitro” means performed or taking place in a test tube, culture dish, or elsewhere outside a living organism. The term also includes ex vivo because the analysis takes place outside an organism.

As used herein, the term “isolated” means material that is substantially or essentially free from components that normally accompany it in its native state. In particular embodiments, the term “obtained” or “derived” is used synonymously with isolated.

Multi-omics is a biological analysis approach in which the data sets are multiple “omes”, such as the genome, proteome, transcriptome, epigenome, lipidome and metabolome. For a review on multi-omics see Hasin et al. Genome Biology. “Multi-omics approaches to disease”. 18(83), 2017; https://doi.org/10.1186/s13059-017-1215-1.

As used herein “multi-omics” means analysis that generates data at two or more biological levels including at the genome, epigenome, transcriptome, proteome, and metabolome level. As used herein, “multi-omic analysis” refers to two or more types of analysis selected from: nucleic acid, protein and lipid analysis.

Genomics is an area within genetics that concerns the sequencing and analysis of an organism's genome. The genome is the entire DNA content that is present within one cell of an organism.

As used herein, “genomics” is the analysis of genes and nucleic acids generally (including DNA and RNA), and includes transcriptomics (the study of RNA generally and in particular RNA transcripts).

As used herein, “proteomics” is the analysis of proteins and elements of protein (referred to herein as a protein element or protein derivative) such as peptides (short chains of amino acids, e.g. 2-10 amino acids) and polypeptides (longer chains of amino acids).

Lipidomics is the large-scale study of pathways and networks of cellular lipids in a biological system. The term “lipidome” is often used to describe the complete lipid profile within a cell, tissue, organism, or ecosystem and is a subset of the term “metabolome” which also includes the three other major classes of biological molecules: proteins/amino-acids, sugars and nucleic acids.

As used herein, “lipidomics” is the analysis of lipids and elements of lipids. The metabolome is typically defined as the complete complement of all small molecule metabolites (<1500 Da), such as metabolic intermediates, hormones and other signaling molecules, and secondary metabolites, found in a specific cell, organ or organism (Wishart DS Human metabolome database: completing the ‘human parts list’. Pharmacogenomics 8:683-686, 2007). Metabolomics is the scientific study of chemical processes involving metabolites, the small molecule substrates, intermediates and products of metabolism.

A “target genetic locus” or “nucleic acid target region” refers to a region of interest within a nucleic acid sequence. In various embodiments, targeted genetic analyzes are performed on the target genetic locus. In particular embodiments, the nucleic acid target region is a region of a gene that is associated with a particular genetic state, genetic condition, genetic diseases; genetic mosaicism, predicting response to drug treatment; diagnosing or monitoring a medical condition; microbiome profiling; pathogen screening; or organ transplant monitoring.

As used herein “targeted genetic analyzes” refers to investigations of specific known genetic regions, including mutations, for example those that are known to be associated with a disease. Exemplary genetic regions include genes (e.g. any region of DNA encoding a functional product) or a part thereof, gene products (e.g., RNA and expression of genes). The genetic regions can include variations with the sequence or copy number. Exemplary variations include, but are not limited to, a single nucleotide polymorphism, a deletion, an insertion, an inversion, a genetic rearrangement, a copy number variation, or a combination thereof. The methods of the invention can be used to isolate cfNA that can then be subjected to any desired targeted genetic analysis.

As used herein, the terms “circulating NA,” “circulating cell-free NA” and “cell-free NA” are often used interchangeably and refer to nucleic acid that is extracellular DNA or RNA, DNA or RNA that has been extruded from cells, or DNA or RNA that has been released from lysed, necrotic or apoptotic cells.

A “subject,” “individual,” or “patient” as used herein, includes any animal that exhibits a symptom of a condition that can be detected or identified with compositions contemplated herein. Suitable subjects include laboratory animals (such as mouse, rat, rabbit, or guinea pig), farm animals (such as horses, cows, sheep, pigs), and domestic animals or pets (such as a cat or dog). In particular embodiments, the subject is a mammal. In certain embodiments, the subject is a non-human primate and, in a particular embodiment, the subject is a human.

A major limitation of classical omic studies is the analysis at only one level of biological complexity. For example, transcriptomic studies will provide information at the transcript level, but many different entities contribute to the biological state of the sample (genomic variants, post-translational modifications, lipid products, metabolic products, interacting organisms, among others). With the advent of high-throughput biology, it is becoming increasingly affordable to make multiple measurements, allowing transdomain (e.g. RNA and protein levels) correlations and inferences. These correlations aid the construction or more complete biological networks, filling gaps in our knowledge.

It is therefore desirable to identify platforms systems that facilitate multi-omic analysis.

Methods of the Invention

According to a first aspect of the invention there is provided a method of identifying biomarkers from two or more distinct biomolecule classes in a biofluid, wherein the method comprises:
(a) contacting a plurality of nanoparticles with a biofluid to allow a biomolecule corona to form on the surface of said nanoparticles;
(b) isolating the nanoparticles and surface-bound biomolecule corona; and
(c) analyzing the biomolecule corona to identify biomarkers from two or more distinct biomarker classes.

Advantageously, the method according to the first aspect is used to identify biomarkers from two or more distinct biomolecule classes. It is to be understood that the term “identify” in this context relates to discovering biomarkers which are new (i.e., previously not known and/or previously not associated with a particular disease or stage of disease that the subject from which the biofluid was taken has).

In one embodiment, there is provided the method according to the first aspect wherein the method identifies biomarkers from two or more distinct biomolecule classes in a biofluid from a subject in a diseased state wherein the biomarkers have previously not associated with a particular disease or stage of disease.

In one embodiment of the first aspect of the invention, there is provided a method of identifying biomarkers from two or more distinct biomolecule classes in a biofluid, wherein the method comprises:
(a) contacting a plurality of nanoparticles with a biofluid to allow a biomolecule corona to form on the surface of said nanoparticles;
(b) isolating the nanoparticles and surface-bound biomolecule corona; and
(c) analyzing the biomolecule corona to identify biomarkers from two or more distinct biomarker classes wherein the biomolecule corona is analyzed by two or more of proteomic, genomic and lipidomic analysis.

In particular embodiments, step (a) is performed in vivo by administering a plurality of nanoparticles to a subject or in vitro/ex vivo using a biofluid sample that has been taken from the subject.

In a particular embodiment, step (a) is performed in vivo by administering a plurality of nanoparticles to a subject, a biofluid sample is then taken from the subject and analyzed. Prior to analysis, the particles are isolated from the biofluid and purified to remove unbound and highly abundant biomolecules. In one embodiment the nanoparticles are administered to the subject by intravenous injection.

According to a variation of the first aspect of the invention there is provided a method of identifying biomarkers from two or more distinct biomolecule classes in a biofluid, wherein the method comprises:
(a) administering a plurality of nanoparticles to a subject to allow a biomolecule corona to form on the surface of said nanoparticles;
(b) isolating the nanoparticles and surface-bound biomolecule corona; and
(c) analyzing the biomolecule corona to identify biomarkers from two or more distinct biomarker classes.

In this approach, step (a) of the method involves administering a plurality of nanoparticles to a subject to allow a biomolecule corona to form on the surface of said nanoparticles. Suitably, administration can be by any route that allows the biomolecule corona to form. Suitable routes of administration include but are not limited to intravenous, oral, intracerebral (including spinal), intraperitoneal and intra-occular. Conveniently, the route of administration is by intravenous injection. The biomolecule corona typically forms within less than 10 minutes from administration. Suitably, the subject is suffering from a disease (is in a diseased state).

A biofluid sample comprising some of the introduced nanoparticles is then extracted from the subject; for example, by taking a blood sample. In a particular embodiment, the nanoparticles are isolated from the biofluid sample prior to analysis. Any isolation technique that is capable of preserving the surface-bound biomolecule corona is suitable. Conveniently, the nanoparticles with surface-bound biomolecule corona are isolated from the biofluid and purified to remove unbound and highly abundant biomolecules (for example albumin and/or immunoglobulins, which can constitute 90% of the plasma proteome) to allow identification of lower abundant biomarkers. The method therefore allows minimization of any masking caused by the highly abundant proteins. Conveniently, the isolation is achieved by a method comprising size exclusion chromatography followed by ultrafiltration.

According to another variation of the first aspect of the invention there is provided a method of identifying biomarkers from two or more distinct biomolecule classes in a biofluid, wherein the method comprises:
(a) incubating a plurality of nanoparticles in a biofluid sample taken from a subject to allow a biomolecule corona to form on the surface of said nanoparticles.
(b) isolating the nanoparticles and surface-bound biomolecule corona; and
(c) analyzing the biomolecule corona to identify biomarkers from two or more distinct biomarker classes.

In particular embodiments of this aspect of the invention, in step (c) at least one of the biomarker classes is selected from the group consisting of: protein, nucleic acid and lipid, or any complexes of these (such as nucleic acid/protein complex).

Suitably, such incubation can be carried out ex vivo or in vitro (herein the term in vitro includes ex vivo). In this approach, the NP corona is formed in vitro by incubating the plurality of nanoparticles in a biofluid sample to be analyzed. Conveniently, this involves incubating at a suitable temperature, such as at about 37° C., for a suitable length of time. The biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

Thus, according to a particular embodiment, the plurality of nanoparticles are incubated in the test biofluid sample ex vivo/in vitro under conditions to allow a biomolecule corona to form on the surface of said nanoparticles.

In accordance with the first aspect of the invention, the corona may be digested prior to step (c) in order to facilitate analysis.

In one embodiment, the subject is suffering from a disease and optionally, after step (c) the abundance of the one or more biomarkers is compared to the abundance of the one or more biomarkers in a non-diseased control reference.

In embodiments where the non-diseased control reference comprises a biomolecule corona obtained from a healthy subject, said corona may be digested prior to the equivalent steps of its own analysis.

In some embodiments, albumin and/or immunoglobins may not be depleted from corona samples (which may include for example a corona from a healthy subject) prior to analysis.

The methods of the first aspect of the invention may also be useful for monitoring changes in the amount of the biomarkers, for example in response to therapy. Therefore, in some embodiments, the method may comprise an extra step, during or (preferably before step (a) of administering a therapy to the subject, for example administering a drug molecule, such as for example, an anti-cancer compound. Suitable anti-cancer compounds include, but are not limited to, compounds with activity in cancers such as lung cancer, melanoma or ovarian cancer. In some embodiments, the anti-cancer compound is doxorubicin.

The results obtained in step (c) can be compared to a non-diseased control reference which may comprise the results of corona analysis obtained from a healthy subject. The corona obtained from a healthy subject may be obtained by the same or similar method steps as steps (a) and (b) of the method and may be analyzed by the same or similar method step as step (c) of the method. The healthy subject may be a subject who does not have the type of disease (e.g. cancer) for which the likelihood thereof is being assessed, who does not have any form of disease and/or who does not have any serious illnesses or diseases (e.g. a subject who is generally considered, for example by doctors or other medical practitioners, to be healthy and/or substantially free from disease or illness or serious disease or illness).

A further step (d) may comprise determination and/or calculation of relative or differential abundance between the corona and the non-diseased control reference (such as analysis results of a corona obtained by the same or similar method steps as steps (a) to (c) of the method, but wherein the subject is a healthy subject from a healthy subject) with respect to the or each of the one or more biomarkers. Step (c) and/or (d) may comprise the use of a computer program or software tool. Step (c) and/or (d) may comprise analysis (such as computer or software analysis) of raw data obtained from analyses and/or measurements, for example raw data obtained from LC/MS of the or each corona. Step (c) and/or (d) may comprise a statistical comparison between the protein abundance of the one or more protein biomarkers in the corona and in the non-diseased control reference.

The corona may be digested prior to step (c) and/or step (d), in order to facilitate analysis. In embodiments where the non-diseased control reference comprises a protein corona obtained from a healthy subject, said corona may be digested prior to the equivalent steps of its own analysis.

In particular embodiments of any aspect of the invention, the biomolecule corona is subjected to proteomic analysis, such as via LC-MS/MS or a bicinchoninic acid assay (BCA assay), such as further described herein.

In particular embodiments of any aspect of the invention, the biomolecule corona is subjected to lipidic analysis, such as via UPLC/ESI-MS/MS

In particular embodiments of any aspect of the invention, the biomolecule corona is subjected to genomic analysis, such as via LC-MS/MS or sequence analysis, such as further described herein. Stroun et al. (Neoplastic characteristics of the DNA found in the plasma of cancer patients. Oncology. 46 (5): 318-322, 1989) described that certain characteristics of tumour DNA could be found in a patient's cfDNA. Subsequent publications have confirmed that tumour cells can release their DNA into the circulation. In 1996 Chen et al. (Nat. Med 2:1033-1035, 1996) and Nawroz et al. (Nat. Med 2:1035-1037, 1996) reported the presence of tumour-associated microsatellite alterations, such as loss of heterozygosity (LOH) and microsatellite shifts, in serum and plasma of cancer patients. Circulating free DNA is therefore a useful source material for cancer diagnosis and monitoring.

The inventors have found that analysis of the liposome corona formed in plasma samples obtained from ovarian carcinoma patients revealed higher total cfDNA content compared to healthy controls, suggesting a disease-specific biomolecule corona.

Thus, according to particular embodiments, the method can be used to diagnose or monitor a disease, such as cancer. Suitable cancers include ovarian, lung, prostate, melanoma and blood cancer, including leukemia, lymphoma and myeloma.

The method may be useful in the early detection of a diseased state such as the presence of a tumour in a human subject or for monitoring disease progression and/or response to treatment without the need for invasive tissue sampling, e.g. a biopsy.

According to a second aspect of the invention there is provided a method for detecting a disease state in a subject, comprising:

(a) contacting a biofluid sample from the subject with a plurality of nanoparticles under conditions to allow a biomolecule corona to form on the surface of said nanoparticles; and

(b) analyzing the biomolecule corona for one or more disease-specific biomarkers from two or more biomolecule classes, which is determinative of the presence of a disease in said subject.

In one embodiment, there is provided a method for detecting a disease state in a subject, comprising:

(a) contacting a biofluid sample from the subject with a plurality of nanoparticles under conditions to allow a biomolecule corona to form on the surface of said nanoparticles; and

(b) analyzing the biomolecule corona for one or more disease-specific biomarkers from two or more biomolecule classes, which is determinative of the presence of a disease in said subject wherein the biomolecule corona is analyzed by two or more of proteomic, genomic and lipidomic analysis.

In a particular embodiment, the disease state is cancer. In particular embodiments, the cancer is selected from the group consisting of: lung, ovarian, prostate, melanoma and blood cancer, including leukemia, lymphoma and myeloma.

The method can be used to monitor disease progression, for example to monitor the efficacy of a therapeutic intervention. Suitably the disease is cancer. In a particular embodiment, the cancer is ovarian cancer. Suitably the method involved detecting one or more tumour-specific biomarker over time.

Optionally, after step (a) and before step (b) the nanoparticles and surface-bound biomolecule corona are isolated.

Any isolation technique that is capable of preserving the surface-bound biomolecule corona is suitable. Conveniently, the nanoparticles with surface-bound biomolecule corona are isolated from the biofluid and purified to remove unbound and highly abundant biomolecules (for example albumin) to allow identification of lower abundant biomarkers. The method therefore allows minimization of any masking caused by the highly abundant proteins. Conveniently, the isolation is achieved by a method comprising size exclusion chromatography followed by ultrafiltration.

As with the first aspect of the invention, step (a) of this second aspect of the invention involve administering a plurality of nanoparticles to a subject to allow a biomolecule corona to form on the surface of said nanoparticles or incubating a plurality of nanoparticles in a biofluid sample taken from a subject to allow a biomolecule corona to form on the surface of said nanoparticles. Suitable routes of administration include but are not limited to intravenous, oral, intracerebral (including spinal), intraperitoneal and intra-occular. Conveniently, the route of administration is by intravenous injection. The biomolecule corona typically forms within less than 10 minutes from administration.

In a further embodiment of the second aspect, step (a) comprises incubating a plurality of nanoparticles in a biofluid sample taken from a subject to allow a biomolecule corona to form on the surface of said nanoparticles. Suitably, such incubation can be carried out ex vivo or in vitro (herein the term in vitro includes ex vivo). In this approach, the NP corona is formed in vitro by incubating the plurality of nanoparticles in a biofluid sample to be analyzed. Conveniently, this involves incubating at a suitable temperature, such as at about 37° C., for a suitable length of time. The biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

In one embodiment of any aspect of the invention, when the corona is subjected to nucleic acid analysis (e.g. genomics), the NA level is determined based on quantifying at least one cancer-associated mutation. Suitably, the quantification of the NA level is done at different time points so as to monitor disease progression. In one embodiment of any aspect of the invention, the nucleic acid being detected in cell-free nucleic acid, such as cfDNA or cfRNA. In another embodiment of any aspect of the invention, when the corona is subjected to protein analysis (e.g. proteomics), a protein, polypeptide or protein possessing, or indicative of a disease-associated mutation is detected. In another embodiment of any aspect of the invention, the biomolecule corona is analyzed at the nucleic acid and protein level. In another embodiment of any aspect of the invention, the biomolecule corona is analyzed at the nucleic acid and lipid level. In another embodiment of any aspect of the invention, the biomolecule corona is analyzed at the protein and lipid level. In another embodiment of any aspect of in the invention, the biomolecule corona is analyzed at the protein, lipid and nucleic acid level.

According to a third aspect of the invention there is provided a method for monitoring disease progression in a subject, comprising:

(a) contacting a biofluid sample from the subject with a plurality of nanoparticles under conditions to allow a biomolecule corona to form on the surface of said nanoparticles; and

(b) analyzing the biomolecule corona for one or more disease-specific biomarkers from two or more biomolecule classes; wherein the degree of cancer progression is determined based on the level of the disease-specific biomarker(s) relative to a reference amount.

In one embodiment, there is provided a method for monitoring disease progression in a subject, comprising:

(a) contacting a biofluid sample from the subject with a plurality of nanoparticles under conditions to allow a biomolecule corona to form on the surface of said nanoparticles; and

(b) analyzing the biomolecule corona for one or more disease-specific biomarkers from two or more biomolecule classes;

wherein the degree of cancer progression is determined based on the level of the disease-specific biomarker(s) relative to a reference amount

wherein the biomolecule corona is analyzed by two or more of proteomic, genomic and lipidomic analysis.

As with the first and second aspects of the invention, step (a) of this third aspect of the invention may involve administering a plurality of nanoparticles to a subject to allow a biomolecule corona to form on the surface of said nanoparticles or incubating a plurality of nanoparticles in a biofluid sample taken from a subject to allow a biomolecule corona to form on the surface of said nanoparticles. Suitable routes of administration include but are not limited to intravenous, oral, intracerebral (including spinal), intraperitoneal and intra-occular. Conveniently, the route of administration is by intravenous injection. The biomolecule corona typically forms within less than 10 minutes from administration.

In an alternative embodiment, step (a) comprises incubating a plurality of nanoparticles in a biofluid sample taken from a subject to allow a biomolecule corona to form on the surface of said nanoparticles. Suitably, such incubation can be carried out ex vivo or in vitro (herein the term in vitro includes ex vivo). In this approach, the NP corona is formed in vitro by incubating the plurality of nanoparticles in a biofluid sample to be analyzed. Conveniently, this involves incubating at a suitable temperature, such as at about 37° C., for a suitable length of time. The biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

Optionally, after step (a) and before step (b) the nanoparticles and surface-bound biomolecule corona are isolated.

In a particular embodiment, the disease is cancer. In particular embodiments, the cancer is selected from the group consisting of: lung, ovarian, prostate, melanoma and blood cancer, including leukemia, lymphoma and myeloma.

In a particular embodiment, the reference amount is the amount detected at a previous time point, for example, at least 1 week, 2 weeks, 1 month, 3 months, 6 months, 12 months, 18 months, or 24 months earlier.

In a particular embodiment, if the total amount of the biomarker being measured (analyzed) has increased compared to the reference amount it signifies that the patient's disease has progressed and if the total amount of the biomarker has decreased compared to the reference amount the patient's disease has regressed.

Any isolation technique that is capable of preserving the surface-bound biomolecule corona is suitable. Conveniently, the nanoparticles with surface-bound biomolecule corona are isolated from the biofluid and purified to remove unbound and highly abundant biomolecules (for example albumin) to allow identification of lower abundant biomarkers. The method therefore allows minimization of any masking caused by the highly abundant proteins. Conveniently, the isolation is achieved by a method comprising size exclusion chromatography followed by ultrafiltration.

In a particular embodiment of any of the aspect of the invention, the nanoparticles with surface-bound biomolecule corona are isolated from the biofluid and purified to remove unbound and highly abundant biomolecules to allow identification of low abundant biomarkers.

In a particular embodiment of any of the aspect of the invention, the nanoparticles with surface-bound biomolecule corona are isolated from the biofluid and purified by a method comprising size exclusion chromatography followed by ultrafiltration.

The method of the second and third aspects of the invention may offer high sensitivity and a high level of precision which allows for the identification, detection and/or quantification of the disease markers, e.g. cancer biomarkers and/or the abundance thereof, even when present in low abundance, which otherwise may be very difficult to identify.

In particular embodiments, the disease is cancer selected from the group consisting of: lung, ovarian, prostate, melanoma and blood cancer, including leukemia, lymphoma and myeloma.

In a particular embodiment of any aspect of the invention, the method may further comprise a step of determining the abundance (such as normalised abundance, mean normalised abundance, % abundance, for example) of the or each analyzed biomarker in the corona.

When the biofluid sample is from a subject with or suspected of having a disease the abundance of one or more biomarkers in the corona can be compared to the abundance of the same one or more biomarkers in a non-diseased control reference.

In particular embodiments of any aspect of the invention, at least one of the biomarker(s) is a complex between nucleic acid and a protein or protein derivative.

In particular embodiments of any aspect of the invention, the method may comprise determining the abundance of at least 1, 2, 3, 5, 10, 20, 30, 40, 50, 75, 100, 150, 200, 250, 300 or at least 350 biomarkers, and optionally, comparing the results with the abundance of the same biomarkers in a non-diseased control reference.

In a particular embodiment of any aspect of the present invention, the analysis is conducted on a single biofluid sample. Suitably, the sample is a plasma sample.

In a particular embodiment, the invention relates to a method of identifying a new biomarker from a biofluid, wherein the method comprises:
(a) isolating a plurality of nanoparticles with surface-bound biomolecule corona from a biofluid sample taken from a subject in a diseased state; and
(b) analyzing the biomolecule corona to identify biomarkers from two or more distinct biomarker classes;
(c) identifying one or more new biomarkers.

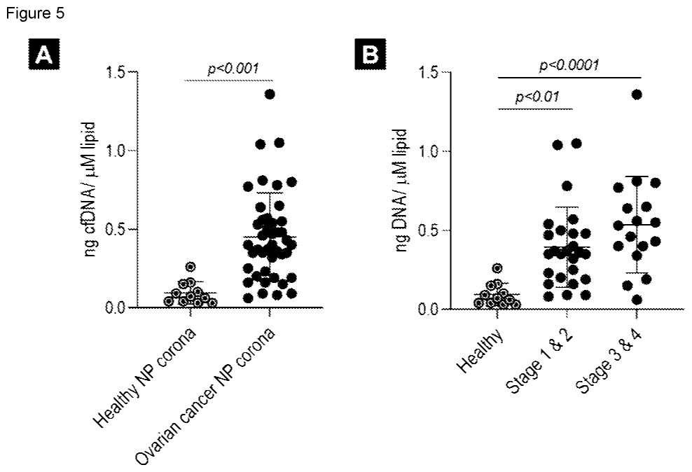

Surprisingly, inventors have found that the total cfDNA biomolecule content of the biomolecule corona isolated after administering a plurality of nanoparticles to ovarian cancer subjects to allow a biomolecule corona to form on the surface of the nanoparticles is significantly higher in comparison to healthy subjects. FIG. 5 shows data to illustrate this surprising discovery. When normalised to post-purification liposome concentration, cfDNA was significantly higher in ovarian cancer samples (all stages, early stage (I and II) and late-stage (III and IV)) compared to healthy controls (p values=<0.001, <0.01 and <0.0001, respectively). Similar findings were found with total protein levels.

The protein and/or cfNA content adsorbed onto the nanoparticle can therefore be used to detect or diagnose the disease state. Protein and/or cfNA detection in the NP corona can therefore be used to indicate the presence of disease in a subject.

Proteomic Analysis

The various aspects of the invention are directed to the detection/identification of one or more biomarkers. In a particular embodiment of any aspect of the invention, at least one of the biomarker(s) is a protein or protein derivative.

In a particular embodiment of any aspect of the present invention, at least one of the biomolecule classes analyzed is protein and the protein or protein derivative is analyzed directly without prior extraction or purification from the NP corona.

Analysis of the biomolecule corona in order to identify proteinaceous biomarkers can be carried out using any suitable technique capable of detecting said biomarkers.

The total protein biomolecule content of the biomolecule corona can be determined by any method capable of quantifying the level of said biomolecules in the surface-bound corona. In one embodiment, the total protein content is determined by bicinchoninic acid (BCA) assay. In one particular embodiment, the subject is a human patient and the total protein content is at least 700, 800, 900, 1000, 1250, 1500, 1800, 2000, 25000 or 3000 Pb when measured using a BCA assay.

In addition to a determination of the total biomolecule content of the biomolecule corona, analysis of the biomolecule corona can also reveal qualitative and quantitative information regarding specific potential biomarkers. Such analysis can be carried out using any suitable techniques of capable of detecting said biomarkers. Protein mass spectrometry is often used for the accurate mass determination and characterization of molecules, including proteins, and a variety of methods and instrumentations have been developed for its many uses.

In a particular embodiment of the invention, the biomolecule corona is analyzed by gel electrophoresis, mass spectrometry, an immunoassay, UV-Vis. absorption, fluorescence spectroscopy, chromatography or NMR methodology. Conveniently, the biomolecule corona is analysed by mass spectrometry, which can allow qualitative and quantitative analysis of the biomolecule corona present on the nanoparticles. In a particular embodiment, the methods allow identification of unique biomolecules without the need for highly specialized and ultra-sensitive analytical mass spectrometry instrumentation such as using an UltiMate® 3000 Rapid Separation LC (RSLC, Dionex Corporation, Sunnyvale, Calif.) coupled to a LTQ Velos Pro (Thermo Fisher Scientific, Waltham, Mass.) mass spectrometer.

In one aspect of this embodiment, analysis of the biomolecule corona is carried out after administering a plurality of nanoparticles to a subject in a diseased state to allow a biomolecule corona to form on the surface of said nanoparticles and isolating the nanoparticles and surface-bound biomolecule corona. When compared to other methods, such methods can yield high levels of unique low abundant biomolecules and allow identification of such unique biomolecules without the need for highly specialized and ultra-sensitive analytical mass spectrometry instrumentation such as an UltiMate® 3000 Rapid Separation LC (RSLC, Dionex Corporation, Sunnyvale, Calif.) coupled to a LTQ Velos Pro (Thermo Fisher Scientific, Waltham, Mass.) mass spectrometer.

In a particular embodiment of the invention, the beneficial sensitivity and high level of precision provided by the method allows the identification of intracellular protein disease related biomarkers that are present in low abundance and would otherwise be very difficult to identify. Conveniently, the method allows identification of protein biomarkers with molecular weight of less than 80 kDa. More conveniently, the method allows identification of protein biomarkers with molecular weight of less than 40 kDa or less than 20 kDa.

Surprisingly, inventors have also found that the total protein content determined by administering a plurality of nanoparticles to a subject is greater than if determined by incubating the plurality of nanoparticles in-vitro with a biofluid taken from the subject. In a particular embodiment, the total protein content determined is at least between 1.2 and 5 fold higher than if determined by incubating the plurality of nanoparticles in-vitro with a biofluid isolated from the subject. Conveniently, total protein content determined is at least 1.5, 1.8, 2, 3, 4 or 5 fold higher than if determined by incubating the plurality of nanoparticles in-vitro with a biofluid isolated from the subject. Conveniently, the subject in this embodiment is a human.

Genomic/Nucleic Acid Analysis

The various aspects of the invention are directed to the detection/identification of one or more biomarkers. In a particular embodiment of any aspect of the invention, at least one of the biomarker(s) is nucleic acid. Suitably, the biomarker is a nucleic acid target region. In a particular embodiment of any aspect of the invention, at least one of the biomarker(s) is cell-free nucleic acid (cfNA). Suitably, in any of the aspects of the invention, the cfNA is cell free ribonucleic acid (cfRNA) or cell free deoxyribonucleic acid (cfDNA). cfRNA can be any cell-free RNA including microRNA. cfDNA can be any cell free DNA, including genomic DNA. Suitably, the cfNA is fragmented. In a particular embodiment, the cfNA is nucleic acid released from a cancer cell. Such nucleic acid may comprise or house one or more mutations associated with the cancer.

The nucleic acid (such as cell free nucleic acid) that forms or adsorbs onto the nanoparticles (either directly or indirectly by association with another biomolecules, such as a protein) can be subjected to genetic analysis by any technique of interest. Such analysis could be quantitating total nucleic acid, sequencing of the nucleic acid and/or undertaking one or more targeted genetic analyzes using known molecular diagnostic techniques to test the genetic state of an individual, including assessing for genetic diseases; mendelian disorders; genetic mosaicism; predicting response to drug treatment; and/or diagnosing or monitoring a medical condition. In addition, the nucleic acid-based cancer diagnostics contemplated herein possess the ability to detect a variety of genetic changes including somatic sequence variations that alter protein function, large-scale chromosomal rearrangements that create chimeric gene fusions, and copy number variation that includes loss or gain of gene copies.

When analysing nucleic acid, it may be preferably to fragment the target nucleic acid. Nucleic acids, including genomic nucleic acids, can be fragmented using any of a variety of methods, such as mechanical fragmenting, chemical fragmenting, and enzymatic fragmenting. Methods of nucleic acid fragmentation are known in the art and include, but are not limited to, DNase digestion, sonication, mechanical shearing, and the like.

Genomic nucleic acids can be fragmented into uniform fragments or randomly fragmented. In certain aspects, nucleic acids are fragmented to form fragments having a fragment length and/or ranges of fragment lengths as required depending on the type of nucleic acid targets one seeks to capture and the design and type of probes such as molecular inversion probes (MIPs) that will be used. Chemical fragmentation of genomic nucleic acids can be achieved using methods such as a hydrolysis reaction or by altering temperature or pH. Nucleic acid may be fragmented by heating a nucleic acid immersed in a buffer system at a certain temperature for a certain period to time to initiate hydrolysis and thus fragment the nucleic acid. The pH of the buffer system, duration of heating, and temperature can be varied to achieve a desired fragmentation of the nucleic acid. Mechanical shearing of nucleic acids into fragments can be used e.g., by hydro-shearing, trituration through a needle, and sonication. Nucleic acid may also be fragmented enzymatically. Enzymatic fragmenting, also known as enzymatic cleavage, cuts nucleic acids into fragments using enzymes, such as endonucleases, exonucleases, ribozymes, and DNAzymes. Varying enzymatic fragmenting techniques are well-known in the art.

In certain embodiments, the sample nucleic acid is captured or targeted using any suitable capture method or assay such as amplification with PCR, hybridization capture, or capture by probes such as MIPs.

In a particular embodiment of any aspect of the present invention, the nucleic acid in the NP corona is isolated and fragmented before analysis.

In a particular embodiment of any aspect of the invention, the nucleic acid content of the biomolecule corona is quantitated using qPCR, such as real time qPCR. In one embodiment, the nucleic acid is cfNA, such as cfDNA.

Prior to the analysis of the nucleic acid in the surface-bound biomolecule corona it may be desirable to amplify the nucleic acid using the well-established technique of polymerase chain reaction (PCR). Alternatively, a nucleic acid library of the nucleic acid in the surface-bound biomolecule corona could be generated.

A suitable DNA library could be generated by the end-repair of isolated DNA, wherein fragmented DNA (e.g. cfDNA) is processed by end-repair enzymes to generate end-repaired DNA with blunt ends, 5′-overhangs, or 3′-overhangs which can then be cloned into a suitable vector, e.g. plasmid, and used to generate a DNA clone library. Optionally, an adaptor is ligated to each end of an end-repaired DNA, and each adaptor comprises one or more PCR or sequencing primer binding sites. If desired, PCR can then amplify the initial DNA library. The amount of amplified product can be measured using methods known in the art, e.g., quantification on a Qubit 2.0 or Nanodrop instrument.

In particular embodiments, a method for genetic analysis of DNA comprises: generating and amplifying a DNA library, determining the number of genome equivalents in the DNA library; and performing a quantitative genetic analysis of one or more target loci.

In particular embodiments, a method for genetic analysis of DNA comprises treating DNA with one or more end-repair enzymes to generate end-repaired DNA and ligating one or more adaptors to each end of the end-repaired DNA to generate a DNA library; amplifying the DNA library to generate DNA library clones; determining the number of genome equivalents of DNA library clones; and performing a quantitative genetic analysis of one or more target genetic loci in the DNA library clones.

The nucleic acid captured in the corona can be subjected to nucleotide sequencing by any method known in the art. DNA sequencing techniques include classic dideoxy sequencing reactions (Sanger method) using labelled terminators or primers and gel separation in slab or capillary, sequencing by synthesis using reversibly terminated labelled nucleotides or using allele specific hybridization to a library of labelled clones, Illumina/Solexa sequencing, pyrosequencing, 454 sequencing, and SOLiD sequencing. Separated molecules may be sequenced by sequential or single extension reactions using polymerases or ligases as well as by single or sequential differential hybridizations with libraries of probes.

An example of a suitable sequencing technique is Illumina sequencing which is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labelled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated. Sequencing according to this technology is described in various patent publications including: U.S. Pat. Nos. 7,960,120; 7,835,871; 7,232,656 and 6,210,891.

With the advances in next generation sequencing, the cost of sequencing whole genomes has decreased dramatically, however the cost and time involved in sequencing entire genomes may not be practical or necessary. Instead, different genome partitioning techniques can be used to isolate smaller but highly specific regions of the genome for further analysis. Molecular Inversion Probe (MIP) technology, for instance, can be used to capture a small region of the genome for further examination, such as single nucleotide polymorphism (SNP) genotyping, allelic imbalance studies or copy number variation assessments (e.g. Hardenbol et al., “Highly multiplexed molecular inversion probe genotyping: over 10,000 targeted SNPs genotyped in a single tube assay”. Genome Res 15:269-75, 2005).

In a particular embodiment of any aspect of the present invention, at least one of the biomolecule classes analyzed is nucleic acid and the amount or relative amount of total cfNA is determined.

In a particular embodiment of any aspect of the present invention, at least one of the biomolecule classes analyzed is nucleic acid and the amount or relative amount of total cfDNA is determined.

In a particular embodiment of any aspect of the present invention, the amount of at least one biomarker in the corona is quantitated directly without prior extraction or purification.

In a particular embodiment of any aspect of the present invention, at least one of the biomolecule classes analyzed is nucleic acid and the nucleic acid is analyzed directly without prior extraction or purification from the NP corona.

In a particular embodiment of any aspect of the present invention, at least one of the biomolecule classes analyzed is cfDNA and the cfDNA is analyzed directly without prior extraction or purification from the NP corona.

In a particular embodiment of any aspect of the present invention, a specific nucleic acid sequence within the biofluid is detected. Suitably, the specific nucleic acid is indicative of a disease, such as being or comprising a disease-associated mutation. One example is the detection of activating mutations in epidermal growth factor receptor (EGFR) gene in certain patients with non-small cell lung cancer (NSCLC). Key activating mutations in EGFR include: a deletion in exon 19 (e.g. Del (746-750)) and the L858R point mutation that constitute approximately 90% of all EGFR activating mutations in NSCLC patients. The methods of the invention can be used to detect one or more EGFR activating mutations, or indeed, resistance mutations, and so can be used for diagnosis or monitoring purposes.

The present invention includes methods for identifying a cell free nucleic acid biomarker in a biofluid.

In a particular embodiment of any aspect of the invention the cfNA is adsorbed onto the surface of a nanoparticle. Suitably, the cfNA is adsorbed onto the nanoparticle surface as part of a Nucleic Acid-protein complex. In particular embodiments, the Nucleic Acid-protein complex comprises one or more histone proteins, such as H2, H2B, H4, histone-lysine N-methyltransferase 2D and histone PARylation factor 1. In particular embodiments, the Nucleic Acid-protein complex is a DNA-protein complex.

The total biomolecule content of the cfNA biomolecule corona can be determined by any method capable of quantifying the level of said biomolecules in the surface-bound corona. In one embodiment, the biomolecule method involves determining the total nucleic acid content and this is suitably determined by qPCR. Total NA content can be gauged by measuring a reference gene, such as the RNase P gene (e.g. using The Applied Biosystems® TaqMan™ RNase P Detection Reagents Kit).

In one embodiment, the cfNA is detected directly from the NP corona. In another embodiment, the cfNA is purified from the corona before analysis. Purification of nucleic acid is well-known. A suitable kit for purifying circulating nucleic acid in a sample is QIAamp circulating nucleic acid extraction kit (QIAGEN).

Unique cfNA biomarkers can also be detected by nucleic acid sequencing, either direct on the corona or following polymerase chain reaction amplification of cfNA in the corona.

In a particular embodiment of the invention, the beneficial sensitivity and high level of precision provided by the method allows the identification of intracellular cfNA disease related biomarkers that are present in low abundance and would otherwise be very difficult to identify.

Lipid Analysis.

The various aspects of the invention are directed to the detection/identification of one or more biomarkers. In a particular embodiment of any aspect of the invention, at least one of the biomarker(s) is a lipid.

Lipids are typically analysed by chromatographic methods. The most common chromatographic methods for lipid analysis are thin-layer chromatography (TLC), GC, and high-performance liquid chromatography (HPLC), used alone or in conjugation with mass spectrometry (MS), tandem quadrupoles (MS/MS), flame ionization detector (FID), and time-of-flight (TOF). In a particular embodiment, the analysis is ultra-performance liquid chromatography-electrospray ionization-tandem mass spectrometry (UPLC-ESI-MS/MS).

In a particular embodiment of any aspect of the present invention, at least one of the biomolecule classes analyzed is lipid and the lipid is analyzed directly without prior extraction or purification from the NP corona.

Metabolomics

Metabolomic analyses typically utilize nuclear magnetic resonance (NMR)-based detection, or gas or liquid chromatography coupled to mass spectrometry (MS), e.g. LC-MS and LC-MS/MS, which typically allows the detection of 3000-5000 molecules per experiment. MS-based approaches outperform NMR in terms of sensitivity and can be run in an untargeted or targeted approach. A commercial or in-house targeted approach set up might interrogate between 10 and several hundred metabolites per run.

Biofluid

The biofluid can be any fluid obtained or obtainable from a subject. The subject can be an animal. In a particular embodiment of any aspect of the invention the subject is a human. In particular embodiments, the subject is suffering from a disease (in a diseased state).

In particular embodiments of any aspect of the invention, the biofluid is selected from blood, plasma, serum, saliva, sputum, urine, ascites, lacrimal, cerebrospinal and ocular fluids. In a particular embodiment, the biofluid is plasma.

Suitably the biofluid is a blood or blood fraction sample, such as serum or plasma.

In a particular embodiment, the biofluid has been produced from a solid tissue, such as a solid tumor tissue, by treatment to macerate/lyse the tissue to generate a fluid.

Nanoparticles

A plurality of nanoparticles can be a population of the same type of nanoparticle (a population of nanoparticles) or more than one population of nanoparticles, wherein each population is of a different type of nanoparticle; and so when combined can be termed a heterogeneous population of nanoparticles (i.e. a plurality of distinct nanoparticle populations).

Certain classes of nanoparticle are more effective at adsorbing different biomolecules, therefore by utilizing a mixture of distinct nanoparticles (i.e. two or more distinct nanoparticle populations) it will be possible to create a corona that comprises a particular complement of biomolecules and/or as many biomolecule species as possible.

Thus, in a particular embodiment the plurality of nanoparticles used is a heterogeneous population of nanoparticles.

In a particular embodiment, all the nanoparticles used in the method are of the same type of nanoparticle, and so can be termed a homogeneous population of nanoparticles.

In one embodiment, there is provided a method of identifying biomarkers from two or more distinct biomolecule classes in a biofluid, wherein the method comprises:
(a) contacting a plurality of nanoparticles with a biofluid to allow a biomolecule corona to form on the surface of said nanoparticles;
(b) isolating the nanoparticles and surface-bound biomolecule corona; and
(c) analyzing the biomolecule corona to identify biomarkers from two or more distinct biomarker classes;

wherein the plurality of nanoparticles is a homogeneous population of nanoparticles.

In one embodiment, there is provided a method for detecting a disease state in a subject, comprising:
(a) contacting a biofluid sample from the subject with a plurality of nanoparticles under conditions to allow a biomolecule corona to form on the surface of said nanoparticles; and
(b) analyzing the biomolecule corona for one or more disease-specific biomarkers from two or more biomolecule classes, which is determinative of the presence of a disease in said subject;

wherein the plurality of nanoparticles is a homogeneous population of nanoparticles.

In one embodiment, there is provided a method for monitoring cancer progression in a subject, comprising:
(a) contacting a biofluid sample from the subject with a plurality of nanoparticles under conditions to allow a biomolecule corona to form on the surface of said nanoparticles; and
(b) analyzing the biomolecule corona for one or more cancer-specific biomarkers from two or more biomolecule classes;

wherein the degree of cancer progression is determined based on the level of the cancer-specific biomarker(s) relative to a reference amount; and

wherein the plurality of nanoparticles is a homogeneous population of nanoparticles.

The methods are applicable to any types of nanoparticles capable of attracting a biomolecule corona. In a particular embodiment of any aspect of the invention, the nanoparticles are selected from liposomes, metallic nanoparticles (such as gold or silver nanoparticles), polymeric nanoparticles, fibre shaped nanoparticles (such as carbon nanotubes) and 2-dimensional nanoparticles (such as graphene oxide nanoparticles) or any combination thereof. In a particular embodiment, the nanoparticles are PEGylated liposomes.

Suitably, the nanoparticles comprise liposomes. Conveniently, the nanoparticles are liposomes. Liposomes are generally spherical vesicles comprising at least one lipid bilayer. Liposomes are often composed of phospholipids. In a particular embodiment, the liposomes are composed of phospholipid molecules and functionalised amphiphilic molecules (eg. PEGylated DSPE). In a particular embodiment, the liposomes are composed of phospholipid molecules and functionalised amphiphilic molecules (eg. PEGylated DSPE) that are able to self-assemble into unilamellar vesicles. In a particular embodiment, the liposomes are PEGylated DSPE. Conveniently, the liposomes are able to encapsulate drug molecules in their inner aqueous phase, and in some embodiments may contain one or more drug molecules therein. In one embodiment, the drug molecule is doxorubicin, or a pharmaceutically acceptable salt thereof. In one embodiment, the drug molecule is doxorubicin hydrochloride.

The inventors have found that NA-containing coronas form on negatively charged nanoparticles. As nucleic acid is negatively charged this is surprising. In a particular embodiment, the nanoparticles are negatively charged.

Biomolecule Corona

The corona formed on the nanoparticles is a biomolecule corona. Conveniently, the biomolecule corona will typically comprise different classes of biomolecule, such as proteins, peptides, fatty acids, lipids, amino acids, amides, sugars and nucleic acids. Conveniently the biomolecule corona comprises proteins and/or lipids and/or nucleic acid, such as cell free nucleic acid (e.g. cfDNA and/or cfRNA). Conveniently the biomolecule corona comprises one or more measurable biomarkers.

As mentioned elsewhere herein, the biomolecule corona can form almost immediately, but typically the incubation is carried out for a period of 5-60 minutes, or more; such as 5, 10, 15, 20, 30, 40, 50, 60 or more minutes. Conveniently, the mixture can be subject to agitation, for example by way of an orbital shaker set at approximately 250 rpm to mimic in vivo conditions. Suitably, the biofluid sample from the subject to be analyzed has been previously taken and the sample extraction step is not part of the method.

In the methods of the invention that involve administration of the nanoparticles to a subject, a biofluid sample comprising some of the introduced nanoparticles is then extracted from the subject; for example, by taking a blood sample, after a period of time to allow the corona to form. In particular embodiments, the biofluid sample comprising nanoparticles is extracted/removed from the subject at least 5 minutes after administration, such as at least 5, 6, 7, 8, 9, 10, 12, 15, 20, 30, 40, 60, 90, 120 minutes or more, after the nanoparticles were administered to the subject. The volume of the biofluid sample comprising nanoparticles extracted can be determined by the physician and will depend on the source of the biofluid sample. For example, if it is a blood sample, it may be in a volume of 2-20 ml. In a particular embodiment, the nanoparticles are isolated from the biofluid sample prior to analysis.

In particular embodiments, the methods of the invention comprise administering a plurality of nanoparticles to a subject, a biofluid sample is then taken from the subject and analysed. Prior to analysis, the particles are isolated from the biofluid and purified to remove unbound and highly abundant biomolecules. In one embodiment the plurality of nanoparticles are administered to the subject by intravenous injection.

Multi-Omic Analysis

Once the biomolecule corona has been formed the sample can be split into portions and each portion subjected to a particular-omic analysis as describe herein. In certain circumstances, it may be possible to simultaneously analyze one sample by more than one-omic analysis. Thus, the analysis from two or more distinct biomarker classes can be done on the same sample containing the nanoparticle-biomolecule corona, or it can be carried out separately on distinct portions of the original sample.

A biomarker, or biological marker, generally refers to a qualitative and/or quantitative measurable indicator of some biological state or condition. Biomarkers are typically molecules, biological species or biological events that can be used for the detection, diagnosis, prognosis and prediction of therapeutic response of diseases. Most biomarker research has been focused on measuring a concentration change in a known/suspected biomarker in a biological sample associated with a disease. Such biomarkers can exist at extremely low concentrations, for example in early stage cancer, and accurate determination of such low concentration biomarkers has remained a significant challenge.

In a particular embodiment of any aspect of the present invention, the relative amount of a biomarker in the sample is determined by reference to a control amount in the sample. A control nucleic acid may be a nucleic acid sequence, such as a gene, that is representative of a wild-type/healthy level. A control protein may be a protein that is representative of a wild-type/healthy level. A control lipid may be a lipid that is representative of a wild-type/healthy level.

In particular embodiments of any aspect of the invention, the method may comprise determining the abundance of at least 1, 2, 3, 4, 5, 6, 7, 8, 9, 10, 15, 20, 25, 30, 35, 40, 45, 50, 75, 100, 150, 200 or at least 250 biomarkers, and optionally, comparing the results with the abundance of the same biomarkers in a non-diseased control reference.

Monitoring effects of therapy The methods of the invention can be used to monitor the effects of a therapeutic treatment. For example, a determination of one or more biomarkers in a patient's biofluid can be conducted prior to a therapeutic intervention (such as chemotherapy, radiotherapy or administration of any therapeutic drug) and then at one or more time points during or after treatment. A change in the amount of the biomarker(s) detected can then be used to determine the effectiveness of the treatment.

Therefore, in some embodiments, the method may comprise an extra step, during or (preferably before step (a)), of administering a therapy to the subject, for example administering a drug molecule, such as for example, an anti-cancer compound. Suitable anti-cancer compounds include, but are not limited to, compounds with activity in cancers such as lung cancer, melanoma or ovarian cancer. In some embodiments, the anti-cancer compound is doxorubicin.

In a separate embodiment, there is provided a method for monitoring the changes in biomarkers in a subject in response to therapy, comprising the step of (a) contacting a plurality of nanoparticles with a biofluid from a therapeutically treated subject with cancer to allow a biomolecule corona to form on the surface of said nanoparticles.

In a particular embodiment of any aspect of the present invention, at least one of the biomolecule classes analyzed is nucleic acid and a change in total cfNA in a biofluid from a subject in response to therapy is monitored. In a particular embodiment of any aspect of the present invention, a change in cfNA of a cancer-associated genetic marker (e.g. mutation) in a biofluid from a subject in response to therapy is monitored.

In a particular embodiment of any aspect of the present invention, at least one of the biomolecule classes analyzed is protein and a change in total protein content in a biofluid from a subject in response to therapy is monitored.

In a particular embodiment of any aspect of the present invention, at least one of the biomolecule classes analyzed is lipid and a change in total lipid content in a biofluid from a subject in response to therapy is monitored.

In a particular embodiment, the therapy comprises administration of a drug molecule to the subject.

In a particular embodiment, the patient is being treated with an anti-cancer compound. Conveniently, the anti-cancer compound is doxorubicin.

Panels of Biomarkers

In addition to the identification of a single biomarker, the methods of the invention also provide the ability to identify panels of biomarkers (multiplexing). This approach can lead to increased sensitivity and specificity of detection. In a particular embodiment of any aspect of the invention, the biomarker is part of a panel of disease-specific biomolecule biomarkers. In a further embodiment, the panel comprises a combination of unknown and known disease-specific biomolecule biomarkers.

Kits

In a further aspect of the invention, there is provided a diagnostic kit comprising nanoparticles and reagents capable of detecting one or more of the biomolecules listed in Table 2, Table 3, Table 4, Table 5, Table 6, Table 7 or Table 8.

Use of Protein Biomarkers

In a further aspect of the invention, there is provided any one or more of the biomolecules listed in Table 2, Table 3, Table 4, Table 5, Table 6, Table 7 or Table 8, or any combinations thereof, for use as a biomarker.

### EXAMPLES

Materials and Methods

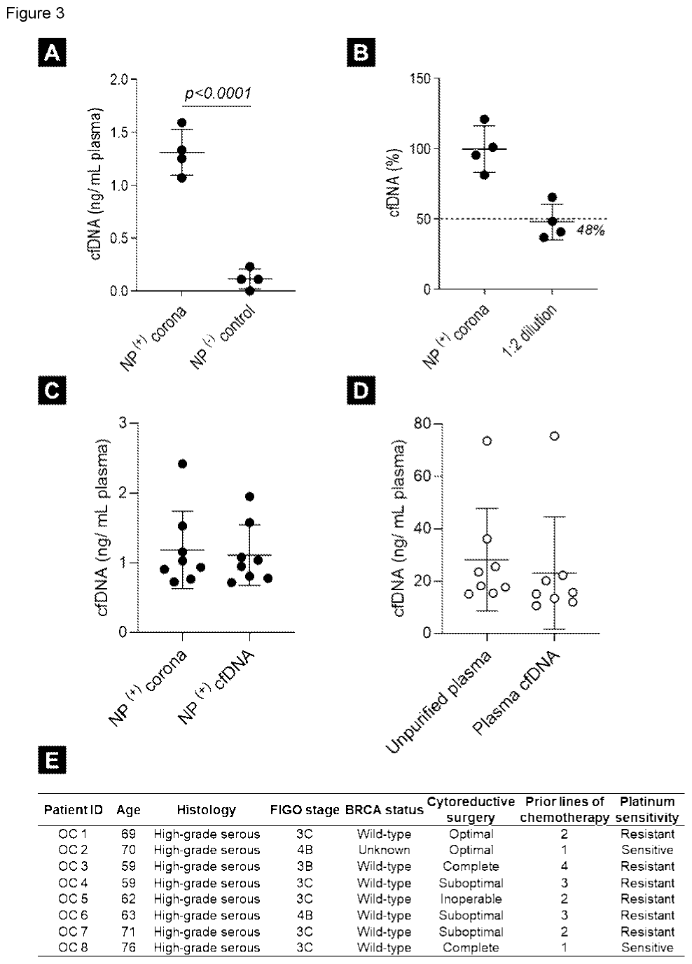

M1. Plasma samples. Healthy human female pooled K2EDTA plasma samples were purchased from BiolVT (West Sussex, UK) (Lot #HMN2528). All ovarian cancer K2EDTA plasma samples were collected by the MCRC Biobank (details provided in Table 1 and FIG. 3E). Individual age- and sex-matched K2EDTA plasma controls (female, 45-85 years old) were purchased from BiolVT (West Sussex, UK) (Table 1). All plasma samples were stored at −80° C.

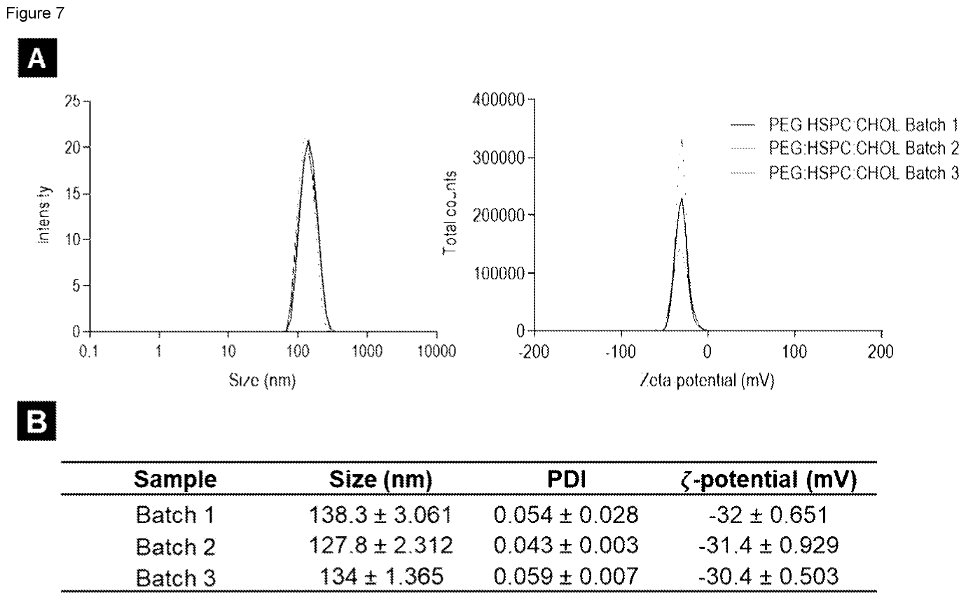

M2. Liposome preparation. HSPC:Chol:DSPE-PEG2000 (56.3:38.2:5.5) liposomes (Doxil® formulation) liposomes were prepared using the thin lipid film method followed by extrusion as described previously.¹⁴ All liposome batches were diluted to 12.5 mM, with the same batch of liposomes used for group comparisons. The physiochemical characteristics of the liposome batches are shown in FIG. 7.

M3. Dynamic light scattering (DLS) for size and zeta-potential measurements. Liposome size and surface charge were measured as described previously.¹⁴ Liposomes were diluted in distilled water and measured in size or capillary cuvettes using the Zetasizer Nano ZS (Malvern, Instruments, UK).

M4. Biomolecule corona formation (liposome plasma incubation and purification). Liposome and plasma incubations and purifications were performed as described previously.¹⁴ In brief, 820 μL human plasma and 180 μL PEGylated liposomes were incubated for 10 mins at 37° C., shaking at 250 rpm. Unbound proteins and other unknown biomolecules were removed by size exclusion chromatography (SEC) (Sepharose CL-4B columns (Sigma-Aldrich)) followed by membrane ultrafiltration (Vivaspin® columns (Sartorious, Fisher Scientific)). Samples were concentrated to 100 μL for characterisation or downstream processing. For characterisation of individual chromatographic fractions, samples were concentrated to 100 μL using 1,000,000 molecular weight cut off (MWCO) Vivaspin® membrane ultrafiltration columns ((Sartorious, Fisher Scientific). Plasma controls were subjected to the same purification process for comparison.

M5. Circulating cell-free nucleic acid extraction. Cell-free nucleic acids were purified from ex vivo plasma samples, liposomal corona samples and plasma control samples using a QIAamp® Circulating Nucleic Acid Extraction kit and QIAvac 24 Plus vacuum manifold according to manufacturer's instructions (QIAGEN, Hilden, Germany). After an initial sample lysis step, cell-free nucleic acids were bound onto a silica-based purification column (QIAGEN mini column). Multiple washing steps were performed prior to elution of cell-free nucleic acids in buffer AVE (QIAGEN). All samples were eluted in a final volume of 50 μL.

M6. Cell-free DNA quantification. Cell-free DNA was measured using two real-time quantitative PCR (qPCR) assays. The single-copy RNase P probe real-time assay was performed using TaqMan® RNase P Detection Reagents kit (Life Technologies) and SensiFAST Probe Hi-ROX master mix (Bioline, Meridian Bioscience). All real-time qPCR reactions included 7.5 μL of 2× SensiFAST probe mastermix, 0.75 μL 20× RNase P primer/probe mix, 1.75 μL nuclease-free water (Ambion, Tex., USA) and 5 μL of sample. Cycling conditions included (95° C., 5 mins)×1, (95° C., s; 60° C., 50 s)×40 and were performed on a LightCycler® 96 (Roche, Basel, Switzerland).

The multi-locus LINE-1 real-time qPCR assay was performed using primers described previously⁷³ purchased from Integrated DNA Technologies (desalted, 25 nmol scale) using a robust Terra qPCR Direct SYBR Premix master mix (Takara Bio, USA). All real-time PCR reactions included 7.5 μL of 2× Terra qPCR Direct SYBR Premix master mix, 0.75 μL of each 10 μM forward and reverse primers), 5.75 μL nuclease-free water (Ambion, Tex., USA) and 1 μL of sample. Cycling conditions included (98° C., 2 mins)×1, (98° C., 10 s; 60° C., 15 s; 68° C., 30 s)×35 and were performed on a LightCycler® 96 (Roche, Basel, Switzerland).

Sample input was either corona-coated liposomes, purified cfDNA or plasma samples diluted 1:40. Plasma samples were only quantified using the LINE-1 real-time PCR assay in combination with the robust Terra qPCR Direct SYBR Premix master mix.

M7. Mass spectrometry. In-gel digestion of corona proteins was performed prior to LC-MS/MS analysis, as described previously.¹⁴ Digested proteins were analyzed by LC-MS/MS using an UltiMate 3000 Rapid Separation LC (RSLC, Dionex Corporation, Sunnyvale, Calif.) plus Q Exactive Hybrid Quadrupole-Orbitrap (Thermo Fisher Scientific, Waltham, Mass., USA) mass spectrometer system. Data were analyzed using Mascot (Matrix Science UK) in combination with the SwissProt_2016_04 database (taxonomy human). Progenisis QI software (version 4.3.2, Proteome Software Inc.) was used for relative protein quantification based on spectral counting and statistical analyzes (One-way analyzes of variance (ANOVA)).

The accession numbers of the proteins indicated in Tables 2-5 were assigned using SwissProt_2016_04 database.

M8. Statistical analysis. Statistical comparisons of these data were performed using GraphPad Prism v.8.2.0. For comparisons of three groups or more, one-way ANOVA tests were performed followed by the Tukey's multiple comparison test (adjusted p values <0.05 were considered significant). For comparisons of two groups unpaired student t-tests were performed (FDR-adjusted p values <0.05 were considered significant). All data averages were presented as mean±standard deviation (SD).

M9. Ethical Approvals: This project has research ethics approval under the Manchester Cancer Research Centre (MCRC) Biobank Research Tissue Bank Ethics (NHS NW Research Ethics Committee 18/NW/0092). All participants provided written informed consent to participate in this study.

### Example 1

1.1 Plasma Incubation and Biomolecule Corona Formation.

To evaluate the cfDNA content of the biomolecule corona, human plasma samples obtained from healthy volunteers were incubated (37° C., 10 minutes, 250 rpm) with PEGylated liposomes (HSPC:Chol:DSPE-PEG2000), a formulation which constitutes the basis of the anti-cancer agent Doxil®. (FIG. 7). Liposomes were employed in this study due to their extensive protein corona characterisation, their use in nucleic acid-based biotechnology applications and more recently due to their promise as a proteomic enrichment tool.^{9,35,36,42}

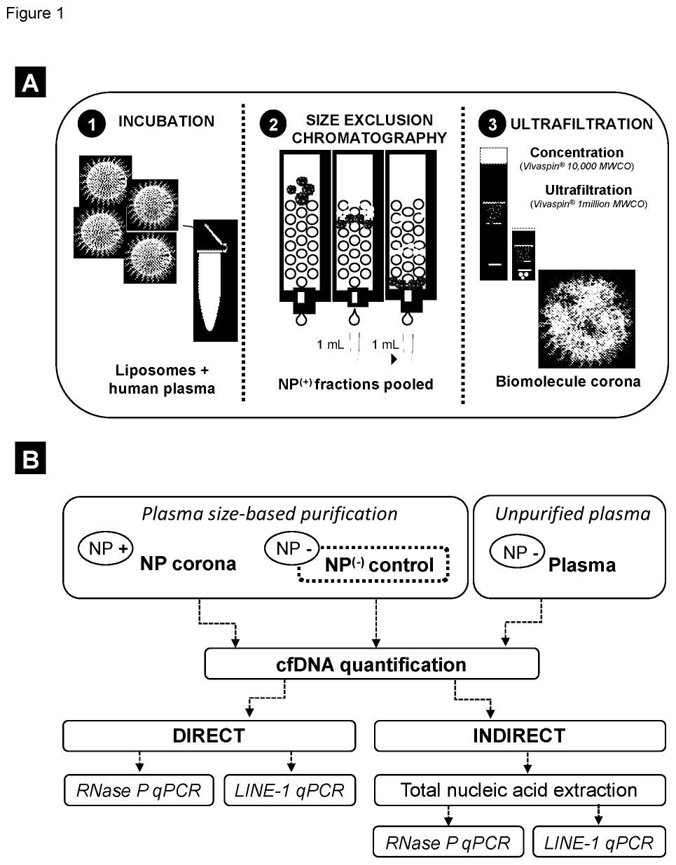

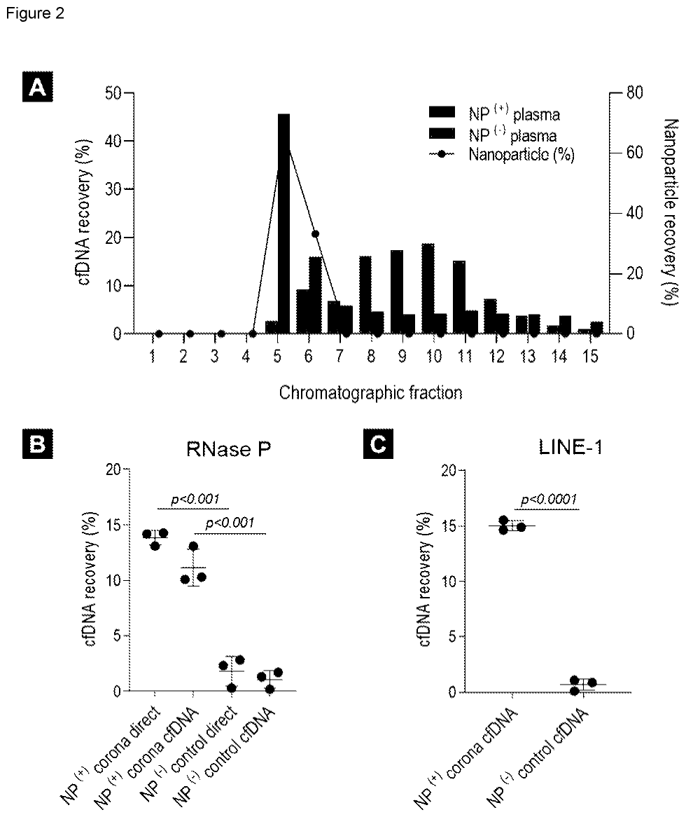

In order to assess the potential interaction of cfDNA with PEGylated liposomal surfaces, plasma-incubated liposomes were purified by size exclusion chromatography (SEC); represented in FIG. 1A), as described previously.14 Plasma control samples (without prior incubation with liposomes) were subjected to the exact same purification process. SEC column-eluted cfDNA was extracted from chromatographic fractions 1-15, using a QIAamp® circulating nucleic acid extraction kit (QIAGEN) and subsequently quantified using robust and highly sensitive LINE-1 real-time qPCR assay (FIG. 2A). Stewart assay was also performed in order to quantify the amount of liposomes eluted.

As illustrated in FIG. 2A and in agreement with our previous studies,¹⁴ corona-coated liposomes were eluted in chromatographic fractions 5 and 6, while no detectable lipid content was found in the fractionated plasma control. Distribution of cfDNA across chromatographic fractions 1-15 revealed significant differences between plasma-incubated liposomes and the matched plasma control. In the case of the plasma-incubated liposome sample the majority of cfDNA (45.8%) was eluted in chromatographic fraction 5, which also contained the largest population of liposome NPs (66.7%), while liposome-free fractions 7-15 contained relatively small quantities of cfDNA (<6%). In contrast, a normal distribution of cfDNA was evident in the fractionated plasma control, with the highest amount of cfDNA detected in fraction 10 (18.8%). Notably, in the absence of NPs, only 2.6% of the cfDNA content was detected in fraction 5. The striking difference in cfDNA distribution between corona-coated liposomes and the fractionated plasma control suggests that a significant proportion of cfDNA eluted in fraction 5 could be associated with the eluted liposomes.

Our data provide the first experimental evidence of the presence of cfDNA in the NP corona samples and show that the majority of cfDNA detected is associated with the surface of liposomes and is not passively co-eluted during purification (FIGS. 2A-C).

1.2 Quantitative Detection of cfDNA in the Liposome Corona.

To further purify corona-coated liposomes from any remaining protein complexes and/or unbound cfDNA, chromatographic fractions 5 and 6 were pooled, concentrated and subsequently washed three times using a membrane ultrafiltration column (Vivaspin®, 1 million MWCO). 8,9,11

To determine the total cfDNA content of the liposomal corona two different real-time qPCR assays were utilised, as outlined in FIG. 1B. A real-time qPCR approach was chosen as the concentration of cfDNA in blood commonly falls below the lower limit of detection for absorbance and fluorescence-based DNA quantification methods. Initially, a standardised TagMan® RNase P detection real-time qPCR assay (Applied Biosystems®) was used to quantify the cfDNA content of the biomolecule corona in healthy plasma samples. As illustrated in FIG. 2B, the concentration of cfDNA measured in the corona samples was significantly higher in comparison to plasma control samples that underwent the full purification process (adjusted p-value<0.0001). A small amount of cfDNA was identified in purified plasma controls, suggesting a co-elution of a small population of cfDNA molecules complexed with large proteins or within extracellular vesicles (FIG. 2B). These data suggested that most of the cfDNA quantified in corona samples is associated (directly or indirectly) with the surface of liposomes and was not passively co-eluted in a size-dependent manner.

In order to investigate whether the presence of proteins and/or other molecules in the biomolecule corona affects the direct quantification of cfDNA, we compared the amount of cfDNA with and without prior extraction (QIAGEN's QIAamp® circulating nucleic acid extraction kit). Comparable amounts of cfDNA were detected using the TaqMan® RNase P assay both in corona-coated liposome samples and in cfDNA subsequently purified from the same corona samples (FIG. 2B). These data indicated that the real-time qPCR assay was not significantly inhibited by other molecules present in the corona, allowing direct cfDNA measurements in the presence of lipid-based NPs and complex biofluid contaminants. To further investigate qPCR inhibition in NP-corona samples, a 2-fold dilution was performed prior to real-time qPCR quantification (FIGS. 3A&B). The cfDNA quantity of the 1:2 diluted corona sample was approximately half that of the original measurement (48%), providing further evidence to support the lack of RNase P qPCR inhibition in these direct real-time PCR measurements. The concentration of cfDNA in the NP-corona samples and plasma controls (with no NPs) was confirmed with a robust and sensitive LINE-1 qPCR assay (FIG. 2C). Both assays produced similar values, with RNase P and LINE-1 quantification methods consistently detecting significantly more cfDNA in corona samples when compared to plasma controls, as shown in FIG. 2C.

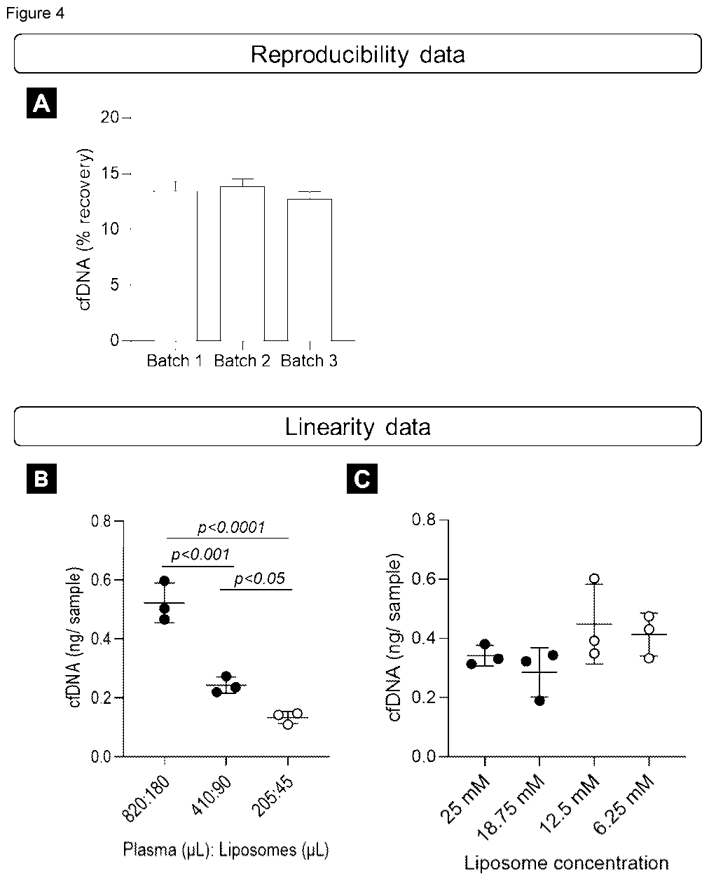

In terms of reproducibility, the percentage of cfDNA recovered with liposomal NPs was consistent across healthy plasma and liposome batches (FIG. 4A). In addition, plasma linearity experiments revealed a significant reduction in total cfDNA content when plasma input volume was lowered, while the plasma:NP ratio was maintained (adjusted p-values <0.01 for both 410 μL & 205 μL of plasma when compared to 810 μL) (FIG. 4B). In contrast to the linear relationship observed between plasma volume and cfDNA concentration, altering the concentration of liposome NPs did not significantly affect the amount cfDNA recovered (FIG. 3C). Combined, these data suggested that at the NP concentrations investigated, liposomes interacted reproducibly with a sub-population of plasma cfDNA molecules and that a NP:plasma [μL:μL] ratio of 0.2 was found optimal to recover this fraction of cfDNA.

Direct quantification of cfDNA was possible within complex lipid-based biomolecule corona samples without prior cfDNA extraction using the QIAamp circulating nucleic acid extraction kit (QIAGEN). In addition, cfDNA was successfully purified from lipid NPs using a standard cfDNA extraction kit, highlighting the compatibility of lipid-based NPs with downstream purification and quantification methods.

1.3 Detection of cfDNA in Ovarian Carcinoma Liposomal Corona Samples.

To establish whether cfDNA could also be detected on the surface of liposomes incubated ex vivo with plasma obtained from cancer patients, corona-coated liposomes were prepared upon incubation and purification from plasma samples obtained from 43 patients with ovarian cancer (18 patients with FIGO stage I, 8 with stage II, 12 with stage III and 5 with stage IV) (Table 1).

TABLE 1
Table outlining clinical characteristics of ovarian cancer patient cohort and healthy normal volunteers (HNVs). Details
include sample number (n), age-range (years), histological subtype, germline BRCA mutation status, baseline CA125
concentration (U/mL), prior lines of chemotherapy and platinum sensitivity.

| | Ovarian cancer patients | | | | | | | | |
|---|---|---|---|---|---|---|---|---|---|
| | Healthy | Stage 1 | | Stage 2 | | Stage 3 | | Stage 4 | |
| Sample number | 11 | 18 | | 8 | | 12 | | 5 | |
| Age-range (median) | 40-59 (51) | 21-87 | (59) | 32-77 | (60) | 37-74 | (62) | 36-67 | (48) |
| Histological subtype | N/A | Mucinous-11 | (61%) | Serous-6 | (75%) | Serous-9 | (75%) | Serous-5 | (100%) |
| | | Serous-5 | (28%) | Endometroid-2 | (25%) | Adenocarcinoma | (17%) | | |
| | | Clear cell-1 | (5.5%) | | | (NOS)-2 | | | |
| | | Endometroid-1 | (5.5%) | | | Carcinosarcoma-1 | (8%) | | |
| Germline BRCA | N/A | Positive-0 | (0%) | Positive-0 | (0%) | Positive-1 | (8%) | Positive-1 | (20%) |
| status | | Negative-1 | (5.5%) | Negative-3 | (37.5%) | Negative-0 | (0%) | Negative-3 | (60%) |
| | | Unknown-17 | (94.5%) | Unknown-5 | (62.5%) | Unknown-11 | (92%) | Unknown-1 | (20%) |
| Baseline CA125 | N/A | Median 60 | (12-550) | Median 29.5 | (4-600) | Median 16 | (7-358) | Median 15 | (9-396) |
| (U/mL) | | | | | | | | | |
| Prior lines of | N/A | 0 | (94%) | 0 | (62.5%) | 0 | (50%) | 0 | (20%) |
| chemotherapy | | 2 | (6%) | 1 | (37.5%) | 1 | (42%) | 1 | (80%) |
| | | | | | | 2 | (8%) | | |
| Platinum sensitivity | N/A | Sensitive-6 | (33%) | Sensitive-3 | (37.5%) | Sensitive-1 | (8%) | Sensitive-2 | (40%) |
| | | Resistant-1 | (6%) | Resistant-1 | (12.5%) | Resistant-0 | (0%) | Resistant-1 | (20%) |
| | | Unknown-11 | (61%) | Unknown-4 | (50%) | Unknown-11 | (92%) | Unknown-2 | (40%) |

Patients with ovarian cancer classified across all stages of the disease were included in the study to determine whether cfDNA could be detected in NP corona samples both at early stages and as the disease progressed. These samples were quantified directly using a robust high sensitivity LINE-1 qPCR assay and compared to corona samples from 11 healthy aged matched females (FIG. 5). When normalised to post-purification liposome concentration, cfDNA was significantly higher in ovarian cancer samples (all stages, early stage (I and II) and late-stage (III and IV)) compared to healthy controls (p values=<0.001, <0.01 and <0.0001, respectively) (FIG. 5). In addition, average cfDNA content increased from early (FIGO stage I and II) to late stage (FIGO stage III and IV), although this was not statistically significant (FIG. 5B). These data are consistent with previous studies that have proposed quantification cfDNA as a diagnostic and prognostic biomarker for ovarian cancer, with increased cfDNA levels detected with disease progression.^{43,44}

To determine whether direct cfDNA quantification in ovarian cancer corona samples would be inaccurate and skewed, with real-time qPCR inhibition increasing disproportionately with cancer stage, we compared cfDNA concentration in purified and unpurified samples for eight late-stage (stage III n=6, stage IV n=2) high-grade serous ovarian cancer samples (details provided in FIG. 3E). Similar cfDNA concentrations were measured for both unpurified ovarian cancer corona samples and their respective purified cfDNA samples (FIG. 3C). This suggests that real-time qPCR was not significantly inhibited in these biomolecule corona qPCR reactions and that no significant cfDNA loss occurred during cfDNA extraction using QIAGEN's QIAamp® circulating nucleic acid extraction kit. We were also able to measure the cfDNA content directly in ovarian cancer plasma samples (diluted 1:40), which again showed no significant difference from the respective purified plasma cfDNA samples (FIG. 3D).

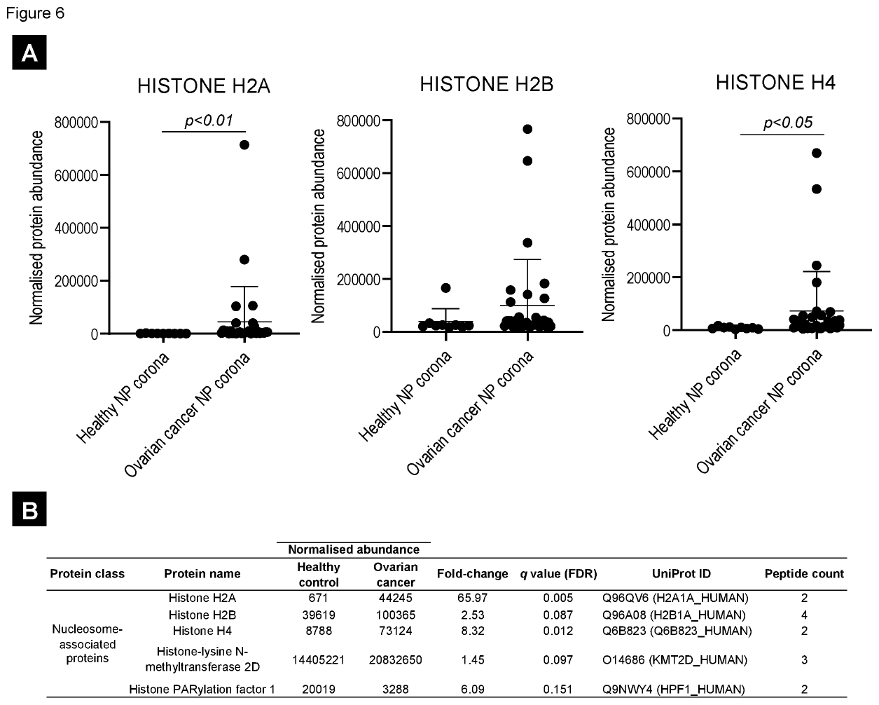

Mass spectrometry (LC-MS/MS) proteomic analysis was then performed on the 43 samples from ovarian cancer patients and the 11 samples from healthy controls to investigate whether proteins known to associate with cfDNA could be detected in the biomolecule corona (FIG. 6). Histone proteins, H2A, H2B and H4, which are found within the core nucleosome complex, were detected in the biomolecule corona and were identified at significantly higher levels in ovarian cancer samples relative to healthy controls (FIG. 6A). Two additional nucleosome-interacting proteins were identified in these samples, namely histone-lysine N-methyltransferase 2D and histone PARylation factor 1 (FIG. 6B)⁴⁵ Combined, these data confirmed the presence of cfDNA in the biomolecule corona of liposomes and suggested an indirect interaction which is potentially mediated via the nucleosome complex.

1.4

The PEGylated liposomes used in this study have a negative surface charge (FIG. 7A), therefore it was considered unlikely that DNA molecules would be bound directly onto the liposome surface via electrostatic interactions. Considering that cfDNA is protected within nucleosome complexes in the blood,⁴⁸ we hypothesised that cfDNA may not be directly bound onto the liposome surface, but through the adsorption of DNA-protein complexes. This indirect mechanism of adsorption was further supported by the identification of positively charged nucleosome core proteins, including histone proteins H2A, H2B and H4, in the biomolecule corona by LC-MS/MS analysis (FIG. 6). Of note, our group has previously detected histone proteins in human ex vivo, human in vivo and mouse in vivo liposomal corona samples.^{8,10,13} Moreover, human histone proteins (H2B and H4) have also been identified in the healthy corona of colloidal gold NPs.⁵² Furthermore, De Paoli and colleagues demonstrated that calf thymus histone H1 binds to carboxylated-multiwalled carbon nanotubes (CNTCOOH).⁵³ In addition, consistent cfDNA recovery across batches (FIG. 4A) suggested its reproducible and stable interaction with the liposomal surface as part of the biomolecule corona.

1.5 Discussion

Our data demonstrated that the corona-containing cfDNA levels were significantly higher in the biomolecule coronas formed upon incubation with plasma samples obtained from ovarian cancer patients (both early- and late-stages) in comparison to healthy controls (FIG. 5). It has been widely reported that total cfDNA is elevated in many different cancer types, such as colorectal, glioblastoma, colorectal and breast cancer, and increases with progression of the disease.^{44,54-57} It is important to clarify that DNA originating from the tumour frequently only makes up a small proportion of total cfDNA, with the majority of DNA molecules released from non-malignant cells.^{48,58} Moreover, healthy cfDNA detected in individuals with cancer is commonly of hematopoietic origin and can be attributed to increased white blood cell turnover and chemotherapeutic- and/or radiation-induced cell death.^{48,54} The elevated cfDNA detected in ovarian cancer patients in this study may therefore be attributable to cfDNA released from normal cells.

The ability to conduct genomic analysis on NP-corona offers up the ability to discover and analyze cancer-specific biomarkers in the NP corona. This approach could offer significant advantages over current purification methods, which lack the sensitivity required to detect ctDNA in small volumes of human plasma in patients with low tumour burden, especially pertinent to the challenge of early cancer detection.

Previous observations have shown that physiological diseased states affects blood composition, which is reflected in corona formation.⁸ For example, our group has previously shown that protein corona quantitatively and qualitatively changed in the presence of tumorigenesis, with higher total amount of protein found to interact with intravenously injected liposomes recovered from melanoma and lung adenocarcinoma tumour-bearing mice in comparison to healthy controls.⁸ Further analysis revealed that histone H2A was significantly upregulated in the in vivo lung adenocarcinoma corona samples.⁸ Therefore, the increased amount of nucleosome-related proteins in ovarian cancer samples is likely to extend to other cancer types and NP classes, as a general reflection of increased cfDNA and histone content, commonly seen in cancer.^{47,59-81} In terms of other pathological conditions, our previous analysis of the ex vivo corona formed in the plasma of sepsis patients revealed a significant increase in histone H2B compared to plasma from both systemic inflammatory response syndrome (SIRS) patients and healthy controls.¹⁰ Comprehensive comparison of ‘healthy’ and ‘diseased’ protein coronas has been found to be a very promising enrichment tool for plasma analysis, enabling proteomic discovery of low abundant, diagnostic biomarkers.^{9,10}

In recent years, other cell-free nucleic acids, such as miRNAs, have received growing interest as disease biomarkers⁶² and although extensive characterisation of the NP corona nucleic acid content was beyond the scope of this study, it remains an important avenue of future research. In addition, epigenetic analysis of ctDNA, such as differential methylation profiles can also provide cancer-specific signatures.⁶³ Intriguingly, methyl-cytosines have been shown to display a strong affinity to bare metal surfaces, including gold nanoparticles.^{64,65} Furthermore, post translational modifications of histone proteins have also been widely associated with tumourigenesis and have been previously detected in the plasma of cancer patients.⁶⁸⁻⁷¹ The molecular complexes of cell-free nucleic acids contained with the biomolecule corona need to be fully elucidated in order to establish the scope for a sensitive blood-based biomarker enrichment tool.

The molecular information contained within the NP corona is far richer than originally described and has been shown to contain a diverse array of biomolecules including proteins, lipids, metabolites and now cfDNA. This complex coating on the surface of NPs has the potential to be able to enhance nano-drug delivery and NP uptake, but perhaps most significantly, offers the potential to provide greater sensitivity for liquid biopsies.

This study has shown that cell-free DNA is present in the biomolecule corona that forms around lipid-based NPs, upon incubation with human plasma. The cfDNA content of the biomolecule corona could be directly quantified in the presence other biomolecules (e.g. proteins) using conventional real-time qPCR assays. Furthermore, proteomic analysis of the biomolecule corona by LC-MS/MS revealed the presence of nucleosome complex proteins, suggesting an indirect protein-mediated interaction of cfDNA with NPs. Notably, the amount of cfDNA was found to be significantly higher in the coronas formed in early- and late-stage cancer patient plasma samples compared to healthy controls, indicating a disease-specific biomolecule corona formation. This study highlights the potential exploitation of the biomolecule corona as a novel blood-analysis nanoscale tool and that multi-omic analysis can be carried out on the NP-corona, such as from a single sample, either sequentially or in parallel.

### Example 2. A Multi-Omic Approach

Ongoing biomarker development efforts indicate that multiple markers, used individually or as part of a panel, are required to provide sufficient sensitivity and specificity for early disease detection. In addition, understanding the heterogeneous underlying mechanisms requires the integration of multiple omics approaches. Examining molecular alterations in blood at multiple dimensions (genome, proteome, metabolome etc.) and integrating the resultant multi-omics data not only has the potential to elucidate disease-specific molecular mechanisms and pathways, but also to uncover novel biomarkers to aid early disease detection, patient stratification and disease monitoring (Cohen J D et al., Science, 2018, 359,926-930; Hristova V A, Chan D W, Expert Rev Proteomics, 2019; 16(2)93-103).

Currently, one of the major bottlenecks for the multi-omics analysis of blood is the large volume of patient sample required (˜10-15 ml), in order to distinctly enrich and extract proteins, nucleic acids and lipids. This not only limits analytical reproducibility, but it also compromises the comparability of the resultant omics data sets. The minimally invasive blood collection procedures, coupled with the ability to perform integrative multi-omics analysis on a single specimen are tremendous advantages that could redefine the future of biomarker discovery. (Hristova V A, Chan D W, Expert Rev Proteomics, 2019; 16(2)93-103).

2.1 The NP-biomolecule coronas produced from the subjects in Example 1 were subjected to multi-omic analysis (genomic, proteomic and lipidomic) as described in the Materials and methods.

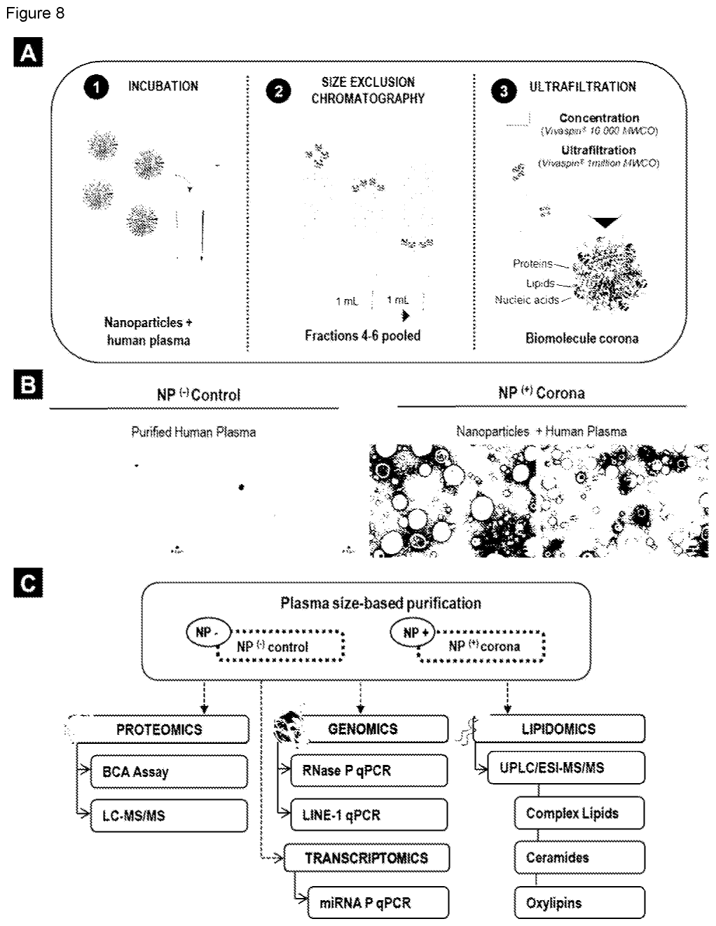

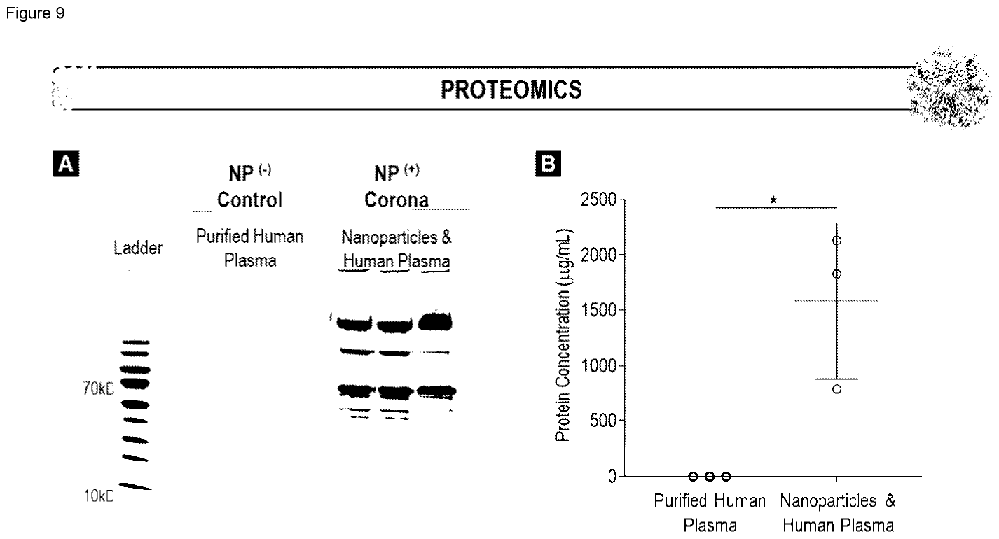

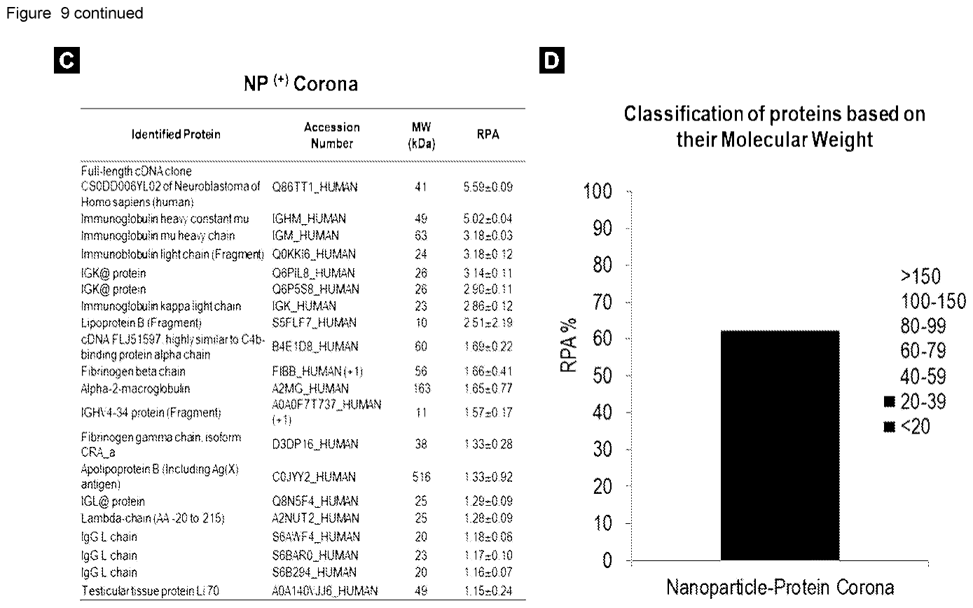

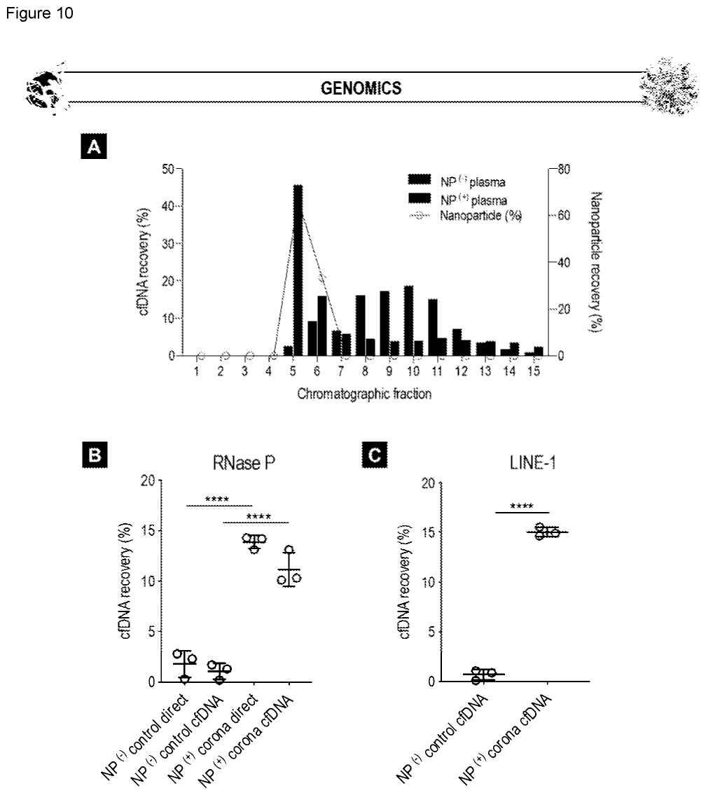

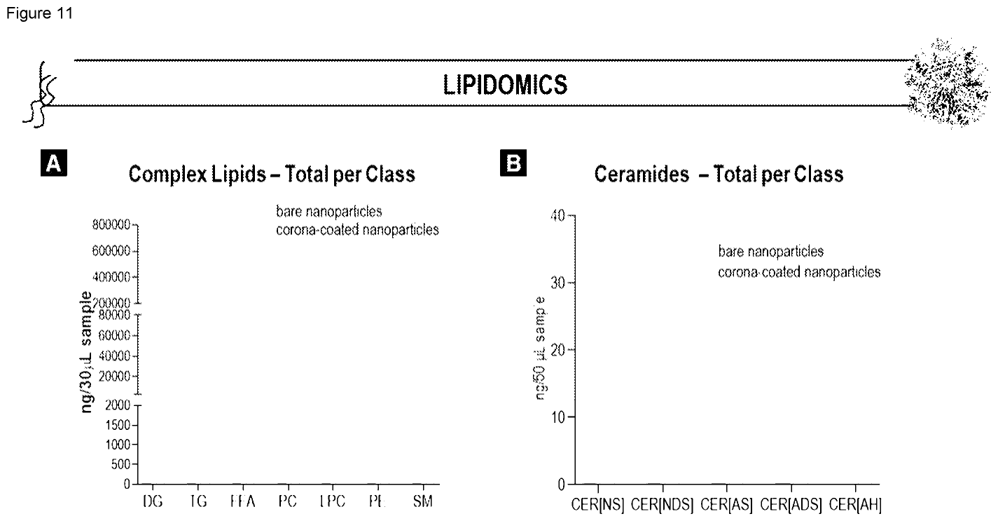

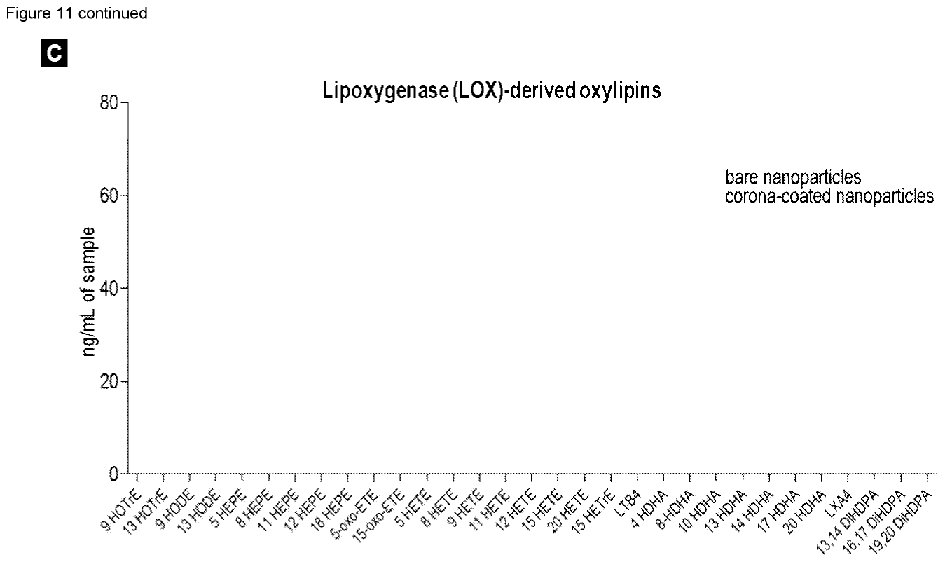

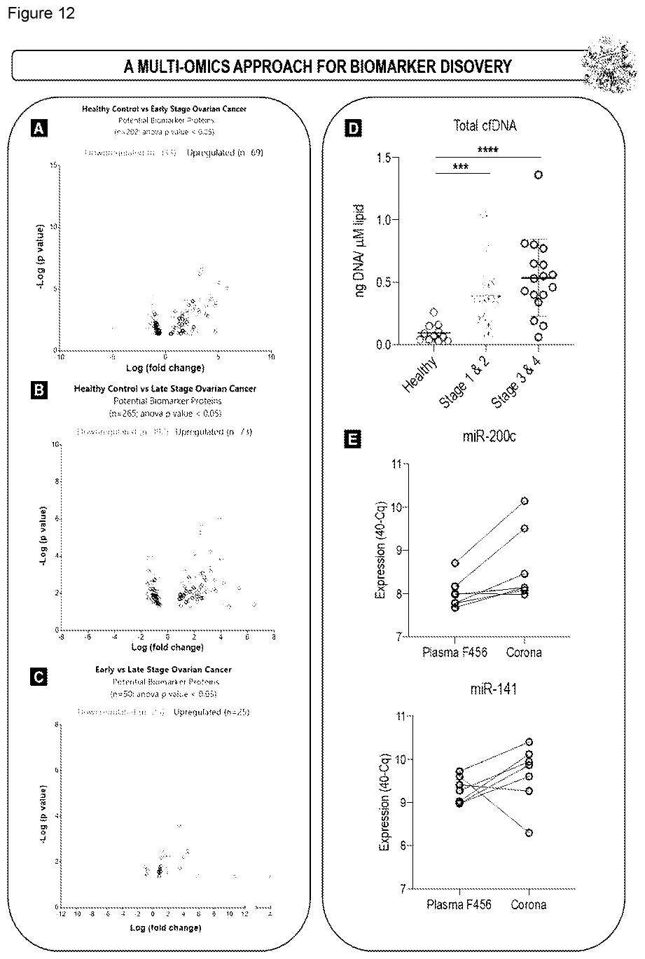

The data generated is shown in FIGS. 8-12 and in Tables 2-8 below. This demonstrates that a single processed sample can be subjected to multi-omic analysis. Analyzing a single sample source will facilitate more accurate comparison of data.

TABLE 2
Mass Spectrometry-based proteomic analysis. Full list of proteins identified by Scaffold
Software tool in healthy human plasma and onto the surface of PEG:HSPC:CHOL liposomes
classified from the highest relative protein abundance (RPA) to the lowest.

| | | | RPA NP- | STDV NP- |
|---|---|---|---|---|
| | Accession | MW | Protein | Protein |
| Identified Proteins (n = 315) | Number | (kDa) | Corona | Corona |
| Full-length cDNA clone CS0DD006YL02 of | Q86TT1_HUMAN | 41 | 5.59 | 0.09 |
| Neuroblastoma of Homo sapiens (human) | | | | |
| Immunoglobulin heavy constant mu | IGHM_HUMAN | 49 | 5.02 | 0.04 |
| GN = IGHM | | | | |
| Lipoprotein B (Fragment) GN = APOB | S5FLF7_HUMAN | 10 | 2.51 | 2.19 |
| Immunoblobulin light chain (Fragment) | Q0KKI6_HUMAN | 24 | 3.18 | 0.12 |
| IGK@ protein | Q6PIL8_HUMAN | 26 | 3.14 | 0.11 |
| Immunoglobulin mu heavy chain | IGM_HUMAN | 63 | 3.18 | 0.03 |
| IGK@ protein | Q6P5S8_HUMAN | 26 | 2.90 | 0.11 |
| Immunoglobulin kappa light chain | IGK_HUMAN | 23 | 2.86 | 0.12 |
| Alpha-2-macroglobulin GN = A2M | A2MG_HUMAN | 163 | 1.65 | 0.77 |
| Fibrinogen beta chain GN = FGB | FIBB_HUMAN (+1) | 56 | 1.66 | 0.41 |
| Apolipoprotein B (Including Ag(X) antigen) | C0JYY2_HUMAN | 516 | 1.33 | 0.92 |
| GN = APOB | | | | |
| cDNA FLJ51597, highly similar to C4b- | B4E1D8_HUMAN | 60 | 1.69 | 0.22 |
| binding protein alpha chain | | | | |
| Fibrinogen gamma chain, isoform CRA_a | D3DP16_HUMAN | 38 | 1.33 | 0.28 |
| GN = FGG | | | | |
| IGHV4-34 protein (Fragment) | A0A0F7T737_HUMAN | 11 | 1.57 | 0.17 |
| GN = IGHV4-34 | (+1) | | | |
| Testicular tissue protein Li 70 | A0A140VJJ6_HUMAN | 49 | 1.15 | 0.24 |
| IgG L chain | S6AWF4_HUMAN | 20 | 1.18 | 0.06 |
| IgG L chain | S6B294_HUMAN | 20 | 1.16 | 0.07 |
| IGL@ protein | Q8N5F4_HUMAN | 25 | 1.29 | 0.09 |
| Lambda-chain (AA −20 to 215) | A2NUT2_HUMAN | 25 | 1.28 | 0.09 |
| IGL@ protein | Q6PIQ7_HUMAN | 25 | 0.39 | 0.68 |
| IgG L chain | S6BAR0_HUMAN | 23 | 1.17 | 0.10 |
| IGL@ protein | Q6PIK1_HUMAN | 25 | 1.11 | 0.03 |
| IgG L chain | S6AWE6_HUMAN | 23 | 1.13 | 0.10 |
| Fibrinogen alpha chain GN = FGA | FIBA_HUMAN | 95 | 0.90 | 0.16 |
| Uncharacterized protein | Q8NEJ1_HUMAN | 25 | 1.08 | 0.10 |
| IGL@ protein | Q5FWF9_HUMAN | 25 | 1.11 | 0.14 |
| 10E8 heavy chain variable region | A0A193CHQ9_HUMAN | 14 | 1.07 | 0.20 |
| (Fragment) | | | | |
| Anti-FactorVIII scFv (Fragment) | A2KBC6_HUMAN | 25 | 0.93 | 0.08 |
| Apolipoprotein E isoform 1 (Fragment) | A0A0S2Z3D5_HUMAN | 36 | 0.98 | 0.11 |
| | (+1) | | | |
| Myosin-reactive immunoglobulin heavy | Q9UL90_HUMAN | 12 | 0.95 | 0.17 |
| chain variable region (Fragment) | | | | |
| GCT-A1 heavy chain variable region | A0A125U0V2_HUMAN | 14 | 1.01 | 0.20 |
| (Fragment) | | | | |
| Haptoglobin-related protein GN = HPR | HPTR_HUMAN | 39 | 0.94 | 0.12 |
| Anti-streptococcal/anti-myosin | Q96SA9_HUMAN | 12 | 0.83 | 0.04 |
| immunoglobulin kappa light chain | | | | |
| variable region (Fragment) | | | | |
| Rheumatoid factor RF-ET6 (Fragment) | A2J1N5_HUMAN | 10 | 0.76 | 0.14 |
| GCT-A5 light chain variable region | A0A0X9UWL5_HUMAN | 12 | 0.72 | 0.11 |
| (Fragment) | | | | |
| Immunoglobulin heavy variable 3-74 | HV374_HUMAN | 13 | 0.79 | 0.03 |
| GN = IGHV3-74 | | | | |
| Apolipoprotein A-I, isoform CRA_a | A0A024R3E3_HUMAN | 31 | 0.80 | 0.04 |
| N = APOA1 | (+1) | | | |
| Myosin-reactive immunoglobulin heavy | Q9UL88_HUMAN | 14 | 0.47 | 0.40 |
| chain variable region (Fragment) | | | | |
| A30 (Fragment) | A2MYE1_HUMAN (+1) | 10 | 0.67 | 0.02 |
| GCT-A4 light chain variable region | A0A0X9T7V9_HUMAN | 12 | 0.73 | 0.13 |
| (Fragment) | | | | |
| CD5 antigen-like GN = CD5L | CD5L_HUMAN | 38 | 0.68 | 0.06 |
| Haptoglobin GN = HP | HPT_HUMAN (+2) | 45 | 0.69 | 0.06 |
| Protein S isoform 1 (Fragment) | A0A0S2Z4K3_HUMAN | 75 | 0.62 | 0.02 |
| GN = PROS1 | (+2) | | | |
| Apolipoprotein D GN = APOD | APOD_HUMAN (+1) | 21 | 0.55 | 0.05 |
| IGH@ protein GN = IGH@ | Q6GMX6_HUMAN | 51 | 0.64 | 0.05 |
| Epididymis luminal protein 214 | V9HW68_HUMAN | 52 | 0.58 | 0.03 |
| GN = HEL-214 | | | | |
| GCT-A6 heavy chain variable region | A0A109PVK5_HUMAN | 15 | 0.53 | 0.06 |
| (Fragment) | | | | |
| Myosin-reactive immunoglobulin light | Q9UL83_HUMAN | 12 | 0.53 | 0.02 |
| chain variable region (Fragment) | | | | |
| Immunglobulin heavy chain variable | Q0ZCI6_HUMAN | 14 | 0.37 | 0.33 |
| region (Fragment) | | | | |
| Variable immnoglobulin anti-estradiol | A2NZ55_HUMAN | 14 | 0.18 | 0.31 |
| heavy chain (Fragment) | | | | |
| Complement C3 GN = C3 | CO3_HUMAN (+1) | 187 | 0.48 | 0.03 |
| IGHV3-72 protein (Fragment) | A0A0F7TAG7_HUMAN | 12 | 0.53 | 0.05 |
| GN = IGHV3-72 | (+1) | | | |
| Myosin-reactive immunoglobulin light | Q9UL70_HUMAN | 12 | 0.47 | 0.04 |
| chain variable region (Fragment) | | | | |
| Serum albumin GN = ALB | ALBU_HUMAN | 69 | 0.52 | 0.03 |
| cDNA FLJ14473 fis, clone | Q96K68_HUMAN | 53 | 0.57 | 0.09 |
| MAMMA1001080, highly similar to | | | | |
| Homo sapiens SNC73 protein (SNC73) | | | | |
| mRNA | | | | |
| Uncharacterized protein | Q6MZX9_HUMAN | 52 | 0.48 | 0.01 |
| DKFZp686M08189 | | | | |
| GN = DKFZp686M08189 | | | | |
| Single-chain Fv (Fragment) GN = scFv | Q65ZC9_HUMAN | 26 | 0.16 | 0.27 |
| Uncharacterized protein | A8K008_HUMAN | 52 | 0.51 | 0.04 |
| MS-D4 heavy chain variable region | A0A0X9UWK7_HUMAN | 14 | 0.48 | 0.02 |
| (Fragment) | | | | |
| Complement component 1, q | A0A024RAB9_HUMAN | 27 | 0.45 | 0.01 |
| subcomponent, B chain, isoform | (+3) | | | |
| CRA_a GN = C1QB | | | | |
| cDNA FLJ41981 fis, clone | Q6ZVX0_HUMAN | 53 | 0.46 | 0.02 |
| SMINT2011888, highly similar to | | | | |
| Protein Tro alpha1 H, myeloma | | | | |
| Rheumatoid factor RF-ET9 (Fragment) | A2J1N6_HUMAN | 13 | 0.52 | 0.05 |
| Immunoglobulin heavy variable 3-73 | HV373_HUMAN | 13 | 0.41 | 0.06 |
| GN = IGHV3-73 | | | | |
| Immunoglobulin heavy chain variant | Q9NPP6_HUMAN | 45 | 0.47 | 0.04 |
| (Fragment) | | | | |
| IgG H chain | S6B291_HUMAN | 51 | 0.47 | 0.03 |
| Uncharacterized protein GN = | Q6N089_HUMAN | 52 | 0.45 | 0.02 |
| DKFZp686P15220 | | | | |
| Cold agglutinin FS-1 L-chain (Fragment) | A2NB45_HUMAN | 12 | 0.41 | 0.09 |
| Immunoglobulin alpha-2 heavy chain | IGA2_HUMAN | 49 | 0.40 | 0.01 |
| Apolipoprotein C-III GN = APOC3 | A3KPE2_HUMAN (+2) | 11 | 0.39 | 0.02 |
| IBM-B2 heavy chain variable region | A0A125QYY9_HUMAN | 14 | 0.43 | 0.07 |
| (Fragment) | | | | |
| Ig heavy chain variable region | A0A068LKQ2_HUMAN | 13 | 0.47 | 0.11 |
| (Fragment) | | | | |
| Immunoglobulin heavy variable 1-2 | HV102_HUMAN | 13 | 0.38 | 0.01 |
| GN = IGHV1-2 | | | | |
| Clusterin GN = CLU | CLUS_HUMAN | 52 | 0.32 | 0.06 |
| N90-VRC38.08 heavy chain variable | A0A1W6IYI5_HUMAN | 14 | 0.38 | 0.05 |
| region (Fragment) | | | | |
| Alpha-1-antitrypsin GN = SERPINA1 | A0A024R6I7_HUMAN | 47 | 0.30 | 0.07 |
| Cryocrystalglobulin CC1 heavy chain | B1N7B6_HUMAN | 13 | 0.35 | 0.02 |
| variable region (Fragment) | | | | |
| Immunoglobulin heavy variable 3-13 | HV313_HUMAN | 13 | 0.31 | 0.05 |
| GN = IGHV3-13 | | | | |
| Immunoglobulin heavy variable 3-49 | HV349_HUMAN | 13 | 0.27 | 0.06 |
| GN = IGHV3-49 | | | | |
| Immunoglobulin heavy variable 1-46 | HV146_HUMAN | 13 | 0.26 | 0.06 |
| GN = IGHV1-46 | | | | |
| Immunoglobulin heavy variable 3-43 | HV343_HUMAN | 13 | 0.11 | 0.19 |
| GN = IGHV3-43 | | | | |
| Apolipoprotein C-IV GN = APOC4 | A5YAK2_HUMAN | 15 | 0.27 | 0.06 |
| cDNA, FLJ94213, highly similar to | B2R950_HUMAN (+1) | 164 | 0.23 | 0.11 |
| Homo sapiens pregnancy-zone protein | | | | |
| (PZP), mRNA | | | | |
| Cryocrystalglobulin CC1 kappa light | B1N7B8_HUMAN | 12 | 0.35 | 0.05 |
| chain variable region (Fragment) | | | | |
| Apolipoprotein M GN = APOM | APOM_HUMAN | 21 | 0.28 | 0.03 |
| Apolipoprotein C-I, isoform CRA_a | A0A024R0T8_HUMAN | 9 | 0.29 | 0.01 |
| GN = APOC1 | (+2) | | | |
| VH6DJ protein (Fragment) GN = VH6DJ | A2N0T9_HUMAN | 13 | 0.34 | 0.05 |
| Immunoglobulin kappa variable 1-8 | KV108_HUMAN | 13 | 0.31 | 0.02 |
| GN = IGKV1-8 | | | | |
| Lectin galactoside-binding soluble 3 | A0A0S2Z3Y1_HUMAN | 65 | 0.26 | 0.02 |
| binding protein isoform 1 (Fragment) | (+1) | | | |
| GN = LGALS3BP | | | | |
| Complement C4-B GN = C4B | CO4B_HUMAN | 193 | 0.25 | 0.03 |
| Immunoglobulin J chain GN = JCHAIN | IGJ_HUMAN | 18 | 0.31 | 0.07 |
| Complement component 1, q | A0A024RAA7_HUMAN | 26 | 0.26 | 0.01 |
| subcomponent, C chain, isoform CRA_a | (+1) | | | |
| GN = C1QC | | | | |
| VH6DJ protein (Fragment) GN = VH6DJ | A2N0U5_HUMAN | 12 | 0.25 | 0.02 |
| V1-2 protein (Fragment) GN = V1-2 | A2MYD6_HUMAN | 10 | 0.17 | 0.15 |
| Complement C4-A GN = C4A | A0A0G2JPR0_HUMAN | 193 | 0.24 | 0.03 |
| Uncharacterized protein | Q6MZU6_HUMAN | 51 | 0.30 | 0.04 |
| DKFZp686C15213 | | | | |
| GN = DKFZp686C15213 | | | | |
| Immunoglobulin heavy variable 2-70D | HV70D_HUMAN | 13 | 0.29 | 0.03 |
| GN = IGHV2-70D | | | | |
| Apolipoprotein A-II GN = APOA2 | APOA2_HUMAN (+3) | 11 | 0.22 | 0.02 |
| MS-A2 light chain variable region | A0A0X9V981_HUMAN | 11 | 0.22 | 0.02 |
| (Fragment) | | | | |
| cDNA FLJ75066, highly similar to | A8K5J8_HUMAN | 80 | 0.20 | 0.04 |
| Homo sapiens complement component | | | | |
| 1, r subcomponent (C1R), mRNA | | | | |
| V1-3 protein (Fragment) GN = V1-3 | Q5NV84_HUMAN | 10 | 0.21 | 0.01 |
| APOC4-APOC2 readthrough (NMD | K7ER74_HUMAN | 20 | 0.20 | 0.02 |
| candidate) GN = APOC4-APOC2 | | | | |
| Anti-Influenza A hemagglutinin heavy | G1FM90_HUMAN | 15 | 0.20 | 0.03 |
| chain variable region (Fragment) | | | | |
| APOL1 protein (Fragment) GN = APOL1 | A5PL32_HUMAN (+4) | 49 | 0.19 | 0.02 |
| Uncharacterized protein | Q6N030_HUMAN | 57 | 0.23 | 0.02 |
| GN = DKFZp686I15212 | | | | |
| Immunoglobulin heavy variable 1-18 | HV118_HUMAN | 13 | 0.23 | 0.04 |
| GN = IGHV1-18 | | | | |
| GCT-A5 heavy chain variable region | A0A0X9T0H6_HUMAN | 13 | 0.22 | 0.01 |
| (Fragment) | | | | |
| Immunoglobulin kappa variable 2D-29 | KVD29_HUMAN | 13 | 0.14 | 0.12 |
| GN = IGKV2D-29 | | | | |
| cDNA, FLJ93914, highly similar to | B2R8I2_HUMAN | 60 | 0.20 | 0.01 |
| Homo sapiens histidine-rich glycoprotein | | | | |
| (HRG), mRNA | | | | |
| Rheumatoid factor RF-IP12 (Fragment) | A2J1M8_HUMAN | 11 | 0.15 | 0.13 |
| Actin, alpha cardiac muscle 1 | ACTC_HUMAN | 42 | 0.21 | 0.03 |
| GN = ACTC1 | | | | |
| Serum paraoxonase/arylesterase 1 | PON1_HUMAN | 40 | 0.19 | 0.01 |
| GN = PON1 | | | | |
| SAA2-SAA4 readthrough | A0A096LPE2_HUMAN | 23 | 0.16 | 0.06 |
| GN = SAA2-SAA4 | | | | |
| Complement component 1, q | A0A024RAG6_HUMAN | 26 | 0.18 | 0.02 |
| subcomponent, A chain, isoform | (+1) | | | |
| CRA_a GN = C1QA | | | | |
| Complement factor H GN = CFH | CFAH_HUMAN | 139 | 0.12 | 0.09 |
| Amyloid lambda 6 light chain variable | Q96JD1_HUMAN | 12 | 0.21 | 0.02 |
| region PIP (Fragment) | | | | |
| C4b-binding protein beta chain | C4BPB_HUMAN | 28 | 0.18 | 0.03 |
| GN = C4BPB | | | | |
| Fibronectin 1, isoform CRA_n | A0A024R462_HUMAN | 259 | 0.12 | 0.10 |
| GN = FN1 | (+1) | | | |
| Immunoglobulin kappa variable 1-16 | KV116_HUMAN | 13 | 0.19 | 0.04 |
| GN = IGKV1-16 | | | | |
| Immunoglobulin heavy variable 3-64D | HV64D_HUMAN | 13 | 0.16 | 0.01 |
| GN = IGHV3-64D | | | | |
| Immunoglobulin kappa variable 2D-24 | A0A075B6R9_HUMAN | 13 | 0.16 | 0.01 |
| (non-functional) (Fragment) GN = | (+1) | | | |
| IGKV2D-24 | | | | |
| Immunoglobulin heavy variable 3-64 | HV364_HUMAN | 13 | 0.15 | 0.03 |
| GN = IGHV3-64 | | | | |
| Immunoglobulin heavy variable 2-26 | HV226_HUMAN | 13 | 0.14 | 0.03 |
| GN = IGHV2-26 | | | | |
| V1-13 protein (Fragment) GN = V1-13 | Q5NV69_HUMAN | 10 | 0.18 | 0.02 |
| Apolipoprotein A-IV GN = APOA4 | APOA4_HUMAN | 45 | 0.18 | 0.03 |
| V5-2 protein (Fragment) GN = V5-2 | A2MYC8_HUMAN (+2) | 11 | 0.19 | 0.07 |
| Ficolin-3 GN = FCN3 | FCN3 HUMAN | 33 | 0.15 | 0.00 |
| cDNA FLJ53075, highly similar to | B4DPP8_HUMAN (+1) | 46 | 0.13 | 0.01 |
| Kininogen-1 | | | | |
| Immunoglobulin kappa variable 6-21 | KV621_HUMAN | 12 | 0.22 | 0.08 |
| GN = IGKV6-21 | | | | |
| Immunoglobulin heavy constant gamma | A0A286YFJ8_HUMAN | 44 | 0.15 | 0.02 |
| 4 (Fragment) GN = IGHG4 | (+1) | | | |
| ADP/ATP translocase 3 GN = | ADT3_HUMAN (+2) | 33 | 0.10 | 0.03 |
| SLC25A6 | | | | |
| Ig heavy chain variable region | A0A068LN03_HUMAN | 13 | 0.19 | 0.06 |
| (Fragment) | | | | |
| Immunoglobulin lambda variable 8-61 | LV861_HUMAN (+1) | 13 | 0.15 | 0.04 |
| GN = IGLV8-61 PE = 3 SV = 7 | | | | |
| Prenylcysteine oxidase 1 GN = PCYOX1 | PCYOX_HUMAN | 57 | 0.13 | 0.01 |
| Transferrin variant (Fragment) | Q53H26_HUMAN | 77 | 0.13 | 0.02 |
| Lipoprotein, Lp(A) GN = LPA | Q1HP67_HUMAN | 227 | 0.07 | 0.06 |
| N90-VRC38.10 heavy chain variable | A0A1W6IYI8_HUMAN | 14 | 0.13 | 0.02 |
| region (Fragment) | | | | |
| N90-VRC38.05 heavy chain variable | A0A1W6IYJ2_HUMAN | 14 | 0.06 | 0.06 |
| region (Fragment) | | | | |
| cDNA FLJ56954, highly similar to | B7Z539_HUMAN | 72 | 0.07 | 0.06 |
| Inter-alpha-trypsin inhibitor heavy | | | | |
| chain H1 | | | | |
| Angiotensinogen GN = AGT | ANGT_HUMAN (+6) | 53 | 0.11 | 0.00 |
| Ficolin-2 GN = FCN2 | FCN2_HUMAN | 34 | 0.12 | 0.02 |
| Proteoglycan 4, isoform CRA_a | A0A024R930_HUMAN | 151 | 0.07 | 0.04 |
| GN = PRG4 | (+2) | | | |
| V4-2 protein (Fragment) GN = V4-2 | Q5NV82_HUMAN | 11 | 0.07 | 0.06 |
| Polymeric immunoglobulin receptor | PIGR_HUMAN | 83 | 0.11 | 0.01 |
| GN = PIGR | | | | |
| Protein AMBP GN = AMBP | AMBP_HUMAN | 39 | 0.07 | 0.03 |
| Phosphatidylinositol-glycan-specific | PHLD_HUMAN | 92 | 0.08 | 0.01 |
| phospholipase D GN = GPLD1 | | | | |
| Inter-alpha (Globulin) inhibitor H2 | A2RTY6_HUMAN (+3) | 106 | 0.05 | 0.05 |
| GN = ITIH2 | | | | |
| Actin, cytoplasmic 1 GN = ACTB | ACTB_HUMAN (+2) | 42 | 0.11 | 0.04 |
| Alpha-crystallin B chain GN = CRYAB | A0A024R3B9_HUMAN | 12 | 0.06 | 0.05 |
| | (+7) | | | |
| Serum amyloid P-component GN = | SAMP_HUMAN (+1) | 25 | 0.09 | 0.01 |
| APCS | | | | |
| Complement factor properdin isoform 1 | A0A0S2Z4I5_HUMAN | 51 | 0.08 | 0.01 |
| (Fragment) GN = CFP | (+1) | | | |
| von Willebrand factor GN = VWF | VWF_HUMAN | 309 | 0.06 | 0.04 |
| Immunoglobulin heavy variable 2-5 | HV205_HUMAN | 13 | 0.06 | 0.05 |
| GN = IGHV2-5 | | | | |
| HCG2039812, isoform CRA_b (Fragment) | A0A0S2Z428_HUMAN | 60 | 0.08 | 0.00 |
| GN = KRT6A | (+2) | | | |
| Transthyretin GN = TTR | A0A087WT59_HUMAN | 20 | 0.08 | 0.02 |
| | (+3) | | | |
| Vitronectin GN = VTN | D9ZGG2_HUMAN (+1) | 54 | 0.09 | 0.04 |
| Coagulation factor XI GN = F11 | FA11_HUMAN | 70 | 0.07 | 0.03 |
| IgGFc-binding protein GN = FCGBP | FCGBP_HUMAN | 572 | 0.09 | 0.02 |
| Coagulation factor V GN = F5 | A0A0A0MRJ7_HUMAN | 252 | 0.04 | 0.03 |
| | (+1) | | | |
| Complement C1s subcomponent GN = C1S | C1S_HUMAN | 77 | 0.05 | 0.02 |
| Alpha-2-antiplasmin GN = SERPINF2 | A2AP_HUMAN | 55 | 0.07 | 0.01 |
| Epididymis tissue protein Li 173 | E9KL26_HUMAN | 55 | 0.05 | 0.02 |
| GN = SERPING1 | (+1) | | | |
| Serpin peptidase inhibitor, clade A (Alpha- | A0A024R6P0_HUMAN | 48 | 0.09 | 0.02 |
| 1 antiproteinase, antitrypsin), member | (+2) | | | |
| 3, isoform CRA_c GN = SERPINA3 | | | | |
| Alpha-1-acid glycoprotein 2 GN = ORM2 | A1AG2_ HUMAN | 24 | 0.04 | 0.03 |
| Lipopolysaccharide-binding protein | LBP_HUMAN (+1) | 53 | 0.06 | 0.00 |
| GN = LBP | | | | |
| CP protein GN = CP | A5PL27_HUMAN (+5) | 122 | 0.05 | 0.01 |
| cDNA FLJ76342, highly similar to | A8K1K1_HUMAN (+1) | 57 | 0.06 | 0.00 |
| Homo sapiens carnosine dipeptidase 1 | | | | |
| (metallopeptidase M20 family) | | | | |
| (CNDP1), mRNA | | | | |
| Platelet-activating factor acetylhydrolase | A0A024RD39_HUMAN | 50 | 0.04 | 0.01 |
| GN = PLA2G7 | (+1) | | | |
| Anoctamin (Fragment) GN = ANO7 | H7C220_HUMAN | 21 | 0.02 | 0.03 |
| PE = 3 SV = 8 | | | | |
| Serpin peptidase inhibitor, clade C | A0A024R944_HUMAN | 53 | 0.05 | 0.00 |
| (Antithrombin), member 1, isoform CRA_a | (+2) | | | |
| GN = SERPINC1 | | | | |
| ATP synthase subunit alpha, mitochondrial | ATPA_HUMAN (+1) | 60 | 0.03 | 0.01 |
| GN = ATP5A1 | | | | |
| Adiponectin GN = ADIPOQ | A8K660_HUMAN (+2) | 26 | 0.03 | 0.03 |
| Carboxypeptidase N catalytic chain | CBPN_HUMAN | 52 | 0.02 | 0.02 |
| GN = CPN1 | | | | |
| Mannan-binding lectin serine protease | MASP1_HUMAN | 79 | 0.04 | 0.01 |
| 1 GN = MASP1 | | | | |
| cDNA FLJ77947, highly similar to | A8K9M5_HUMAN (+6) | 67 | 0.03 | 0.01 |
| Human complement protein C8 beta | | | | |
| subunit mRNA | | | | |
| Complement C5 GN = C5 | CO5_HUMAN | 188 | 0.04 | 0.00 |
| Soluble scavenger receptor cysteine-rich | SRCRL_HUMAN | 166 | 0.02 | 0.02 |
| domain-containing protein SSC5D | | | | |
| GN = SSC5D | | | | |
| Inter-alpha (Globulin) inhibitor H4 | B2RMS9_HUMAN (+1) | 103 | 0.03 | 0.01 |
| (Plasma Kallikrein-sensitive | | | | |
| glycoprotein) GN = ITIH4 | | | | |
| Adipocyte plasma membrane-associated | APMAP_HUMAN (+1) | 46 | 0.03 | 0.01 |
| protein GN = APMAP | | | | |
| Complement component 9, isoform | A0A024R035_HUMAN | 63 | 0.05 | 0.02 |
| CRA_a GN = C9 | (+1) | | | |
| Prothrombin GN = F2 | E9PIT3_HUMAN (+1) | 65 | 0.05 | 0.03 |
| ATPase Ca++ transporting cardiac | A0A0S2Z3L_2_HUMAN | 115 | 0.03 | 0.00 |
| muscle slow twitch 2 isoform 1 | (+1) | | | |
| (Fragment) GN = ATP2A2 | | | | |
| Hepatocyte growth factor activator | HGFA_HUMAN | 71 | 0.03 | 0.01 |
| GN = HGFAC | | | | |
| Collagen alpha-1(VI) chain | A0A087X0S5_HUMAN | 108 | 0.03 | 0.01 |
| GN = COL6A1 | (+1) | | | |
| Plasminogen GN = PLG | PLMN_HUMAN | 91 | 0.03 | 0.01 |
| Myosin-6 GN = MYH6 | MYH6_HUMAN | 224 | 0.02 | 0.00 |
| Heparin cofactor 2 GN = SERPIND1 | HEP2_HUMAN | 57 | 0.03 | 0.00 |
| Carboxypeptidase N subunit 2 GN = CPN2 | CPN2_HUMAN | 61 | 0.03 | 0.01 |
| N-acetylmuramoyl-L-alanine amidase | PGRP2_HUMAN | 62 | 0.04 | 0.01 |
| GN = PGLYRP2 | | | | |
| Collagen alpha-3(VI) chain GN = | CO6A3_HUMAN (+1) | 344 | 0.02 | 0.01 |
| COL6A3 | | | | |
| Serpin peptidase inhibitor, clade A | A0A024R6I9_HUMAN | 49 | 0.03 | 0.01 |
| (Alpha-1 antiproteinase, antitrypsin), | (+2) | | | |
| member 4, isoform CRA_a | | | | |
| GN = SERPINA4 | | | | |
| Coagulation factor XIII B chain | F13B_HUMAN | 76 | 0.02 | 0.00 |
| GN = F13B | | | | |
| CDNA FLJ55769, highly similar to | B4DY96_HUMAN | 51 | 0.02 | 0.00 |
| Trifunctional enzyme subunit beta, | (+1) | | | |
| mitochondrial | | | | |
| Angiopoietin-like 6, isoform CRA_a | A0A024R7A9_HUMAN | 52 | 0.06 | 0.04 |
| GN = ANGPTL6 | (+1) | | | |
| Alpha-1B-glycoprotein GN = A1BG | A1BG_HUMAN (+1) | 54 | 0.02 | 0.02 |
| CDNA FLJ53494, highly similar to | B4DN90_HUMAN | 82 | 0.02 | 0.00 |
| Cartilage oligomeric matrix protein | (+2) | | | |
| Cholesteryl ester transfer protein plasma | A0A0S2Z3F6_HUMAN | 55 | 0.03 | 0.01 |
| isoform 1 (Fragment) GN = CETP | (+2) | | | |
| Coagulation factor XIII A chain | F13A_HUMAN | 83 | 0.02 | 0.01 |
| GN = F13A1 | | | | |
| Immunoglobulin delta heavy chain | IGD_HUMAN | 56 | 0.02 | 0.01 |
| Complement component C8 alpha chain | CO8A_HUMAN | 65 | 0.02 | 0.01 |
| GN = C8A | | | | |
| Inter-alpha-trypsin inhibitor heavy | ITIH3_HUMAN | 100 | 0.02 | 0.00 |
| chain H3 GN = ITIH3 | | | | |
| Protein HEG homolog 1 GN = HEG1 | HEG1_HUMAN | 147 | 0.01 | 0.01 |
| TNC variant protein (Fragment) | Q4LE33_HUMAN | 244 | 0.01 | 0.01 |
| GN = TNC variant protein | | | | |
| cDNA FLJ75881, highly similar to | A8K6Q8_HUMAN (+1) | 85 | 0.01 | 0.00 |
| Homo sapiens transferrin receptor (p90, | | | | |
| CD71) (TFRC), mRNA | | | | |
| Thrombospondin-1 GN = THBS1 | TSP1_HUMAN | 129 | 0.01 | 0.01 |
| Prolow-density lipoprotein receptor- | LRP1_HUMAN | 505 | 0.01 | 0.00 |
| related protein 1 GN = LRP1 | | | | |
| Reelin GN = RELN | J3KQ66_HUMAN (+1) | 388 | 0.00 | 0.00 |
| Laminin subunit beta-1 GN = LAMB1 | G3XAI2_HUMAN (+1) | 200 | 0.00 | 0.00 |
| Sushi, von Willebrand factor type A, | A0A0A0MSD0_HUMAN | 390 | 0.00 | 0.00 |
| EGF and pentraxin domain-containing | (+2) | | | |
| protein 1 GN = SVEP1 | | | | |
| Desmoplakin GN = DSP | DESP_HUMAN | 332 | 0.00 | 0.00 |
| Junction plakoglobin, isoform CRA_a | A0A024R1X8_HUMAN | 82 | 0.00 | 0.00 |
| GN = JUP | (+2) | | | |
| Glyceraldehyde-3-phosphate | G3P_HUMAN (+1) | 36 | 0.00 | 0.00 |
| dehydrogenase GN = GAPDH | | | | |
| Histone H4 GN = HIST1H4H | B2R4R0_HUMAN (+2) | 11 | 0.00 | 0.00 |
| Keratinocyte proline-rich protein | KPRP_HUMAN | 64 | 0.00 | 0.00 |
| GN = KPRP | | | | |
| Plakophilin 1 (Ectodermal dysplasia/skin | A0A024R952_HUMAN | 80 | 0.00 | 0.00 |
| fragility syndrome), isoform CRA_a | | | | |
| GN = PKP1 | | | | |
| Histone H2B GN = HIST1H2BJ | A0A024RCJ2_HUMAN | 14 | 0.00 | 0.00 |
| | (+4) | | | |
| Desmoglein-1 GN = DSG1 | DSG1_HUMAN | 114 | 0.00 | 0.00 |
| Galectin-7 GN = LGALS7 | LEG7_HUMAN | 15 | 0.00 | 0.00 |
| Histone H3 GN = H3F3B | B2R4P9_HUMAN (+10) | 15 | 0.00 | 0.00 |
| Calmodulin-like protein 3 GN = | CALL3_HUMAN | 17 | 0.00 | 0.00 |
| CALML3 | | | | |
| Annexin GN = ANXA2 | A0A024R5Z7_HUMAN | 39 | 0.00 | 0.00 |
| | (+2) | | | |
| ATP synthase subunit beta, | ATPB_HUMAN (+2) | 57 | 0.00 | 0.00 |
| mitochondrial GN = ATP5B | | | | |
| Liver histone H1e | A3R0T7_HUMAN (+6) | 22 | 0.00 | 0.00 |
| cDNA FLJ43122 fis, clone | B3KWI4_ HUMAN (+1) | 64 | 0.00 | 0.00 |
| CTONG3003737, highly similar to | | | | |
| Leucine-rich repeat-containing protein 15 | | | | |
| 14-3-3 protein sigma GN = SFN | 1433S_HUMAN | 28 | 0.00 | 0.00 |
| V-set and immunoglobulin domain- | VSIG8_HUMAN | 44 | 0.00 | 0.00 |
| containing protein 8 GN = VSIG8 | | | | |
| Heat shock cognate 71 kDa protein | E9PKE3_HUMAN (+2) | 69 | 0.00 | 0.00 |
| GN = HSPA8 | | | | |
| Heat shock protein beta-1 GN = HSPB1 | HSPB1_HUMAN (+1) | 23 | 0.00 | 0.00 |
| Histone H1.5 GN = HIST1H1B | H15_HUMAN | 23 | 0.00 | 0.00 |
| Peroxiredoxin-6 GN = PRDX6 | PRDX6_HUMAN (+1) | 25 | 0.00 | 0.00 |
| Galectin GN = hCG_22119 | A0A024R693_HUMAN | 26 | 0.00 | 0.00 |
| | (+6) | | | |
| 60S ribosomal protein L8 GN = RPL8 | RL8_HUMAN | 28 | 0.00 | 0.00 |
| Serpin B12 GN = SERPINB12 | SPB12_HUMAN | 46 | 0.00 | 0.00 |
| Elongation factor 1-alpha 1 GN = EEF1A1 | EF1A1_HUMAN (+8) | 50 | 0.00 | 0.00 |
| Tubulin alpha-1A chain GN = TUBA1A | TBA1A_HUMAN | 50 | 0.00 | 0.00 |
| Voltage-dependent anion channel 2, | A0A024QZN9_HUMAN | 34 | 0.00 | 0.00 |
| isoform CRA_a GN = VDAC2 | (+3) | | | |
| Cytosol aminopeptidase GN = LAP3 | AMPL_HUMAN (+1) | 56 | 0.00 | 0.00 |
| Protein-glutamine gamma- | TGM3_HUMAN | 77 | 0.00 | 0.00 |
| glutamyltransferase E GN = TGM3 | | | | |
| Desmoglein-4 GN = DSG4 | DSG4_HUMAN | 114 | 0.00 | 0.00 |
| Serine protease inhibitor Kazal-type 5 | ISK5_HUMAN | 121 | 0.00 | 0.00 |
| GN = SPINK5 | | | | |
| Band 3 anion transport protein | B3AT_HUMAN (+3) | 102 | 0.00 | 0.00 |
| GN = SLC4A1 | | | | |
| Hephaestin-like protein 1 GN = HEPHL1 | HPHL1_HUMAN | 132 | 0.00 | 0.00 |
| APOB protein GN = APOB | Q7Z7Q0_HUMAN | 92 | 0.52 | 0.89 |
| IGL@ protein | Q8N355_HUMAN | 25 | 0.45 | 0.78 |
| cDNA FLJ90170 fis, clone | Q8NCL6_HUMAN | 53 | 0.37 | 0.32 |
| MAMMA1000370, highly similar to | | | | |
| Ig alpha-1 chain C region | | | | |
| V2-17 protein (Fragment) GN = V2-17 | Q5NV90_HUMAN | 10 | 0.31 | 0.29 |
| Immunoglobulin heavy variable 3-53 | HV353_HUMAN (+1) | 13 | 0.30 | 0.52 |
| GN = IGHV3-53 | | | | |
| IGHV1-2 protein (Fragment) | A0A0F776Q1_HUMAN | 12 | 0.23 | 0.20 |
| GN = IGHV1-2 | | | | |
| Anti-(ED-B) scFV (Fragment) | A2KBC1_HUMAN | 25 | 0.22 | 0.37 |
| Anti-HER3 scFv (Fragment) | A2J422_HUMAN | 26 | 0.21 | 0.18 |
| Immunoglobulin kappa variable 4-1 | KV401_HUMAN | 13 | 0.21 | 0.36 |
| GN = IGKV4-1 | | | | |
| Rheumatoid factor RF-IP4 (Fragment) | A2J1M5_HUMAN | 10 | 0.20 | 0.35 |
| V5-6 protein (Fragment) GN = V5-6 | Q5NV92_HUMAN | 11 | 0.19 | 0.17 |
| Immunoglobulin kappa variable 3D-20 | KVD20_HUMAN | 13 | 0.18 | 0.32 |
| GN = IGKV3D-20 | | | | |
| NANUC-2 heavy chain (Fragment) | A2NKM7_HUMAN | 15 | 0.18 | 0.15 |
| Uncharacterized protein | Q6N092_HUMAN | 56 | 0.16 | 0.28 |
| DKFZp686K18196 (Fragment) | | | | |
| GN = DKFZp686K18196 | | | | |
| Uncharacterized protein | Q6N091_HUMAN | 54 | 0.14 | 0.12 |
| DKFZp686C02220 (Fragment) | | | | |
| GN = DKFZp686C02220 | | | | |
| Uncharacterized protein | Q6N094_HUMAN | 53 | 0.13 | 0.23 |
| DKFZp686O01196 | | | | |
| GN = DKFZp686O01196 | | | | |
| Uncharacterized protein | Q7Z379_HUMAN | 52 | 0.12 | 0.21 |
| DKFZp686K04218 (Fragment) | | | | |
| GN = DKFZp686K04218 | | | | |
| 10E8 light chain variable region | A0A193CHR5_HUMAN | 12 | 0.10 | 0.09 |
| (Fragment) | (+3) | | | |
| MS-D2 light chain variable region | A0A0X9USL5_HUMAN | 11 | 0.09 | 0.09 |
| (Fragment) | | | | |
| Rheumatoid factor RF-ET12 (Fragment) | A2J1N9_HUMAN | 11 | 0.09 | 0.16 |
| IgG H chain | S6BAM6_HUMAN | 34 | 0.09 | 0.15 |
| IgG H chain | S6BGE0_HUMAN | 32 | 0.08 | 0.15 |
| IBM-B2 light chain variable region | A0A0X9V9D6_HUMAN | 11 | 0.08 | 0.07 |
| (Fragment) | | | | |
| Myosin-reactive immunoglobulin kappa | Q9UL86_HUMAN | 12 | 0.08 | 0.13 |
| chain variable region (Fragment) | | | | |
| GCT-A2 heavy chain variable region | A0A125U0V4_HUMAN | 14 | 0.07 | 0.13 |
| (Fragment) | | | | |
| Immunoglobulin heavy variable 1-69 | HV169 HUMAN | 13 | 0.06 | 0.11 |
| GN = IGHV1-69 | | | | |
| Immunoglobulin heavy variable 3-35 | A0A0C4DH35_HUMAN | 13 | 0.05 | 0.09 |
| (non-functional) (Fragment) | | | | |
| GN = IGHV3-35 | | | | |
| Anti-folate binding protein (Fragment) | A2NYQ7_HUMAN (+2) | 11 | 0.05 | 0.08 |
| GN = HuC4lambda Vlambda | | | | |
| Cryocrystalglobulin CC2 lambda light | B1N7B9_HUMAN | 11 | 0.05 | 0.08 |
| chain variable region (Fragment) | | | | |
| Beta-2-glycoprotein 1 GN = APOH | APOH_HUMAN (+1) | 38 | 0.04 | 0.04 |
| Immunoglobulin kappa variable 1-13 | KV113_HUMAN (+1) | 13 | 0.04 | 0.07 |
| GN = IGKV1-13 | | | | |
| Heavy chain Fab (Fragment) | A2NYU7_HUMAN | 14 | 0.04 | 0.06 |
| IBM-A2 light chain variable region | A0A0X9T0I7_HUMAN | 12 | 0.03 | 0.05 |
| (Fragment) | | | | |
| Immunoglobulin kappa variable 1D-16 | KVD16_HUMAN | 13 | 0.03 | 0.05 |
| (Fragment) GN = IGKV1D-16 | | | | |
| IBM-B3 heavy chain variable region | A0A109PW50_HUMAN | 14 | 0.02 | 0.04 |
| (Fragment) | | | | |
| N90-VRC38.07 heavy chain variable | A0A1W6IYI6_HUMAN | 14 | 0.02 | 0.04 |
| region (Fragment) | | | | |
| Serum amyloid A protein GN = SAA1 | D3DQX7_HUMAN | 14 | 0.02 | 0.04 |
| Myosin, light polypeptide 3, alkali | A0A024R2Q5_HUMAN | 22 | 0.02 | 0.04 |
| ventricular, skeletal, slow, isoform | (+1) | | | |
| CRA_a GN = MYL3 | | | | |
| 40S ribosomal protein (Fragment) | A0A248RGE3_HUMAN | 17 | 0.02 | 0.04 |
| | (+34) | | | |
| Beta-globin GN = HBB | D9YZU5_HUMAN (+1) | 16 | 0.02 | 0.04 |
| Hemopexin GN = HPX | HEMO_HUMAN | 52 | 0.02 | 0.02 |
| Serpin peptidase inhibitor, clade A | A0A024R6N9_HUMAN | 46 | 0.02 | 0.02 |
| (Alpha-1 antiproteinase, antitrypsin), | (+1) | | | |
| member 5, isoform CRA_a | | | | |
| GN = SERPINA5 | | | | |
| CDNA FLJ55606, highly similar to | B7Z8Q2_HUMAN (+2) | 47 | 0.02 | 0.02 |
| Alpha-2-HS-glycoprotein | | | | |
| Collectin sub-family member 10 (C-type | A0A024R9J3_HUMAN | 31 | 0.02 | 0.03 |
| lectin), isoform CRA_a GN = COLEC10 | (+1) | | | |
| Sperm binding protein 1a | A0A1L1UHR1_HUMAN | 31 | 0.02 | 0.03 |
| | (+1) | | | |
| Apolipoprotein F GN = APOF | APOF_HUMAN (+1) | 35 | 0.01 | 0.03 |
| Uncharacterized protein | Q6MZL2_HUMAN | 35 | 0.01 | 0.03 |
| DKFZp686M0562 (Fragment) | | | | |
| GN = DKFZp686M0562 | | | | |
| Mannose-binding protein C GN = MBL2 | MBL2_HUMAN | 26 | 0.01 | 0.02 |
| HLA class I histocompatibility antigen, | A0A140T951_HUMAN | 27 | 0.01 | 0.02 |
| B-46 alpha chain (Fragment) | | | | |
| GN = HLA-B | | | | |
| Apolipoprotein A-V, isoform CRA_a | A0A0B4RUS7_HUMAN | 41 | 0.01 | 0.02 |
| GN = APOA5 | (+3) | | | |
| Phospholipid transfer protein, isoform | B3KUE5_HUMAN (+2) | 57 | 0.01 | 0.01 |
| CRA_c GN = PLTP | | | | |
| Gelsolin GN = GSN | A0A0A0MS51_HUMAN | 83 | 0.01 | 0.02 |
| | (+5) | | | |
| cDNA FLJ51409, highly similar to | B7Z832_HUMAN (+2) | 96 | 0.01 | 0.01 |
| Thrombospondin-4 | | | | |
| Stomatin, isoform CRA_a GN = STOM | A0A024R882_HUMAN | 32 | 0.01 | 0.02 |
| | (+3) | | | |
| Selenoprotein P (Fragment) GN = | A0A182DWH7_HUMAN | 35 | 0.01 | 0.02 |
| SELENOP | (+1) | | | |
| Guanine nucleotide binding protein (G | A0A024R056_HUMAN | 37 | 0.01 | 0.02 |
| protein), beta polypeptide 1, isoform | (+2) | | | |
| CRA_a GN = GNB1 | | | | |
| cDNA FLJ78207, highly similar to | A8K2T4_HUMAN (+2) | 93 | 0.01 | 0.02 |
| Human complement protein component | | | | |
| C7 mRNA | | | | |
| Protein disulfide-isomerase GN = P4HB | A0A024R8S5_HUMAN | 57 | 0.01 | 0.02 |
| | (+1) | | | |
| Oncoprotein-induced transcript 3 protein | OIT3_HUMAN | 60 | 0.01 | 0.01 |
| GN = OIT3 | | | | |
| Integrin alpha-Ilb GN = ITGA2B | ITA2B_HUMAN | 113 | 0.01 | 0.01 |
| Moesin GN = MSN | MOES_HUMAN (+1) | 68 | 0.01 | 0.01 |
| Afamin GN = AFM | AFAM_HUMAN | 69 | 0.01 | 0.01 |
| Insulin-like growth factor-binding | ALS_HUMAN (+2) | 66 | 0.01 | 0.01 |
| protein complex acid labile subunit | | | | |
| GN = IGFALS | | | | |
| Cartilage acidic protein 1 GN = CRTAC1 | A0A0C4DFP6_HUMAN | 70 | 0.00 | 0.01 |
| | (+1) | | | |
| cDNA FLJ78071, highly similar to | A8K8Z4_HUMAN (+1) | 105 | 0.00 | 0.01 |
| Human MHC class III complement | | | | |
| component C6 mRNA | | | | |
| cDNA FLJ77744, highly similar to | A8K9A9_HUMAN (+2) | 71 | 0.00 | 0.01 |
| Homo sapiens kallikrein B, plasma | | | | |
| (Fletcher factor) 1 (KLKB1), mRNA | | | | |
| Fermitin family homolog 3 GN = | URP2_HUMAN | 76 | 0.00 | 0.01 |
| FERMT3 | | | | |
| Integrin beta | B4DTY9_HUMAN (+3) | 84 | 0.00 | 0.01 |
| CFB | A0A1U9X7H2_HUMAN | 86 | 0.00 | 0.01 |
| | (+9) | | | |
| Collagen alpha-2(VI) chain GN = | CO6A2_HUMAN | 109 | 0.00 | 0.01 |
| COL6A2 | | | | |
| Laminin, gamma 1 (Formerly LAMB2), | A0A024R972_HUMAN | 174 | 0.00 | 0.00 |
| isoform CRA_a GN = LAMC1 | (+1) | | | |
| Titin GN = TTN | A0A0A0MTS7_HUMAN | 3994 | 0.00 | 0.00 |
| | (+3) | | | |

TABLE 3
Candidate corona protein biomarkers differentially expressed between healthy controls and early stage
ovarian carcinoma patients, as identified by proteomic analysis of the ex vivo NP coronas.
Full list of proteins identified by Progenesis QI for proteomics to be upregulated or downregulated in
early stage ovarian carcinoma patients in comparison with healthy controls classified from the highest
max fold-change to the lowest. Only proteins with p < 0.05 are shown.

| | | | |
|---|---|---|---|
| | | Anova | Max fold |
| Identified Protein (n = 202) | Accession Number | (p) | change |

UPREGULATED (n = 69)

| | | | |
|---|---|---|---|
| Vimentin GN = VIM | VIME_HUMAN | 8.57E−06 | 55.78 |
| Anion exchange protein GN = SLC4A1 | E2RVJ0_HUMAN | 3.00E−06 | 34.13 |
| Elongation factor 1-alpha (Fragment) | Q53GE9_HUMAN | 1.35E−04 | 27.26 |
| Signal recognition particle 54 kDa protein | G3V4F7_HUMAN | 2.18E−02 | 27.17 |
| GN = SRP54 | | | |
| Serum amyloid A-1 protein GN = SAA1 | SAA1_HUMAN | 2.87E−02 | 26.67 |
| Histone H2A GN = HIST1H2AC | A0A024R017_HUMAN | 2.23E−04 | 25.63 |
| EPB41 protein (Fragment) GN = EPB41 | Q1WWM3_HUMAN | 6.88E−05 | 20.80 |
| Spectrin beta chain GN = SPTB | B2RMN7_HUMAN | 3.68E−05 | 20.46 |
| Glycophorin GN = GPErik | Q14440_HUMAN | 1.58E−03 | 16.29 |
| Myosin-11 GN = MYH11 | MYH11_HUMAN | 8.45E−05 | 16.19 |
| Keratin, type II cytoskeletal 75 GN = KRT75 | K2C75_HUMAN | 9.25E−04 | 14.71 |
| cDNA FLJ50805, highly similar to Erythrocyte | B7Z4C3_HUMAN | 2.41E−04 | 14.45 |
| membrane protein band 4.2 | | | |
| Tubulin beta chain (Fragment) | Q6LC01_HUMAN | 1.22E−03 | 14.33 |
| Hemoglobin subunit beta GN = HBB | HBB_HUMAN | 2.42E−07 | 10.86 |
| Spectrin alpha chain, erythrocytic 1 GN = | SPTA1_HUMAN | 7.41E−05 | 10.48 |
| SPTA1 | | | |
| Mutant hemoglobin alpha 2 globin chain | A0A0K2BMD8_HUMAN | 5.91E−07 | 9.35 |
| GN = HBA2 | | | |
| Solute carrier family 2 (Facilitated glucose | Q0P512_HUMAN | 7.17E−04 | 9.13 |
| transporter), member 1 GN = SLC2A1 | | | |
| Tubulin beta-1 chain GN = TUBB1 | TBB1_HUMAN | 2.48E−02 | 8.65 |
| Histone H2B type 1-B GN = HIST1H2BB | H2B1B_HUMAN | 4.94E−04 | 8.57 |
| Tubulin alpha-1A chain GN = TUBA1A | TBA1A_HUMAN | 1.16E−03 | 7.55 |
| Epididymis luminal protein 4 GN = YWHAZ | D0PNI1_HUMAN | 1.40E−04 | 6.83 |
| L-lactate dehydrogenase B chain GN = LDHB | LDHB_HUMAN | 2.59E−04 | 6.75 |
| Aminopeptidase GN = ANPEP | A0A024RC61_HUMAN | 1.15E−02 | 6.54 |
| Histone H4 GN = HIST1H4H | B2R4R0_HUMAN | 1.23E−02 | 6.31 |
| Peptidyl-prolyl cis-trans isomerase A GN = | PPIA_HUMAN | 4.76E−04 | 6.00 |
| PPIA | | | |
| Actin, cytoplasmic 1 GN = ACTB | ACTB HUMAN | 9.69E−05 | 5.89 |
| Actin, aortic smooth muscle GN = ACTA2 | ACTA_HUMAN | 9.30E−05 | 5.83 |
| Pyruvate kinase PKLR GN = PKLR | KPYR_HUMAN | 2.26E−02 | 5.72 |
| cDNA FLJ44538 fis, clone UTERU3005159, | B3KX26_HUMAN | 4.48E−02 | 5.10 |
| highly similar to TNF receptor-associated | | | |
| factor 5 | | | |
| Coagulation factor XI GN = F11 | FA11_HUMAN | 2.11E−04 | 5.05 |
| Catalase GN = CAT | CATA_HUMAN | 1.96E−04 | 4.55 |
| ARP3 actin-related protein 3 homolog (Yeast), | A0A024RAI1_HUMAN | 6.33E−03 | 4.32 |
| isoform CRA_a GN = ACTR3 | | | |
| Integrin beta-3 GN = ITGB3 | ITB3_HUMAN | 2.80E−03 | 4.31 |
| cDNA FLJ38781 fis, clone LIVER2000216, | B3KTV0_HUMAN | 8.53E−04 | 4.11 |
| highly similar to HEAT SHOCK COGNATE | | | |
| 71 kDa PROTEIN | | | |
| Ankyrin-1 GN = ANK1 | ANK1_HUMAN | 4.65E−03 | 4.07 |
| Immunoglobulin heavy variable 3/OR16-12 | A0A075B7B8_HUMAN | 5.45E−04 | 4.07 |
| (non-functional) (Fragment) GN = | | | |
| IGHV3OR16-12 | | | |
| Glycoprotein Ib (Platelet), alpha polypeptide | A0A0C4DGZ8_HUMAN | 9.52E−05 | 3.86 |
| GN = GP1BA | | | |
| Tyrosine-protein kinase receptor GN = TPM3- | M1VPF4_HUMAN | 3.62E−02 | 3.76 |
| ROS1 | | | |
| RAP1B, member of RAS oncogene family, | A0A024RB87_HUMAN | 3.01E−03 | 3.60 |
| isoform CRA_a GN = RAP1B | | | |
| Multimerin-1 GN = MMRN1 | MMRN1_HUMAN | 7.47E−03 | 3.43 |
| Integrin alpha-Ilb GN = ITGA2B | ITA2B_HUMAN | 2.45E−02 | 3.19 |
| Apolipoprotein C-III GN = APOC3 | BOYIW2_HUMAN | 2.87E−03 | 3.11 |
| Ficolin-3 GN = FCN3 | FCN3_HUMAN | 2.20E−02 | 3.07 |
| Integrin beta-1 GN = ITGB1 | ITB1_HUMAN | 1.63E−03 | 3.01 |
| APOC4-APOC2 readthrough (NMD candidate) | K7ER74_HUMAN | 8.63E−04 | 3.01 |
| GN = APOC4-APOC2 | | | |
| Reelin GN = RELN | RELN_HUMAN | 1.38E−03 | 2.98 |
| Lipopolysaccharide-binding protein GN = LBP | LBP_HUMAN | 7.17E−03 | 2.96 |
| cDNA FLJ39539 fis, clone PUAEN2008228, | B3KUB8_HUMAN | 2.53E−02 | 2.95 |
| highly similar to Platelet glycoprotein 4 | | | |
| Glyceraldehyde-3-phosphate dehydrogenase | G3P_HUMAN | 1.42E−02 | 2.91 |
| GN = GAPDH | | | |
| Thrombospondin-1 GN = THBS1 | TSP1_HUMAN | 4.01E−02 | 2.73 |
| Thrombospondin 1, isoform CRA_a GN = | A0A024R9Q1_HUMAN | 4.01E−02 | 2.73 |
| THBS1 | | | |
| Moesin GN = MSN | MOES_HUMAN | 3.66E−03 | 2.63 |
| Sushi, von Willebrand factor type A, EGF | SVEP1_HUMAN | 2.63E−03 | 2.47 |
| and pentraxin domain-containing protein 1 | | | |
| GN = SVEP1 | | | |
| Apolipoprotein A-V, isoform CRA_a | A0A0B4RUS7_HUMAN | 1.11E−02 | 2.47 |
| GN = APOA5 | | | |
| Apolipoprotein C-I, isoform CRA_a | A0A024R0T8_HUMAN | 2.68E−02 | 2.47 |
| GN = APOC1 | | | |
| Filamin-A GN = FLNA | FLNA_HUMAN | 6.29E−03 | 2.46 |
| Hemicentin-1 GN = HMCN1 | HMCN1_HUMAN | 2.27E−03 | 2.46 |
| Apolipoprotein C-IV GN = APOC4 | APOC4_HUMAN | 4.16E−03 | 2.30 |
| cDNA FLJ60461, highly similar to | B4DF70_HUMAN | 3.77E−02 | 2.24 |
| Peroxiredoxin-2 (EC 1.11.1.15) | | | |
| Apolipoprotein M GN = APOM | APOM_HUMAN | 1.41E−02 | 2.03 |
| Peroxisomal bifunctional enzyme GN = | ECHP_HUMAN | 6.55E−04 | 1.89 |
| EHHADH | | | |
| 78 kDa glucose-regulated protein GN = | GRP78_HUMAN | 1.38E−02 | 1.84 |
| HSPA5 | | | |
| Apolipoprotein C-IV GN = APOC4 | A5YAK2_HUMAN | 1.11E−02 | 1.69 |
| Platelet-activating factor acetylhydrolase | A0A024RD39_HUMAN | 1.66E−03 | 1.62 |
| GN = PLA2G7 | | | |
| Apolipoprotein F GN = APOF | APOF_HUMAN | 3.12E−02 | 1.61 |
| Vascular endothelial growth factor receptor 3 | VGFR3_HUMAN | 3.03E−02 | 1.61 |
| GN = FLT4 | | | |
| Complement component 1, r subcomponent | Q53HT9_HUMAN | 4.21E−02 | 1.46 |
| variant (Fragment) | | | |
| cDNA FLJ75066, highly similar to Homo | A8K5J8_HUMAN | 4.21E−02 | 1.46 |
| sapiens complement component 1, r | | | |
| subcomponent (C1R), mRNA | | | |
| cAMP-responsive element modulator | H7C4X0_HUMAN | 4.17E−02 | 1.38 |
| (Fragment) GN = CREM | | | |

DOWNREGULATED (n = 133)

| | | | |
|---|---|---|---|
| Regucalcin GN = RGN | RGN_HUMAN | 6.04E−04 | 160.83 |
| Retinol-binding protein 4 GN = RBP4 | RET4_HUMAN | 1.51E−02 | 36.24 |
| AKAP350C | Q96KG3_HUMAN | 1.63E−02 | 28.97 |
| Beta-Ala-His dipeptidase GN = CNDP1 | CNDP1_HUMAN | 1.26E−13 | 16.58 |
| E3 ubiquitin-protein ligase TRIM56 | TRI56_HUMAN | 5.43E−05 | 10.90 |
| GN = TRIM56 | | | |
| Afamin GN = AFM | AFAM_HUMAN | 2.23E−02 | 5.17 |
| Uncharacterized protein GN = | Q6N095_HUMAN | 3.59E−02 | 4.87 |
| DKFZp686K03196 | | | |
| Transferrin variant (Fragment) | Q53H26_HUMAN | 5.67E−03 | 4.62 |
| cDNA, FLJ93914, highly similar to Homo | B2R8I2_HUMAN | 4.16E−06 | 4.37 |
| sapiens histidine-rich glycoprotein (HRG), | | | |
| mRNA | | | |
| Histidine-rich glycoprotein GN = HRG | HRG_HUMAN | 4.16E−06 | 4.37 |
| Vitamin D-binding protein GN = GC | D6RF35_HUMAN | 1.52E−02 | 4.31 |
| A disintegrin and metalloproteinase with | ATS13_HUMAN | 7.51E−03 | 4.00 |
| thrombospondin motifs 13 GN = ADAMTS13 | | | |
| Integrator complex subunit 4 GN = INTS4 | INT4_HUMAN | 2.42E−04 | 3.56 |
| Plasminogen GN = PLG | PLMN_HUMAN | 1.03E−03 | 3.55 |
| Phosphatidylinositol-glycan-specific | PHLD_HUMAN | 3.65E−08 | 3.41 |
| phospholipase D GN = GPLD1 | | | |
| UBX domain-containing protein 8 | A0A087WWA4_HUMAN | 1.27E−02 | 3.30 |
| (Fragment) GN = UBXN8 | | | |
| V5-6 protein (Fragment) GN = V5-6 | Q5NV92_HUMAN | 1.77E−02 | 3.27 |
| Serum albumin GN = ALB | ALBU_HUMAN | 1.48E−02 | 3.22 |
| Selenoprotein P (Fragment) GN = SELENOP | A0A182DWH7_HUMAN | 3.23E−08 | 3.08 |
| Serpin peptidase inhibitor, clade C | A0A024R944_HUMAN | 1.36E−03 | 3.07 |
| (Antithrombin), member 1, isoform CRA_a | | | |
| GN = SERPINC1 | | | |
| Transthyretin GN = TTR | A0A087WV45_HUMAN | 8.04E−07 | 3.05 |
| Immunoglobulin heavy variable 3-43 | HV343_HUMAN | 8.21E−04 | 2.94 |
| GN = IGHV3-43 | | | |
| cDNA FLJ53691, highly similar to | B4E1B2_HUMAN | 2.42E−02 | 2.84 |
| Serotransferrin | | | |
| Apolipoprotein A-IV GN = APOA4 | APOA4_HUMAN | 3.29E−04 | 2.78 |
| Immunoglobulin kappa variable 3D-20 | KVD20_HUMAN | 1.02E−02 | 2.74 |
| GN = IGKV3D-20 | | | |
| APOB protein (Fragment) GN = APOB | P78482_HUMAN | 6.33E−03 | 2.71 |
| Coagulation factor XII GN = F12 | A0A0R7FJH5_HUMAN | 6.12E−03 | 2.69 |
| Immunoglobulin kappa variable 6D-21 | KVD21_HUMAN | 5.36E−03 | 2.68 |
| GN = IGKV6D-21 | | | |
| Complement component C8 gamma | CO8G_HUMAN | 2.18E−03 | 2.64 |
| chain GN = C8G | | | |
| Vitronectin GN = VTN | VTNC_HUMAN | 3.92E−07 | 2.62 |
| Uncharacterized protein | Q6MZL2_HUMAN | 1.60E−04 | 2.55 |
| DKFZp686M0562 (Fragment) | | | |
| GN = DKFZp686M0562 | | | |
| Serpin peptidase inhibitor, clade A (Alpha- | A0A024R6N9_HUMAN | 1.52E−04 | 2.54 |
| 1 antiproteinase, antitrypsin), member 5, | | | |
| isoform CRA_a GN = SERPINA5 | | | |
| Mannan-binding lectin serine protease 1 | MASP1_HUMAN | 2.39E−04 | 2.44 |
| GN = MASP1 | | | |
| cDNA FLJ59854, highly similar to | B4DEU0_HUMAN | 8.17E−05 | 2.41 |
| Homo sapiens pitrilysin metallopeptidase | | | |
| 1 (PITRM1), mRNA | | | |
| N-acetylmuramoyl-L-alanine amidase | PGRP2_HUMAN | 5.74E−06 | 2.39 |
| GN = PGLYRP2 | | | |
| Rheumatoid factor light chain variable | A2NW98_HUMAN | 3.30E−03 | 2.37 |
| region (Fragment) | | | |
| Heavy chain Fab (Fragment) | A2NYV1_HUMAN | 4.24E−04 | 2.34 |
| IBM-A2 heavy chain variable region | A0A0X9T7Y9_HUMAN | 3.62E−02 | 2.33 |
| (Fragment) | | | |
| Anoctamin (Fragment) GN = ANO7 | H7C220_HUMAN | 4.51E−05 | 2.32 |
| Complement component C7 GN = C7 | CO7_HUMAN | 1.59E−03 | 2.32 |
| Immunoglobulin kappa variable 2D-2 | KVD29_HUMAN | 5.04E−04 | 2.31 |
| GN = IGKV2D-29 | | | |
| Immunoglobulin heavy constant | IGHG2_HUMAN | 1.59E−03 | 2.29 |
| gamma 2 GN = IGHG2 | | | |
| Uncharacterized protein | Q6N093_HUMAN | 1.59E−03 | 2.29 |
| DKFZp686I04196 (Fragment) | | | |
| GN = DKFZp686I04196 | | | |
| Uncharacterized protein | Q6MZU6_HUMAN | 1.59E−03 | 2.29 |
| DKFZp686C15213 | | | |
| GN = DKFZp686C15213 | | | |
| C4B (Fragment) GN = C4B | Q6U2L6_HUMAN | 6.10E−03 | 2.26 |
| Serum paraoxonase/arylesterase 1 | PON1_HUMAN | 3.90E−05 | 2.22 |
| GN = PON1 | | | |
| Alpha-1-antitrypsin GN = SERPINA1 | A0A024R6I7_HUMAN | 9.75E−03 | 2.20 |
| Alpha-1-antitrypsin GN = SERPINA1 | A1AT_HUMAN | 9.75E−03 | 2.20 |
| GCT-A8 light chain variable region | A0A109PS54_HUMAN | 4.26E−02 | 2.20 |
| (Fragment) | | | |
| Epididymis luminal protein 213 | V9HW34_HUMAN | 7.23E−04 | 2.20 |
| GN = HEL-213 | | | |
| Probable ATP-dependent RNA helicase | A0A0C4DG89_HUMAN | 8.49E−06 | 2.19 |
| DDX46 GN = DDX46 | | | |
| REV25-2 (Fragment) | A0N7J6_HUMAN | 4.42E−03 | 2.18 |
| IGK@ protein GN = IGK@ | Q6P5S8_HUMAN | 1.98E−03 | 2.15 |
| Immunoglobulin lambda variable 7-46 | LV746_HUMAN | 9.26E−03 | 2.13 |
| GN = IGLV7-46 | | | |
| Rho GTPase-activating protein 23 | A0A087WXU2_HUMAN | 7.21E−04 | 2.10 |
| (Fragment) GN = ARHGAP23 | | | |
| Angiotensinogen variant (Fragment) | Q53GY3_HUMAN | 3.78E−03 | 2.10 |
| Ceruloplasmin GN = CP | CERU_HUMAN | 1.36E−02 | 2.08 |
| Ig heavy chain variable region | A0A068LKQ2_HUMAN | 9.38E−04 | 2.07 |
| (Fragment) | | | |
| Immunoglobulin lambda variable 7-43 | LV743_HUMAN | 3.62E−02 | 2.04 |
| GN = IGLV7-43 | | | |
| VH6DJ protein (Fragment) GN = VH6DJ | A2N0T9_HUMAN | 1.17E−03 | 2.03 |
| Rheumatoid factor RF-IP24 (Fragment) | A2J1N4_HUMAN | 4.03E−02 | 2.03 |
| IGK@ protein GN = IGK@ | Q6PIL8_HUMAN | 1.03E−03 | 2.02 |
| Immunoglobulin lambda variable 10-54 | A0A1W2PQ80_HUMAN | 2.28E−02 | 2.01 |
| GN = IGLV10-54 | | | |
| cDNA FLJ90170 fis, clone | Q8NCL6_HUMAN | 1.64E−02 | 2.00 |
| MAMMA1000370, highly similar to | | | |
| Ig alpha-1 chain C region | | | |
| Myosin-reactive immunoglobulin heavy | Q9UL72_HUMAN | 4.99E−03 | 1.98 |
| chain variable region (Fragment) | | | |
| Ankyrin-3 GN = ANK3 | ANK3_HUMAN | 1.48E−04 | 1.98 |
| Plasma kallikrein (Fragment) GN = KLKB1 | H0YAC1_HUMAN | 9.99E−03 | 1.97 |
| Collectin sub-family member 10 (C-type | A0A024R9J3_HUMAN | 1.16E−03 | 1.97 |
| lectin), isoform CRA_a GN = COLEC10 | | | |
| Anti-H1N1 influenza HA kappa chain | G3GAU4_HUMAN | 3.84E−03 | 1.94 |
| variable region (Fragment) | | | |
| Myosin-reactive immunoglobulin light | Q9UL82_HUMAN | 5.36E−03 | 1.93 |
| chain variable region (Fragment) | | | |
| cDNA FLJ14473 fis, clone | Q96K68_HUMAN | 1.03E−02 | 1.93 |
| MAMMA1001080, highly similar to Homo | | | |
| sapiens SNC73 protein (SNC73) mRNA | | | |
| Flotillin-1 (Fragment) GN = FLOT1 | A0A140T9R1_HUMAN | 5.26E−03 | 1.92 |
| Immunoglobulin alpha-2 heavy chain | IGA2_HUMAN | 7.29E−03 | 1.92 |
| V1-3 protein (Fragment) GN = V1-3 | Q5NV84_HUMAN | 1.28E−02 | 1.91 |
| Complement component C8 beta chain | F5GY80_HUMAN | 4.87E−04 | 1.90 |
| GN = C8B | | | |
| cDNA FLJ78071, highly similar to Human | A8K8Z4_HUMAN | 1.20E−02 | 1.89 |
| MHC class III complement component | | | |
| C6 mRNA | | | |
| VH4 heavy chain variable region | O95973_HUMAN | 1.67E−02 | 1.88 |
| (Fragment) GN = IGM | | | |
| Clusterin GN = CLU | CLUS_HUMAN | 5.64E−05 | 1.86 |
| Complement component C6 GN = C6 | CO6_HUMAN | 5.93E−03 | 1.84 |
| Single-chain Fv (Fragment) GN = scFv | Q65ZC9_HUMAN | 2.40E−02 | 1.84 |
| Complement C3 GN = C3 | CO3_HUMAN | 5.33E−03 | 1.84 |
| AT-rich interactive domain-containing | ARI5B_HUMAN | 2.12E−02 | 1.84 |
| protein 5B GN = ARID5B | | | |
| Uncharacterized protein | Q7Z2U7_HUMAN | 1.68E−02 | 1.84 |
| Rheumatoid factor RF-ET11 (Fragment) | A2J1N8_HUMAN | 1.02E−02 | 1.83 |
| Hornerin GN = HRNR | HORN_HUMAN | 1.37E−02 | 1.83 |
| NADH-ubiquinone oxidoreductase chain 5 | A0A059RS62_HUMAN | 1.66E−03 | 1.82 |
| GN = ND5 | | | |
| Immunogobulin kappa, VJ region | A2NH53_HUMAN | 8.26E−03 | 1.81 |
| (Fragment) | | | |
| Complement component C8 alpha chain | CO8A_HUMAN | 1.44E−02 | 1.79 |
| GN = C8A | | | |
| CYP20A1 protein (Fragment) GN = CYP20A1 | Q567U3_HUMAN | 5.40E−03 | 1.77 |
| Collectin-11 GN = COLEC11 | COL11_HUMAN | 3.67E−02 | 1.76 |
| V5-2 protein (Fragment) GN = V5-2 | A2MYC8_HUMAN | 4.46E−02 | 1.76 |
| GCT-A3 heavy chain variable region | A0A0X9TD88_HUMAN | 1.19E−03 | 1.76 |
| (Fragment) | | | |
| Cryocrystalglobulin CC2 lambda light | B1N7B9_HUMAN | 7.22E−03 | 1.76 |
| chain variable region (Fragment) | | | |
| 5′-nucleotidase, ecto (CD73) GN = NT5E | Q6NZX3_HUMAN | 2.76E−02 | 1.75 |
| IGL@ protein GN = IGL@ | Q6PIK1_HUMAN | 8.65E−03 | 1.73 |
| Lectin galactoside-binding soluble 3 | A0A0S2Z3Y1_HUMAN | 1.67E−02 | 1.73 |
| binding protein isoform 1 (Fragment) | | | |
| GN = LGALS3BP | | | |
| Fibrous sheath-interacting protein 2 | FSIP2_HUMAN | 7.32E−03 | 1.72 |
| GN = FSIP2 | | | |
| Myosin-reactive immunoglobulin light | Q9UL70_HUMAN | 4.24E−02 | 1.72 |
| chain variable region (Fragment) | | | |
| Immunoglobulin lambda variable 3-27 | LV327_HUMAN | 3.15E−02 | 1.68 |
| GN = IGLV3-27 | | | |
| Fibrinogen alpha chain GN = FGA | FIBA_HUMAN | 3.93E−03 | 1.67 |
| Prolow-density lipoprotein receptor- | LRP1_HUMAN | 1.33E−04 | 1.66 |
| related protein 1 GN = LRP1 | | | |
| Immunglobulin heavy chain variable | Q0ZCI2_HUMAN | 6.68E−03 | 1.64 |
| region (Fragment) | | | |
| Pregnancy zone protein GN = PZP | PZP_HUMAN | 4.04E−02 | 1.63 |
| cDNA FLJ75416, highly similar to Homo | A8K5T0_HUMAN | 1.81E−02 | 1.62 |
| sapiens complement factor H (CFH), mRNA | | | |
| Heavy chain Fab (Fragment) | A2NYU8_HUMAN | 1.12E−02 | 1.62 |
| Apolipoprotein A-II GN = APOA2 | APOA2_HUMAN | 9.03E−04 | 1.61 |
| Alpha-2-antiplasmin GN = SERPINF2 | A2AP_HUMAN | 4.66E−03 | 1.60 |
| VH3 protein (Fragment) GN = VH3 | Q9Y509_HUMAN | 3.58E−02 | 1.60 |
| Coagulation factor XIII B chain GN=F13B | F13B_HUMAN | 2.28E−02 | 1.59 |
| Cryocrystalglobulin CC1 kappa light chain | B1N7B8_HUMAN | 4.54E−02 | 1.57 |
| variable region (Fragment) | | | |
| Complement C1s subcomponent GN = C1S | C1S_HUMAN | 1.37E−03 | 1.57 |
| Rheumatoid factor C6 light chain (Fragment) | A0N5G1_HUMAN | 4.31E−02 | 1.57 |
| GN = V-kappa-1 | | | |
| Hepatocyte growth factor activator | HGFA_HUMAN | 2.18E−02 | 1.57 |
| GN = HGFAC | | | |
| Fibrinogen gamma chain, isoform CRA_a | D3DP16_HUMAN | 3.23E−03 | 1.56 |
| GN = FGG | | | |
| Filaggrin-2 GN = FLG2 | FILA2_HUMAN | 1.29E−02 | 1.56 |
| Immunoglobulin heavy variable 1-18 | HV118_HUMAN | 3.76E−02 | 1.55 |
| GN = IGHV1-18 | | | |
| HRV Fab 026-VL (Fragment) | A2IPI5_HUMAN | 4.02E−02 | 1.55 |
| Protein Asterix (Fragment) GN = WDR83OS | M0R1D5_HUMAN | 4.21E−02 | 1.55 |
| IgG L chain | S6BAR0_HUMAN | 2.68E−02 | 1.54 |
| Anti-streptococcal/anti-myosin | Q96SB0_HUMAN | 2.37E−02 | 1.54 |
| immunoglobulin lambda light chain variable | | | |
| region (Fragment) | | | |
| Testicular tissue protein Li 70 | A0A140VJJ6_HUMAN | 1.10E−02 | 1.53 |
| Uncharacterized protein | Q6MZQ6_HUMAN | 2.19E−02 | 1.51 |
| DKFZp686G11190 | | | |
| GN = DKFZp686G11190 | | | |
| F5-20 (Fragment) GN = F5-20 | A0N7I9_HUMAN | 2.19E−02 | 1.51 |
| Uncharacterized protein | Q8NEJ1_HUMAN | 4.33E−02 | 1.50 |
| Uncharacterized protein | Q6DHW4_HUMAN | 4.88E−02 | 1.45 |
| NADH dehydrogenase [ubiquinone] 1 | H7C2R1_HUMAN | 1.89E−02 | 1.45 |
| alpha subcomplex subunit 3 (Fragment) | | | |
| GN = NDUFA3 | | | |
| Fibrinogen beta chain GN = FGB | FIBB_HUMAN | 2.68E−02 | 1.43 |
| IgG H chain | S6BGD4_HUMAN | 3.88E−02 | 1.43 |
| MS-F1 light chain variable region (Fragment) | A0A0X9V9B3_HUMAN | 3.55E−02 | 1.43 |
| Keratin, type II cytoskeletal 2 epidermal | K22E_HUMAN | 1.57E−02 | 1.39 |
| GN = KRT2 | | | |
| Protein S isoform 1 (Fragment) GN = PROS1 | A0A0S2Z4K3_HUMAN | 4.05E−02 | 1.30 |
| Serine/threonine-protein kinase LMTK3 | A0A0A0MQW5_HUMAN | 2.45E−02 | 1.27 |
| GN = LMTK3 | | | |
| Keratin, type I cytoskeletal 10 GN = KRT10 | K1C10_HUMAN | 2.66E−02 | 1.27 |

TABLE 4
Candidate corona protein biomarkers differentially expressed between
healthy controls and late stage ovarian carcinoma patients, as
identified by proteomic analysis of the ex vivo NP coronas.
Full list of proteins identified by Progenesis QI for
proteomics to be upregulated or downregulated in
late stage ovarian carcinoma patients in comparison with
healthy controls classified from the highest
max fold-change to the lowest. Only proteins with p < 0.05 are shown.

| | | | |
|---|---|---|---|
| | | | Max |
| | Accession | Anova | fold |
| Identified Protein (n = 265) | Number | (p) | change |

UPREGULATED (n = 73)

| | | | |
|---|---|---|---|
| Keratin-associated protein | KR131_ | 3.93E−02 | 89.73 |
| 13-1 GN = KRTAP13-1 | HUMAN | | |
| Keratin-associated protein 3-1 | KRA31_ | 5.05E−03 | 41.13 |
| GN = KRTAP3-1 | HUMAN | | |
| Keratin, type II cuticular | KRT86_ | 4.96E−02 | 24.26 |
| Hb6 GN = KRT86 | HUMAN | | |
| Keratin, type II cuticular | KRT81_ | 4.16E−02 | 18.62 |
| Hb1 GN = KRT81 | HUMAN | | |
| Tubulin beta chain (Fragment) | Q6LC01_ | 2.66E−03 | 18.38 |
| | HUMAN | | |
| Anion exchange protein | E2RVJ0_ | 1.36E−04 | 16.28 |
| GN = SLC4A1 | HUMAN | | |
| Coagulation factor XI GN = F11 | FA11_ | 1.00E−06 | 15.01 |
| | HUMAN | | |
| Tubulin beta-1 chain | TBB1_ | 7.14E−03 | 12.28 |
| GN = TUBB1 | HUMAN | | |
| Elongation factor | Q53GE9_ | 1.93E−03 | 11.65 |
| 1-alpha (Fragment) | HUMAN | | |
| Signal recognition particle | G3V4F7_ | 5.63E−03 | 11.07 |
| 54 kDa protein | HUMAN | | |
| GN = SRP54 | | | |
| Myosin-11 GN = MYH11 | MYH11_ | 7.07E−04 | 9.62 |
| | HUMAN | | |
| Zinc finger protein 621 | C9JZC2_ | 4.13E−04 | 9.26 |
| GN = ZNF621 | HUMAN | | |
| Spectrin beta chain GN = SPTB | B2RMN7_ | 5.53E−05 | 9.05 |
| | HUMAN | | |
| Tubulin alpha-1A chain | TBA1A_ | 7.54E−03 | 8.82 |
| GN = TUBA1A | HUMAN | | |
| Serum amyloid A-1 protein | SAA1_ | 1.85E−03 | 7.32 |
| GN = SAA1 | HUMAN | | |
| Integrin alpha-6 GN = ITGA6 | ITA6_ | 5.24E−03 | 7.31 |
| | HUMAN | | |
| L-lactate dehydrogenase | LDHB_ | 1.43E−03 | 6.78 |
| B chain GN = LDHB | HUMAN | | |
| Apolipoprotein C-III | B0YIW2_ | 9.73E−03 | 6.76 |
| GN = APOC3 | HUMAN | | |
| Actin, aortic smooth muscle | ACTA_ | 1.21E−03 | 6.54 |
| GN = ACTA2 | HUMAN | | |
| Apolipoprotein C-IV | APOC4_ | 1.73E−02 | 6.39 |
| GN = APOC4 | HUMAN | | |
| Ficolin-3 GN = FCN3 | FCN3_ | 5.77E−04 | 6.36 |
| | HUMAN | | |
| Actin, cytoplasmic 1 GN = ACTB | ACTB_ | 7.75E−04 | 6.12 |
| | HUMAN | | |
| APOC4-APOC2 readthrough | K7ER74_ | 3.51E−03 | 5.97 |
| (NMD candidate) | HUMAN | | |
| GN = APOC4-APOC2 | | | |
| Epididymis luminal protein | D0PNI1_ | 1.73E−02 | 5.89 |
| 4 GN = YWHAZ | HUMAN | | |
| Hemoglobin subunit beta | HBB_ | 1.89E−06 | 5.84 |
| GN = HBB | HUMAN | | |
| Multimerin-1 GN = MMRN1 | MMRN1_ | 1.78E−02 | 5.67 |
| | HUMAN | | |
| Zinc finger CCCH-type | C9J6P4_ | 4.19E−03 | 5.65 |
| antiviral protein 1 | HUMAN | | |
| GN = ZC3HAV1 | | | |
| Spectrin alpha chain, erythrocytic | SPTA1_ | 8.77E−04 | 5.54 |
| 1 GN = SPTA1 | HUMAN | | |
| Mutant hemoglobin alpha 2 | A0A0K2BMD8_ | 5.14E−06 | 5.53 |
| globin chain GN = HBA2 | HUMAN | | |
| cDNA FLJ50805, highly | B7Z4C3_ | 3.11E−03 | 5.34 |
| similar to Erythrocyte | HUMAN | | |
| membrane protein band 4.2 | | | |
| Solute carrier family 2 | Q0P512_ | 6.29E−03 | 5.13 |
| (Facilitated glucose | HUMAN | | |
| transporter), member 1 | | | |
| GN = SLC2A1 | | | |
| Aminopeptidase GN = ANPEP | A0A024RC61_ | 1.03E−02 | 5.07 |
| | HUMAN | | |
| Ras-related protein Rab-1A | RAB1A_ | 1.41E−02 | 4.88 |
| GN = RAB1A | HUMAN | | |
| cDNA FLJ77094, highly | A8K479_ | 2.68E−02 | 4.66 |
| similar to Homo sapiens | HUMAN | | |
| apolipoprotein B (including | | | |
| Ag(x) antigen) (APOB), | | | |
| mRNA (Fragment) | | | |
| RAP1B, member of RAS | A0A024RB87_ | 2.06E−02 | 4.63 |
| oncogene family, isoform | HUMAN | | |
| CRA_a GN = RAP1B | | | |
| Integrin beta-3 GN = ITGB3 | ITB3_ | 1.46E−02 | 4.52 |
| | HUMAN | | |
| Tenascin (Fragment) GN = TNC | H0YGZ3_ | 4.01E−03 | 4.21 |
| | HUMAN | | |
| Filamin-A GN = FLNA | FLNA_ | 6.69E−03 | 4.20 |
| | HUMAN | | |
| Catalase GN = CAT | CATA_ | 7.07E−05 | 4.06 |
| | HUMAN | | |
| cDNA FLJ38781 fis, clone | B3KTV0_ | 1.54E−03 | 4.06 |
| LIVER2000216, highly | HUMAN | | |
| similar to HEAT SHOCK | | | |
| COGNATE 71 kDa | | | |
| PROTEIN | | | |
| Sushi, von Willebrand factor | SVEP1_ | 1.03E−02 | 3.99 |
| type A, EGF and | HUMAN | | |
| pentraxin domain-containing | | | |
| protein 1 GN = SVEP1 | | | |
| Reelin GN = RELN | RELN_ | 4.72E−03 | 3.87 |
| | HUMAN | | |
| Integrin beta-1 GN = ITGB1 | ITB1_ | 1.78E−02 | 3.63 |
| | HUMAN | | |
| Apolipoprotein M GN = APOM | APOM_ | 1.66E−02 | 3.54 |
| | HUMAN | | |
| CREB/ATF bZIP transcription | H0YDC7_ | 5.44E−03 | 3.49 |
| factor (Fragment) | HUMAN | | |
| GN = CREBZF | | | |
| Integrin alpha-IIb GN = ITGA2B | ITA2B_ | 4.51E−02 | 3.20 |
| | HUMAN | | |
| Soluble scavenger receptor | SRCRL_ | 8.22E−03 | 3.17 |
| cysteine-rich domain- | HUMAN | | |
| containing protein SSC5D | | | |
| GN = SSC5D | | | |
| Glyceraldehyde-3-phosphate | G3P_ | 8.07E−03 | 3.12 |
| dehydrogenase | HUMAN | | |
| GN = GAPDH | | | |
| Glycoprotein Ib (Platelet), | A0A0C4DGZ8_ | 2.57E−03 | 3.09 |
| alpha polypeptide | HUMAN | | |
| GN = GP1BA | | | |
| Coagulation factor XI | H0Y596_ | 1.46E−03 | 3.05 |
| (Fragment) GN = F11 | HUMAN | | |
| BTB/POZ domain-containing | KCTD5_ | 2.37E−02 | 3.00 |
| protein KCTD5 | HUMAN | | |
| GN = KCTD5 | | | |
| Peroxisomal bifunctional enzyme | ECHP_ | 1.23E−03 | 2.97 |
| GN = EHHADH | HUMAN | | |
| FPGT-TNNI3K readthrough | V9GXZ4_ | 2.02E−02 | 2.71 |
| GN = FPGT-TNNI3K | HUMAN | | |
| RUN and FYVE domain- | H0YD93_ | 5.51E−03 | 2.66 |
| containing protein 2 | HUMAN | | |
| (Fragment) GN = RUFY2 | | | |
| AP complex subunit beta | A0A087X253_ | 2.96E−02 | 2.63 |
| GN = AP2B1 | HUMAN | | |
| Ankyrin-1 GN = ANK1 | ANK1_HUMAN | 7.14E−03 | 2.56 |
| Vinculin, isoform CRA_c | A0A024QZN4_ | 9.69E−03 | 2.56 |
| GN = VCL | HUMAN | | |
| Moesin GN = MSN | MOES_HUMAN | 2.87E−02 | 2.51 |
| Keratin, type II cytoskeletal | K2C8_HUMAN | 6.51E−04 | 2.44 |
| 8 GN = KRT8 | | | |
| Vascular endothelial growth | VGFR3_ | 4.16E−03 | 2.40 |
| factor receptor 3 | HUMAN | | |
| GN = FLT4 | | | |
| Proteoglycan 4, isoform | A0A024R930_ | 1.21E−02 | 2.32 |
| CRA_a GN = PRG4 | HUMAN | | |
| Neutral alpha-glucosidase | GANAB_ | 1.42E−02 | 2.28 |
| AB GN = GANAB | HUMAN | | |
| Fructose-bisphosphate aldolase | ALDOA_ | 1.73E−02 | 2.17 |
| A GN = ALDOA | HUMAN | | |
| Apolipoprotein F GN = APOF | APOF_HUMAN | 1.18E−02 | 2.16 |
| Platelet-activating factor | A0A024RD39_ | 2.78E−03 | 2.10 |
| acetylhydrolase | HUMAN | | |
| GN = PLA2G7 | | | |
| Bcl-2-associated transcription | A0A1W2PQ43_ | 4.53E−02 | 2.00 |
| factor 1 GN = BCLAF1 | HUMAN | | |
| Histone-lysine N- | KMT2D_ | 1.91E−02 | 1.98 |
| methyltransferase | HUMAN | | |
| 2D GN = KMT2D | | | |
| Fatty acid desaturase | A0A087WU67_ | 1.28E−02 | 1.92 |
| 6 GN = FADS6 | HUMAN | | |
| cAMP-responsive element | H7C4X0_ | 1.13E−02 | 1.89 |
| modulator (Fragment) | HUMAN | | |
| GN = CREM | | | |
| Tenascin-X GN = TNXB | A0A087X010_ | 1.09E−02 | 1.89 |
| | HUMAN | | |
| Zinc finger protein 687 | H0Y5I5_ | 2.03E−02 | 1.84 |
| (Fragment) GN = ZNF687 | HUMAN | | |
| Apolipoprotein B (Including | C0JYY2_ | 1.93E−02 | 1.82 |
| Ag(X) antigen) | HUMAN | | |
| GN = APOB | | | |
| Actinin alpha 4 isoform 3 | A0A0S2Z3C0_ | 3.85E−02 | 1.74 |
| (Fragment) GN = ACTN4 | HUMAN | | |

DOWNREGULATED (n = 192)

| | | | |
|---|---|---|---|
| Regucalcin GN = RGN | RGN_HUMAN | 7.89E−03 | 177.77 |
| Beta-Ala-His dipeptidase | CNDP1_ | 9.48E−09 | 18.28 |
| GN = CNDP1 | HUMAN | | |
| E3 ubiquitin-protein ligase | TRI56_HUMAN | 8.61E−03 | 7.94 |
| TRIM56 GN = TRIM56 | | | |
| Immunoglobulin kappa | KVD13_ | 1.56E−02 | 7.57 |
| variable 1D-13 | HUMAN | | |
| GN = IGKV1D-13 | | | |
| Immunoglobulin lambda variable | LV39_ | 1.32E−02 | 7.02 |
| 3-9 GN = IGLV3-9 | HUMAN | | |
| IgG H chain | S6AWF0_ | 1.72E−02 | 5.57 |
| | HUMAN | | |
| Phosphatidylinositol-glycan- | PHLD_ | 1.47E−08 | 5.44 |
| specific phospholipase | HUMAN | | |
| D GN = GPLD1 | | | |
| Immunoglobulin heavy variable | HVD82_ | 2.43E−02 | 5.38 |
| 4-38-2 GN = IGHV4-38-2 | HUMAN | | |
| Rheumatoid factor RF-IP24 | A2J1N4_ | 2.57E−03 | 5.27 |
| (Fragment) | HUMAN | | |
| Immunoglobulin kappa | KVD21_ | 2.30E−04 | 4.66 |
| variable 6D-21 | HUMAN | | |
| GN = IGKV6D-21 | | | |
| cDNA, FLJ93914, highly | B2R8I2_ | 1.30E−04 | 4.63 |
| similar to Homo sapiens | HUMAN | | |
| histidine-rich glycoprotein | | | |
| (HRG), mRNA | | | |
| Histidine-rich glycoprotein | HRG_HUMAN | 1.30E−04 | 4.63 |
| GN = HRG | | | |
| VH6DJ protein (Fragment) | A2N0U0_ | 1.73E−02 | 4.56 |
| GN = VH6DJ | HUMAN | | |
| Uncharacterized protein | Q6N095_ | 2.49E−02 | 4.26 |
| GN = DKFZp686K03196 | HUMAN | | |
| Myosin-reactive immunoglobulin | Q9UL90_ | 7.27E−03 | 4.14 |
| heavy chain | HUMAN | | |
| variable region (Fragment) | | | |
| C4B (Fragment) GN = C4B | Q6U2L6_ | 1.39E−03 | 3.84 |
| | HUMAN | | |
| IGK@ protein GN = IGK@ | Q6PIL8_ | 4.55E−05 | 3.68 |
| | HUMAN | | |
| GCT-A8 light chain variable | A0A109PS54_ | 2.90E−03 | 3.66 |
| region (Fragment) | HUMAN | | |
| Selenoprotein P (Fragment) | A0A182DWH7_ | 3.12E−06 | 3.63 |
| GN = SELENOP | HUMAN | | |
| MS-A6 heavy chain variable | A0A0X9USK2_ | 8.63E−03 | 3.63 |
| region (Fragment) | HUMAN | | |
| Precursor (AA −19 to 108) | A2NV54_ | 5.20E−03 | 3.40 |
| (Fragment) | HUMAN | | |
| Serpin peptidase inhibitor, | A0A024R6N9_ | 5.20E−05 | 3.40 |
| clade A (Alpha-1 | HUMAN | | |
| antiproteinase, antitrypsin), | | | |
| member 5, isoform | | | |
| CRA_a GN = SERPINA5 | | | |
| Heavy chain Fab (Fragment) | A2NYV1_ | 4.24E−04 | 3.24 |
| | HUMAN | | |
| Immunoglobulin lambda variable | LV327_ | 3.04E−04 | 3.18 |
| 3-27 GN = IGLV3-27 | HUMAN | | |
| Plasminogen GN = PLG | PLMN_HUMAN | 2.37E−02 | 3.17 |
| UBX domain-containing | A0A087WWA4_ | 2.12E−02 | 3.17 |
| protein 8 (Fragment) | HUMAN | | |
| GN = UBXN8 | | | |
| V5-6 protein (Fragment) | Q5NV92_ | 4.97E−02 | 3.17 |
| GN = V5-6 | HUMAN | | |
| Myosin-reactive immunoglobulin | Q9UL82_ | 8.13E−04 | 3.13 |
| light chain variable | HUMAN | | |
| region (Fragment) | | | |
| Serine/threonine-protein | A0A0A0MQW5_ | 2.56E−03 | 3.12 |
| kinase LMTK3 | HUMAN | | |
| GN = LMTK3 | | | |
| Immunoglobulin kappa | KVD20_ | 1.68E−02 | 3.06 |
| variable 3D-20 | HUMAN | | |
| GN = IGKV3D-20 | | | |
| IGK@ protein GN = IGK@ | Q6P5S8_ | 1.26E−04 | 3.05 |
| | HUMAN | | |
| Single-chain Fv (Fragment) | Q65ZC9_ | 3.26E−03 | 2.99 |
| GN = scFv | HUMAN | | |
| Immunoglobulin heavy variable | HV343_ | 2.39E−03 | 2.98 |
| 3-43 GN = IGHV3-43 | HUMAN | | |
| FLJ00382 protein (Fragment) | Q8NF20_ | 1.46E−02 | 2.98 |
| GN = FLJ00382 | HUMAN | | |
| Immunoglobulin delta heavy chain | IGD_HUMAN | 1.46E−02 | 2.98 |
| Myosin-reactive immunoglobulin | Q9UL72_ | 5.86E−03 | 2.96 |
| heavy chain | HUMAN | | |
| variable region (Fragment) | | | |
| Anti-H1N1 influenza HA kappa | G3GAU4_ | 8.46E−04 | 2.94 |
| chain variable | HUMAN | | |
| region (Fragment) | | | |
| Coagulation factor XII GN = F12 | A0A0R7FJH5_ | 1.15E−02 | 2.94 |
| | HUMAN | | |
| Transthyretin GN = TTR | A0A087WV45_ | 1.14E−04 | 2.92 |
| | HUMAN | | |
| Serpin peptidase inhibitor, | A0A024R944_ | 2.06E−02 | 2.92 |
| clade C (Antithrombin), | HUMAN | | |
| member 1, isoform CRA_a | | | |
| GN = SERPINC1 | | | |
| Angiotensinogen variant | Q53GY3_ | 3.13E−03 | 2.91 |
| (Fragment) | HUMAN | | |
| Heavy chain Fab (Fragment) | A2NYU9_ | 1.63E−02 | 2.90 |
| | HUMAN | | |
| Ig heavy chain variable | A0A068LRW6_ | 1.04E−02 | 2.89 |
| region (Fragment) | HUMAN | | |
| Vitronectin GN = VTN | VTNC_HUMAN | 1.44E−07 | 2.87 |
| A30 (Fragment) | A2MYE1_ | 5.80E−04 | 2.86 |
| | HUMAN | | |
| Plasma kallikrein (Fragment) | H0YAC1_ | 2.91E−03 | 2.84 |
| GN = KLKB1 | HUMAN | | |
| Polymeric immunoglobulin | PIGR_HUMAN | 7.22E−03 | 2.84 |
| receptor GN = PIGR | | | |
| Anoctamin (Fragment) | H7C220_ | 9.13E−05 | 2.84 |
| GN = ANO7 PE = 3 SV = 8 | HUMAN | | |
| Apolipoprotein A-IV | APOA4_ | 1.91E−03 | 2.82 |
| GN = APOA4 | HUMAN | | |
| IBM-B2 light chain variable | A0A0X9V9D6_ | 4.26E−02 | 2.81 |
| region (Fragment) | HUMAN | | |
| VH6DJ protein (Fragment) | A2N0T9_ | 5.30E−05 | 2.76 |
| GN = VH6DJ | HUMAN | | |
| Immunoglobulin heavy variable | HV349_ | 6.07E−04 | 2.76 |
| 3-49 GN = IGHV3-49 | HUMAN | | |
| Immunoglobulin lambda variable | LV746_ | 1.97E−03 | 2.73 |
| 7-46 GN = IGLV7-46 | HUMAN | | |
| V1-2 protein (Fragment) | A2MYD6_ | 7.99E−03 | 2.70 |
| GN = V1-2 | HUMAN | | |
| MS-A1 light chain variable | A0A109PSY4_ | 1.81E−02 | 2.69 |
| region (Fragment) | HUMAN | | |
| Ig heavy chain variable | A0A068LKQ2_ | 1.70E−04 | 2.68 |
| region (Fragment) | HUMAN | | |
| Uncharacterized protein | Q6MZL2_ | 1.03E−04 | 2.68 |
| DKFZp686M0562 | HUMAN | | |
| (Fragment) GN = | | | |
| DKFZp686M0562 | | | |
| N-acetylmuramoyl-L- | PGRP2_ | 5.74E−05 | 2.65 |
| alanine amidase | HUMAN | | |
| GN = PGLYRP2 | | | |
| CYP20A1 protein (Fragment) | Q567U3_ | 2.44E−03 | 2.65 |
| GN = CYP20A1 | HUMAN | | |
| Ankyrin-3 GN = ANK3 | ANK3_HUMAN | 1.48E−05 | 2.63 |
| Uncharacterized protein | Q8NEJ1_ | 3.94E−03 | 2.60 |
| | HUMAN | | |
| V5-2 protein (Fragment) | A2MYC8_ | 2.05E−02 | 2.59 |
| GN = V5-2 | HUMAN | | |
| Uncharacterized protein | Q6DHW4_ | 1.29E−03 | 2.57 |
| | HUMAN | | |
| N90-VRC38.04 heavy chain | A0A1W6IYI9_ | 5.52E−03 | 2.57 |
| variable region | HUMAN | | |
| (Fragment) | | | |
| Epididymis luminal protein | V9HW34_ | 3.38E−04 | 2.56 |
| 213 GN = HEL-213 | HUMAN | | |
| IGK@ protein GN = IGK@ | Q6PJF2_ | 5.55E−03 | 2.54 |
| | HUMAN | | |
| Serum paraoxonase/arylesterase | PON1_HUMAN | 1.52E−05 | 2.50 |
| 1 GN = PON1 | | | |
| MS-D1 light chain variable | A0A0X9TD47_ | 7.08E−03 | 2.49 |
| region (Fragment) | HUMAN | | |
| Fibrinogen alpha chain GN = FGA | FIBA_HUMAN | 9.80E−05 | 2.48 |
| K light chain variable | A2NXP9_ | 6.04E−03 | 2.46 |
| region (Fragment) | HUMAN | | |
| Immunoglobulin lambda variable | LV743_ | 3.50E−02 | 2.46 |
| 7-43 GN = IGLV7-43 | HUMAN | | |
| Immunoglobulin alpha-2 | IGA2_HUMAN | 8.22E−03 | 2.44 |
| heavy chain | | | |
| Myosin-reactive immunoglobulin | Q9UL86_ | 5.33E−03 | 2.42 |
| kappa chain | HUMAN | | |
| variable region (Fragment) | | | |
| Rho GTPase-activating protein | A0A087WXU2_ | 5.48E−04 | 2.42 |
| 23 (Fragment) | HUMAN | | |
| GN = ARHGAP23 | | | |
| Alpha-1-antitrypsin GN = | A1AT_HUMAN | 1.90E−02 | 2.41 |
| SERPINA1 | | | |
| Alpha-1-antitrypsin GN = | A0A024R6I7_ | 1.90E−02 | 2.41 |
| SERPINA1 | HUMAN | | |
| Burkitt's lymphoma | A0N2N3_ | 2.90E−02 | 2.40 |
| translocation t(2;8) encoding | HUMAN | | |
| kappa light chain,. Chromosome | | | |
| 8q+ break point | | | |
| (Fragment) | | | |
| cDNA FLJ59854, highly | B4DEU0_ | 1.04E−03 | 2.40 |
| similar to Homo sapiens | HUMAN | | |
| pitrilysin metallopeptidase | | | |
| 1 (PITRM1), mRNA | | | |
| Immunoglobulin heavy variable | HV70D_ | 1.75E−02 | 2.39 |
| 2-70D GN = IGHV2-70D | HUMAN | | |
| IgG H chain | S6BGD4_ | 5.12E−04 | 2.39 |
| | HUMAN | | |
| Coagulation factor XIII | F13B_HUMAN | 9.07E−04 | 2.39 |
| B chain GN = F13B | | | |
| Mannan-binding lectin | MASP1_ | 1.31E−03 | 2.37 |
| serine protease 1 | HUMAN | | |
| GN = MASP1 | | | |
| Fibrinogen gamma chain, | D3DP16_ | 4.09E−05 | 2.37 |
| isoform CRA_a GN = FGG | HUMAN | | |
| Immunoglobulin heavy variable | HV313_ | 1.54E−02 | 2.36 |
| 3-13 GN = IGHV3-13 | HUMAN | | |
| Alternative protein NIPA2 | L8E8V4_ | 2.45E−03 | 2.35 |
| GN = NIPA2 | HUMAN | | |
| MS-F1 light chain variable | A0A0X9V9B3_ | 5.83E−04 | 2.34 |
| region (Fragment) | HUMAN | | |
| Anti-streptococcal/anti-myosin | Q96SB0_ | 1.91E−03 | 2.34 |
| immunoglobulin | HUMAN | | |
| lambda light chain variable | | | |
| region (Fragment) | | | |
| IBM-A3 heavy chain | A0A0X9UWM4_ | 1.96E−02 | 2.33 |
| variable region (Fragment) | HUMAN | | |
| VH4 heavy chain variable | O95973_ | 7.83E−03 | 2.28 |
| region (Fragment) | HUMAN | | |
| GN = IGM | | | |
| Immunoglobulin heavy variable | HV205_HUMAN | 9.89E−03 | 2.27 |
| 2-5 GN = IGHV2-5 | | | |
| Uncharacterized protein | Q7Z2U7_ | 5.54E−03 | 2.27 |
| | HUMAN | | |
| REV25-2 (Fragment) | A0N7J6_ | 1.23E−02 | 2.26 |
| | HUMAN | | |
| V2-17 protein (Fragment) | Q5NV90_ | 4.27E−02 | 2.25 |
| GN = V2-17 | HUMAN | | |
| Intestinal mucin (Fragment) | O43419_ | 2.39E−03 | 2.24 |
| GN = MUC3 | HUMAN | | |
| Hepatocyte growth factor | HGFA_HUMAN | 6.86E−03 | 2.23 |
| activator GN = HGFAC | | | |
| Rheumatoid factor | A2J1N6_ | 2.55E−03 | 2.22 |
| RF-ET9 (Fragment) | HUMAN | | |
| Rheumatoid factor C6 | A0N5G1_ | 2.53E−03 | 2.21 |
| light chain (Fragment) | HUMAN | | |
| GN = V-kappa-1 | | | |
| Cryocrystalglobulin CC1 | B1N7B8_ | 5.36E−03 | 2.20 |
| kappa light chain variable | HUMAN | | |
| region (Fragment) | | | |
| Testicular tissue protein Li 70 | A0A140VJJ6_ | 3.70E−04 | 2.20 |
| | HUMAN | | |
| Uncharacterized | A0A0G2JRQ6_ | 1.00E−02 | 2.20 |
| protein (Fragment) | HUMAN | | |
| Complement component C8 | CO8G_ | 1.57E−02 | 2.17 |
| gamma chain | HUMAN | | |
| GN = C8G | | | |
| Cold agglutinin FS-2 | A2NB46_ | 3.57E−02 | 2.15 |
| L-chain (Fragment) | HUMAN | | |
| Immunogobulin kappa, | A2NH53_ | 1.23E−02 | 2.14 |
| VJ region (Fragment) | HUMAN | | |
| Rheumatoid factor | A2J1M8_ | 1.45E−02 | 2.14 |
| RF-IP12 (Fragment) | HUMAN | | |
| Rheumatoid factor light chain | A2NW98_ | 2.50E−02 | 2.13 |
| variable region | HUMAN | | |
| (Fragment) | | | |
| Anti-staphylococcal enterotoxin | A0A1L2BU33_ | 6.80E−03 | 2.13 |
| D heavy chain | HUMAN | | |
| variable region (Fragment) | | | |
| Cryocrystalglobulin CC2 lambda | B1N7B9_ | 1.20E−02 | 2.13 |
| light chain variable | HUMAN | | |
| region (Fragment) | | | |
| Immunoglobulin heavy variable | HV118_ | 3.98E−03 | 2.12 |
| 1-18 GN = IGHV1-18 | HUMAN | | |
| Complement component C7 | CO7_HUMAN | 1.65E−02 | 2.11 |
| GN = C7 | | | |
| N90-VRC38.09 heavy chain | A0A1W6IYJ1_ | 5.68E−03 | 2.10 |
| variable region | HUMAN | | |
| (Fragment) | | | |
| Immunoglobulin heavy variable | HV373_ | 3.00E−02 | 2.09 |
| 3-73 GN = IGHV3-73 | HUMAN | | |
| VH3 protein (Fragment) | Q9Y509_ | 9.22E−03 | 2.09 |
| GN = VH3 | HUMAN | | |
| Prolow-density lipoprotein | LRP1_ | 3.34E−04 | 2.08 |
| receptor-related protein | HUMAN | | |
| 1 GN = LRP1 | | | |
| Fibrous sheath-interacting | FSIP2_ | 5.33E−03 | 2.08 |
| protein 2 GN = FSIP2 | HUMAN | | |
| Immunoglobulin kappa variable | KV401_ | 6.46E−03 | 2.08 |
| 4-1 GN = IGKV4-1 | HUMAN | | |
| AT-rich interactive domain- | ARI5B_ | 1.29E−02 | 2.07 |
| containing protein 5B | HUMAN | | |
| GN = ARID5B | | | |
| Immunglobulin heavy chain | Q0ZCJ1_ | 2.81E−02 | 2.06 |
| variable region | HUMAN | | |
| (Fragment) | | | |
| MS-D4 heavy chain variable | A0A0X9UWK7_ | 1.23E−02 | 2.06 |
| region (Fragment) | HUMAN | | |
| Complement C3 GN = C3 | CO3_HUMAN | 6.46E−03 | 2.05 |
| Myosin-reactive immunoglobulin | Q9UL88_ | 1.87E−03 | 2.05 |
| heavy chain | HUMAN | | |
| variable region (Fragment) | | | |
| Uncharacterized protein | Q6MZQ6_ | 2.51E−03 | 2.05 |
| DKFZp686G11190 | HUMAN | | |
| GN = DKFZp686G11190 | | | |
| F5-20 (Fragment) GN = F5-20 | A0N7I9_ | 2.51E−03 | 2.05 |
| | HUMAN | | |
| Immunglobulin heavy chain | Q0ZCI2_ | 2.01E−03 | 2.05 |
| variable region | HUMAN | | |
| (Fragment) | | | |
| Immunoglobulin heavy variable | HV374_ | 1.61E−02 | 2.05 |
| 3-74 GN = IGHV3-74 | HUMAN | | |
| GCT-A3 heavy chain variable | A0A0X9TD88_ | 1.04E−04 | 2.04 |
| region (Fragment) | HUMAN | | |
| Fibrinogen beta chain GN = FGB | FIBB_HUMAN | 7.65E−04 | 2.03 |
| Immunoglobulin heavy | A0A0C4DH35_ | 1.76E−03 | 2.01 |
| variable 3-35 (non- | HUMAN | | |
| functional) (Fragment) | | | |
| GN = IGHV3-35 | | | |
| IBM-B2 heavy chain variable | A0A125QYY9_ | 9.94E−03 | 2.00 |
| region (Fragment) | HUMAN | | |
| Lectin galactoside-binding | A0A0S2Z3Y1_ | 1.29E−02 | 1.98 |
| soluble 3 binding protein | HUMAN | | |
| isoform 1 (Fragment) | | | |
| GN = LGALS3BP | | | |
| Collectin sub-family member | A0A024R9J3_ | 1.32E−02 | 1.97 |
| 10 (C-type lectin), | HUMAN | | |
| isoform CRA_a GN = COLEC10 | | | |
| Cortactin, isoform CRA_c | A0A024R5M3_ | 2.05E−02 | 1.97 |
| GN = CTTN | HUMAN | | |
| GCT-A5 heavy chain variable | A0A0X9T0H6_ | 2.06E−02 | 1.97 |
| region (Fragment) | HUMAN | | |
| Complement factor properdin | A0A0S2Z4I5_ | 1.80E−03 | 1.96 |
| isoform 1 (Fragment) | HUMAN | | |
| GN = CFP | | | |
| Immunoglobulin kappa light chain | IGK_HUMAN | 1.66E−03 | 1.96 |
| Cadherin EGF LAG | CELR2_ | 1.18E−03 | 1.95 |
| seven-pass G-type receptor 2 | HUMAN | | |
| GN = CELSR2 | | | |
| Alpha-2-antiplasmin | A2AP_HUMAN | 4.94E−04 | 1.95 |
| GN = SERPINF2 | | | |
| IBM-A1 heavy chain variable | A0A120HF66_ | 1.34E−02 | 1.94 |
| region (Fragment) | HUMAN | | |
| Immunoglobulin kappa variable | KV621_ | 2.73E−02 | 1.93 |
| 6-21 GN = IGKV6-21 | HUMAN | | |
| Probable ATP-dependent | A0A0C4DG89_ | 7.92E−04 | 1.93 |
| RNA helicase DDX46 | HUMAN | | |
| GN = DDX46 | | | |
| Immunglobulin heavy | Q0ZCH9_ | 3.29E−02 | 1.92 |
| chain variable region | HUMAN | | |
| (Fragment) | | | |
| Immunoglobulin heavy variable | HV372_ | 7.24E−03 | 1.92 |
| 3-72 GN = IGHV3-72 | HUMAN | | |
| MS-C1 heavy chain variable | A0A125U0U7_ | 2.10E−02 | 1.91 |
| region (Fragment) | HUMAN | | |
| Clusterin GN = CLU | CLUS_HUMAN | 6.49E−05 | 1.91 |
| Immunoglobulin J chain | IGJ_HUMAN | 1.89E−03 | 1.90 |
| GN = JCHAIN | | | |
| Complement component | CO8A_HUMAN | 1.88E−02 | 1.88 |
| C8 alpha chain GN = C8A | | | |
| Immunoglobulin kappa | KVD29_ | 1.26E−02 | 1.87 |
| variable 2D-29 | HUMAN | | |
| GN = IGKV2D-29 | | | |
| N90-VRC38.10 heavy | A0A1W6IYI8_ | 3.33E−02 | 1.86 |
| chain variable region | HUMAN | | |
| (Fragment) | | | |
| Microfibrillar protein | Q9NP29_ | 1.01E−02 | 1.86 |
| 2 (Fragment) | HUMAN | | |
| Myosin-reactive | Q9UL70_ | 4.49E−02 | 1.85 |
| immunoglobulin light | HUMAN | | |
| chain variable region (Fragment) | | | |
| NADH dehydrogenase | H7C2R1_ | 5.40E−03 | 1.85 |
| [ubiquinone] 1 alpha | HUMAN | | |
| subcomplex subunit 3 (Fragment) | | | |
| GN = NDUFA3 | | | |
| N90-VRC38.08 heavy chain | A0A1W6IYI5_ | 1.22E−02 | 1.83 |
| variable region | HUMAN | | |
| (Fragment) | | | |
| Immunoglobulin heavy variable | HV64D_ | 2.66E−02 | 1.81 |
| 3-64D GN = IGHV3-64D | HUMAN | | |
| Complement C1s | C1S_HUMAN | 1.31E−03 | 1.81 |
| subcomponent GN = C1S | | | |
| Calcium-binding mitochondrial | CMC1_ | 4.45E−02 | 1.79 |
| carrier protein | HUMAN | | |
| Aralar1 GN = SLC25A12 | | | |
| V1-3 protein (Fragment) | Q5NV84_ | 3.07E−02 | 1.76 |
| GN = V1-3 | HUMAN | | |
| GCT-A6 heavy chain | A0A109PVK5_ | 2.08E−02 | 1.76 |
| variable region (Fragment) | HUMAN | | |
| Microsomal triglyceride | MTP_ | 2.31E−02 | 1.75 |
| transfer protein large | HUMAN | | |
| subunit GN = MTTP | | | |
| cDNA FLJ75416, highly | A8K5T0_ | 7.49E−03 | 1.75 |
| similar to Homo sapiens | HUMAN | | |
| complement factor H | | | |
| (CFH), mRNA | | | |
| Protein Asterix (Fragment) | M0R1D5_ | 1.42E−02 | 1.75 |
| GN = WDR83OS | HUMAN | | |
| V1-13 protein (Fragment) | Q5NV69_ | 2.77E−02 | 1.74 |
| GN = V1-13 | HUMAN | | |
| Caveolae-associated protein 2 | CAVN2_ | 2.00E−03 | 1.74 |
| GN = CAVIN2 | HUMAN | | |
| Serine palmitoyltransferase, | A0A024R6H1_ | 7.42E−03 | 1.74 |
| long chain base | HUMAN | | |
| subunit 2, isoform CRA_a | | | |
| GN = SPTLC2 | | | |
| Fibulin-1 GN = FBLN1 | B1AHL2_ | 2.13E−02 | 1.73 |
| | HUMAN | | |
| Immunoglobulin heavy constant | IGHM_HUMAN | 6.00E−03 | 1.73 |
| mu GN = IGHM | | | |
| Complement component 1, q | A0A024RAB9_ | 5.54E−03 | 1.73 |
| subcomponent, B | HUMAN | | |
| chain, isoform CRA_a | | | |
| GN = C1QB | | | |
| Immunoglobulin lambda variable | LV319_ | 2.60E−02 | 1.73 |
| 3-19 GN = IGLV3-19 | HUMAN | | |
| NADH-ubiquinone | A0A059RS62_ | 1.18E−02 | 1.72 |
| oxidoreductase chain 5 | HUMAN | | |
| GN = ND5 | | | |
| Immunoglobulin kappa variable | KV228_ | 1.31E−02 | 1.69 |
| 2-28 GN = IGKV2-28 | HUMAN | | |
| Pregnancy zone protein GN = PZP | PZP_HUMAN | 4.26E−02 | 1.68 |
| Immunoglobulin heavy | Q9NPP6_ | 3.54E−02 | 1.67 |
| chain variant (Fragment) | HUMAN | | |
| Cold agglutinin FS-1 | A2NB45_ | 1.70E−02 | 1.66 |
| L-chain (Fragment) | HUMAN | | |
| V1-16 protein (Fragment) | Q5NV81_ | 4.45E−02 | 1.65 |
| GN = V1-16 | HUMAN | | |
| Myosin-reactive immunoglobulin | Q9UL73_ | 3.07E−02 | 1.64 |
| heavy chain | HUMAN | | |
| variable region (Fragment) | | | |
| Complement component 1, | A0A024RAA7_ | 7.94E−03 | 1.64 |
| q subcomponent, C | HUMAN | | |
| chain, isoform CRA_a | | | |
| GN = C1QC | | | |
| IGL@ protein GN = IGL@ | Q6PIK1_ | 1.43E−02 | 1.63 |
| | HUMAN | | |
| Protein S isoform 1 | A0A0S2Z4K3_ | 1.25E−03 | 1.62 |
| (Fragment) GN = PROS1 | HUMAN | | |
| Alpha-2-macroglobulin | A2MG_ | 4.77E−02 | 1.59 |
| GN = A2M | HUMAN | | |
| Cryocrystalglobulin CC1 | B1N7B6_ | 4.29E−03 | 1.58 |
| heavy chain variable | HUMAN | | |
| region (Fragment) | | | |
| Complement component | F5GY80_ | 2.03E−02 | 1.58 |
| C8 beta chain GN = C8B | HUMAN | | |
| Ubiquitinyl hydrolase 1 | A0A024R8A9_ | 3.55E−02 | 1.57 |
| GN = USP20 | HUMAN | | |
| Coagulation factor XIII A | F13A_ | 3.58E−02 | 1.56 |
| chain GN = F13A1 | HUMAN | | |
| Full-length cDNA clone | Q86TT1_ | 1.95E−02 | 1.55 |
| CS0DD006YL02 of | HUMAN | | |
| Neuroblastoma of | | | |
| Homo sapiens (human) | | | |
| CD5 antigen-like GN = CD5L | CD5L_ | 1.23E−02 | 1.54 |
| | HUMAN | | |
| C4b-binding protein beta chain | C4BPB_ | 4.39E−04 | 1.51 |
| GN = C4BPB | HUMAN | | |
| Complement component C6 | CO6_ | 4.34E−02 | 1.51 |
| GN = C6 | HUMAN | | |
| IgG L chain | S6BAR0_ | 2.75E−02 | 1.49 |
| | HUMAN | | |
| Apolipoprotein A-I, isoform | A0A024R3E3_ | 3.80E−02 | 1.49 |
| CRA_a GN = APOA1 | HUMAN | | |
| cDNA FLJ60320, highly | B4DPS0_ | 3.93E−02 | 1.48 |
| similar to Tyrosine-protein | HUMAN | | |
| phosphatase non-receptor | | | |
| type6 (EC 3.1.3.48) | C4BPA_ | | |
| C4b-binding protein alpha | HUMAN | 1.30E−03 | 1.46 |
| chain GN = C4BPA | | | |
| cDNA FLJ51597, highly | B4E1D8_ | 1.30E−03 | 1.46 |
| similar to C4b-binding | HUMAN | | |
| protein alpha chain | | | |
| Integrator complex subunit 4 | INT4_ | 3.39E−02 | 1.41 |
| GN = INTS4 | HUMAN | | |
| Complement component 1, | A0A024RAG6_ | 1.72E−02 | 1.37 |
| q subcomponent, A | HUMAN | | |
| chain, isoform CRA_a | | | |
| GN = C1QA | | | |

TABLE 5
Candidate corona protein biomarkers differentially expressed between
early and late stage ovarian carcinoma patients, as
identified by proteomic analysis of the ex vivo NP coronas.
Full list of proteins identified by Progenesis QI for
proteomics to be upregulated or downregulated in
late stage ovarian carcinoma patients in comparison
with early stage ovarian carcinoma patients
classified from the highest max fold-change
to the lowest. Only proteins with p < 0.05 are shown.

| | | | |
|---|---|---|---|
| | | | Max |
| | Accession | Anova | fold |
| Identified Protein (n = 50) | Number | (p) | change |

UPREGULATED (n = 25)

| | | | |
|---|---|---|---|
| Keratin-associated protein 9-2 | A0A140TA58_ | 4.72E−02 | Infinity |
| GN = KRTAP9-2 | HUMAN | | |
| Keratin associated protein | Q3LI55_ | 4.47E−02 | 1596.12 |
| GN = KRTAP11-1 | HUMAN | | |
| Keratin-associated protein 13-1 | KR131_ | 4.08E−02 | 55.16 |
| GN = KRTAP13-1 | HUMAN | | |
| Keratin-associated protein 3-1 | KRA31_ | 3.29E−03 | 23.28 |
| GN = KRTAP3-1 | HUMAN | | |
| Keratin, type II cuticular Hb6 | KRT86_ | 4.65E−03 | 22.92 |
| GN = KRT86 | HUMAN | | |
| Keratin, type II cuticular Hb1 | KRT81 | 6.11E−03 | 16.36 |
| GN = KRT81 | HUMAN | | |
| HLA class I histocompatibility | 1B57_ | 1.92E−02 | 11.43 |
| antigen, B-57 | | | |
| alpha chain GN = HLA-B | HUMAN | | |
| Flotillin-1 (Fragment) | A0A140T9R1_ | 2.55E−04 | 10.74 |
| GN = FLOT1 | HUMAN | | |
| cDNA FLJ43122 fis, clone | B3KWI4_ | 3.02E−02 | 4.09 |
| CTONG3003737, | HUMAN | | |
| highly similar to Leucine-rich | | | |
| repeat-containing | | | |
| protein 15 | | | |
| Zinc finger CCCH-type | C9J6P4_ | 5.57E−03 | 3.95 |
| antiviral protein 1 | HUMAN | | |
| GN = ZC3HAV1 | | | |
| Zinc finger protein 621 | C9JZC2_ | 2.11E−02 | 3.21 |
| GN = ZNF621 | HUMAN | | |
| Coagulation factor XI GN = F11 | FA11_ | 5.30E−03 | 2.97 |
| | HUMAN | | |
| Coagulation factor | H0Y596_ | 3.42E−03 | 2.21 |
| XI (Fragment) | HUMAN | | |
| GN = F11 | | | |
| Vinculin, isoform CRA_c | A0A024QZN4_ | 6.53E−03 | 2.18 |
| GN = VCL | HUMAN | | |
| CREB/ATF bZIP | H0YDC7_ | 1.55E−02 | 2.09 |
| transcription factor | HUMAN | | |
| (Fragment) GN = CREBZF | | | |
| Soluble scavenger receptor | SRCRL_ | 2.54E−02 | 2.07 |
| cysteine-rich | HUMAN | | |
| domain-containing protein | | | |
| SSC5D GN = SSC5D | | | |
| FPGT-TNNI3K readthrough | V9GXZ4_ | 2.59E−02 | 1.92 |
| GN = FPGT-TNNI3K | HUMAN | | |
| Fructose-bisphosphate aldolase | ALDOA_ | 1.65E−02 | 1.90 |
| A GN = ALDOA | HUMAN | | |
| RUN and FYVE | H0YD93_ | 2.09E−02 | 1.87 |
| domain-containing protein 2 | HUMAN | | |
| (Fragment) GN = RUFY2 | | | |
| Pescadillo homolog GN = PES1 | PESC_ | 3.07E−02 | 1.76 |
| | HUMAN | | |
| Proteoglycan 4, isoform | A0A024R930_ | 4.16E−02 | 1.76 |
| CRA_a GN = PRG4 | HUMAN | | |
| Neutral alpha-glucosidase AB | GANAB_ | 4.74E−02 | 1.75 |
| GN = GANAB | HUMAN | | |
| PH-interacting protein | PHIP_ | 2.80E−02 | 1.70 |
| GN = PHIP | HUMAN | | |
| Histone-lysine | KMT2D_ | 2.63E−02 | 1.69 |
| N-methyltransferase 2D | HUMAN | | |
| GN = KMT2D | | | |
| Zinc finger protein 687 (Fragment) | H0Y5I5_ | 2.19E−02 | 1.63 |
| GN = ZNF687 | HUMAN | | |

DOWNREGULATED (n = 25)

| | | | |
|---|---|---|---|
| Histone H2A GN = | A0A024R017_ | 1.89E−02 | 10.35 |
| HIST1H2AC | HUMAN | | |
| POTE ankyrin domain | POTEJ_ | 1.36E−02 | 7.42 |
| family member J | HUMAN | | |
| GN = POTEJ | | | |
| Histone H2B type 1-B | H2B1B_ | 2.19E−02 | 5.28 |
| GN = HIST1H2BB | HUMAN | | |
| Immunoglobulin heavy | HV226_ | 2.67E−02 | 4.58 |
| variable 2-26 | HUMAN | | |
| GN = IGHV2-26 | | | |
| Immunoglobulin heavy | A0A075B7B8_ | 5.68E−03 | 2.72 |
| variable 3/OR16-12 | HUMAN | | |
| (non-functional) (Fragment) | | | |
| GN = IGHV3OR16-12 | | | |
| Cortactin, isoform CRA_c | A0A024R5M3_ | 4.85E−02 | 2.50 |
| GN = CTTN | HUMAN | | |
| MS-D1 light chain variable | A0A0X9TD47_ | 5.37E−03 | 2.30 |
| region (Fragment) | HUMAN | | |
| Heavy chain Fab (Fragment) | A2NYU9_ | 3.48E−02 | 2.13 |
| | HUMAN | | |
| Anti-staphylococcal enterotoxin | A0A1L2BU33_ | 1.72E−02 | 2.13 |
| D heavy chain | HUMAN | | |
| variable region (Fragment) | | | |
| Polymeric immunoglobulin | PIGR_ | 2.14E−04 | 2.04 |
| receptor GN = PIGR | HUMAN | | |
| Immunoglobulin | HV102_ | 2.26E−02 | 2.03 |
| heavy variable 1-2 | HUMAN | | |
| GN = IGHV1-2 | | | |
| Myosin-reactive | Q9UL88_ | 2.14E−02 | 1.99 |
| immunoglobulin | HUMAN | | |
| heavy chain | | | |
| variable region (Fragment) | | | |
| Myosin-reactive | Q9UL86_ | 1.80E−02 | 1.99 |
| immunoglobulin | HUMAN | | |
| kappa chain | | | |
| variable region (Fragment) | | | |
| Immunoglobulin heavy variable | HV349_ | 1.57E−02 | 1.96 |
| 3-49 | HUMAN | | |
| GN = IGHV3-49 | | | |
| N90-VRC38.08 heavy | A0A1W6IYI5_ | 1.53E−02 | 1.85 |
| chain variable region | HUMAN | | |
| (Fragment) | | | |
| IGK@ protein GN = IGK@ | Q6PIL8_ | 4.05E−02 | 1.82 |
| | HUMAN | | |
| Immunoglobulin heavy variable | A0A0C4DH35_ | 3.52E−02 | 1.79 |
| 3-35 (non- | HUMAN | | |
| functional) (Fragment) | | | |
| GN = IGHV3-35 | | | |
| Uncharacterized protein | Q8NEJ1_ | 3.62E−02 | 1.74 |
| | HUMAN | | |
| Alpha-2-macroglobulin | A2MG_ | 2.19E−02 | 1.68 |
| GN = A2M | HUMAN | | |
| IgG H chain | S6BGD4_ | 2.55E−02 | 1.67 |
| | HUMAN | | |
| MS-F1 light chain variable | A0A0X9V9B3_ | 3.92E−02 | 1.64 |
| region (Fragment) | HUMAN | | |
| Phosphatidylinositol- | PHLD_ | 2.54E−02 | 1.59 |
| glycan-specific | HUMAN | | |
| phospholipase D GN = GPLD1 | | | |
| Fibrinogen gamma chain, | D3DP16_ | 2.62E−02 | 1.52 |
| isoform CRA_a | HUMAN | | |
| GN = FGG | | | |
| Cryocrystalglobulin CC1 | B1N7B6_ | 1.29E−02 | 1.52 |
| heavy chain variable | HUMAN | | |
| region (Fragment) | | | |
| Angiotensinogen | Q53GY3_ | 1.94E−02 | 1.39 |
| variant (Fragment) | HUMAN | | |

TABLE 6
Mass Spectrometry-based lipidomic analysis.
List of all complex lipids identified in healthy human plasma and
onto the surface of HSPC:CHOL liposomes,
as these were found by LC-MS/MS. All samples were run in
both positive and negative mode. Raw abundance
values are shown below for all complex lipids identified.

| | | | | | | | Corona- |
|---|---|---|---|---|---|---|---|
| | | | | | Bare | Corona- | coated |
| | | RT | lipid | Bare | NPs + | coated | NPs + |
| Compound | m/z | (min) | cocktail | NPs | STD | NPs | STD |
| CE 16:0 | 647.57 | 1.19 | 1 | 2 | 2 | 15 | 18 |
| CE 18:0 | 675.60 | 1.20 | 0 | 0 | 0 | 23 | 21 |
| CE 18:2 | 671.58 | 1.17 | 0 | 0 | 1 | 593 | 584 |
| CE 18:3 | 669.56 | 1.19 | 0 | 0 | 1 | 17 | 13 |
| CE 20:4 | 695.58 | 1.21 | 6 | 1 | 3 | 328 | 291 |
| CE 20:5 | 693.56 | 1.23 | 0 | 0 | 0 | 7 | 9 |
| CE 22:5 | 721.58 | 1.37 | 3 | 944 | 340 | 595 | 752 |
| CE 22:6 | 719.58 | 1.25 | 0 | 9 | 4 | 88 | 106 |
| CE std | 635.50 | 1.38 | 14 | 199 | 92 | 116 | 92 |
| DG 31:0 | 572.44 | 1.79 | 0 | 109 | 206 | 124 | 89 |
| DG 33:0 | 600.37 | 1.46 | 0 | 4 | 6 | 2 | 2 |
| DG 34:0 | 614.49 | 1.75 | 2 | 5 | 7 | 7 | 8 |
| DG 34:4 | 606.62 | 1.72 | 0 | 0 | 1 | 8 | 17 |
| DG 34:5 | 604.54 | 1.43 | 153 | 0 | 101 | 121 | 257 |
| DG 35:0 | 628.51 | 1.54 | 0 | 39 | 46 | 26 | 32 |
| DG 36:2 | 638.57 | 1.68 | 0 | 13 | 26 | 13 | 0 |
| DG 36:3 | 636.56 | 1.50 | 1 | 1 | 8 | 16 | 33 |
| DG 36:4 | 634.54 | 1.52 | 0 | 0 | 0 | 5 | 10 |
| DG 38:1 | 668.53 | 1.54 | 1 | 0 | 0 | 13 | 26 |
| DG 38:5 | 660.56 | 1.68 | 0 | 54 | 54 | 54 | 8 |
| DG 39:4 | 676.53 | 1.69 | 0 | 14 | 20 | 15 | 2 |
| DG 41:5 | 702.50 | 1.94 | 3 | 287 | 258 | 293 | 252 |
| DG 44:0 | 754.45 | 1.42 | 3 | 0 | 3 | 0 | 1 |
| DG 44:4 | 746.55 | 1.48 | 13 | 0 | 9 | 0 | 7 |
| DG std | 643.60 | 1.42 | 1408 | 0 | 872 | 176 | 781 |
| STD without H2O | 608.57 | 1.42 | 141589 | 129 | 106962 | 83 | 86217 |
| LPC 14:0 | 468.31 | 4.45 | 1 | 1 | 0 | 15 | 30 |
| LPC 15:0 | 482.35 | 4.40 | 2 | 1 | 1 | 242 | 419 |
| LPC 16:0 | 496.34 | 4.44 | 2 | 1923 | 2362 | 18924 | 31271 |
| LPC 16:1 | 494.32 | 4.47 | 1 | 0 | 0 | 6 | 29 |
| LPC 17:0 | 510.36 | 4.42 | 2 | 27 | 24 | 476 | 772 |
| LPC 18:0 | 524.37 | 4.43 | 0 | 23846 | 29397 | 28888 | 44533 |
| LPC 18:2 | 520.34 | 4.50 | 0 | 1 | 3 | 391 | 1116 |
| LPC 18:3 | 518.32 | 4.44 | 0 | 220 | 341 | 2210 | 3613 |
| LPC 19:0 | 538.39 | 4.41 | 0 | 2 | 3 | 35 | 59 |
| LPC 20:0 | 552.40 | 4.41 | 0 | 49 | 60 | 58 | 91 |
| LPC 20:1 | 550.39 | 4.45 | 0 | 0 | 0 | 18 | 52 |
| LPC 20:2 | 548.51 | 4.42 | 3 | 0 | 17 | 0 | 5 |
| LPC 20:3 | 546.35 | 4.43 | 3 | 3033 | 4423 | 3698 | 5602 |
| LPC 20:5 | 542.32 | 4.50 | 0 | 0 | 0 | 27 | 152 |
| LPC 22:3 | 574.35 | 4.73 | 0 | 7 | 3 | 0 | 1 |
| LPC 22:4 | 572.37 | 4.54 | 0 | 1 | 0 | 1 | 8 |
| LPC 22:6 | 568.33 | 4.42 | 3 | 24 | 30 | 21 | 22 |
| LPC std | 640.52 | 4.38 | 3395 | 1 | 3041 | 1 | 3066 |
| PC 28:0 | 678.51 | 3.82 | 2 | 2 | 2 | 4 | 5 |
| PC 30:1 | 704.53 | 3.86 | 0 | 0 | 2 | 4 | 5 |
| PC 30:0 | 706.54 | 3.82 | 2 | 1 | 1 | 150 | 187 |
| PC 32:1p/PC 32:2e | 716.56 | 3.86 | 1 | 0 | 0 | 1 | 5 |
| PC 32:0p/PC 32:1e | 718.58 | 3.83 | 2 | 0 | 1 | 122 | 180 |
| PC 32:0e | 720.59 | 3.78 | 1 | 4 | 4 | 377 | 599 |
| PC 32:3 | 728.52 | 3.82 | 0 | 0 | 0 | 0 | 0 |
| PC 32:2 | 730.54 | 3.89 | 2 | 0 | 2 | 297 | 401 |
| PC 32:1 | 732.56 | 3.85 | 1 | 1 | 2 | 421 | 537 |
| PC 32:0 | 734.57 | 3.83 | 2 | 1347 | 1354 | 2131 | 2441 |
| PC 34:3p/PC 34:4e | 740.56 | 3.83 | 0 | 0 | 0 | 1 | 2 |
| PC 33:3/34:2p/34:3e | 742.58 | 3.90 | 0 | 1 | 0 | 879 | 1183 |
| PC 33:2/34:1p/34:2e | 744.59 | 3.83 | 3 | 8 | 10 | 1481 | 2019 |
| PC 33:0/34:0e | 748.59 | 3.83 | 4 | 925 | 916 | 902 | 977 |
| PC 34:5 | 752.49 | 3.76 | 0 | 57 | 56 | 47 | 37 |
| PC 34:4 | 754.55 | 3.93 | 1 | 2 | 4 | 168 | 221 |
| PC 34:3 | 756.56 | 3.92 | 4 | 12 | 11 | 1280 | 1670 |
| PC 34:2 | 758.58 | 3.89 | 12 | 14 | 10 | 70395 | 94527 |
| PC 34:0/36:6p/36:7e | 762.61 | 3.83 | 11 | 273781 | 263598 | 218540 | 230349 |
| PC 35:5/36:4p/36:5e | 766.58 | 4.13 | 0 | 1 | 2 | 4 | 6 |
| PC 35:1/36:0p/36:1e | 774.57 | 4.23 | 2 | 33 | 39 | 117 | 161 |
| PC 35:0/36:0e | 776.62 | 3.83 | 4 | 3476 | 3397 | 2896 | 2876 |
| PC 36:6 | 778.55 | 3.98 | 1 | 117 | 113 | 260 | 297 |
| PC 36:5 | 780.56 | 3.95 | 4 | 45 | 34 | 3552 | 4437 |
| PC 36:4 | 782.58 | 3.92 | 1 | 8 | 7 | 25451 | 34306 |
| PC 36:3 | 784.59 | 3.91 | 1 | 6859 | 6778 | 23985 | 29047 |
| PC 36:2/38:8p/38:9e | 786.61 | 3.88 | 10 | 902 | 774 | 39897 | 52463 |
| PC 36:0/38:6p/38:7e | 790.64 | 3.83 | 21 | 1011329 | 990992 | 792715 | 849723 |
| PC 37:3/38:2p/38:3e | 798.57 | 4.28 | 1 | 11 | 26 | 796 | 1152 |
| PC 37:2/38:1p/38:2e | 800.58 | 3.88 | 11 | 1038 | 1053 | 1278 | 1273 |
| PC 37:1/38:0p/38:1e | 802.60 | 4.23 | 2 | 210 | 180 | 245 | 148 |
| PC 37:0/38:0e | 804.57 | 3.93 | 6 | 2024 | 1903 | 3291 | 3310 |
| PC 38:6 | 806.58 | 3.96 | 3 | 324 | 303 | 7779 | 11760 |
| PC 38:5 | 808.59 | 3.95 | 2 | 395 | 369 | 8299 | 12033 |
| PC 38:4 | 810.61 | 3.92 | 38 | 28438 | 27707 | 45181 | 50682 |
| PC 38:0/40:6p/40:7e | 818.66 | 3.83 | 11 | 4137 | 4031 | 4122 | 4515 |
| PC 40:4p/40:6e | 820.55 | 4.27 | 1 | 2 | 2 | 44 | 26 |
| PC 40:4p/40:5e | 822.62 | 3.93 | 9 | 276 | 264 | 1094 | 1243 |
| PC 40:2p/40:3e | 826.61 | 4.28 | 2 | 0 | 1 | 320 | 450 |
| PC 40:6 | 834.61 | 3.96 | 5 | 112 | 101 | 5484 | 8033 |
| PC 40:2/42:8p/42:9e | 842.61 | 4.03 | 0 | 0 | 1 | 2 | 15 |
| PC 40:1/42:7p/42:8e | 844.55 | 3.96 | 1 | 4 | 4 | 132 | 163 |
| PC 40:0/42:6p/42:7e | 846.69 | 3.83 | 24 | 1236 | 1221 | 1183 | 1261 |
| PC 42:5p/42:6e | 848.65 | 3.94 | 3 | 91 | 81 | 480 | 551 |
| PC 42:3p/43:4e | 852.69 | 3.90 | 3 | 2 | 0 | 28 | 37 |
| PC 42:2p/42:3e | 854.58 | 4.00 | 3 | 2 | 4 | 48 | 117 |
| PC 42:7/42:0e | 860.72 | 3.83 | 25 | 209 | 191 | 161 | 169 |
| PC 42:5 | 864.48 | 3.89 | 0 | 0 | 0 | 7 | 10 |
| PC 42:4/42:10p | 866.48 | 3.88 | 2 | 35 | 38 | 53 | 71 |
| PC 42:2/44:8p/44:9e | 870.51 | 3.82 | 0 | 55 | 51 | 49 | 56 |
| PC 42:1/44:7p/44:8e | 872.64 | 4.00 | 1 | 2 | 5 | 36 | 48 |
| PC 44:5p/44:6e | 876.70 | 3.93 | 3 | 68 | 70 | 401 | 550 |
| PC 44:7/44:0e | 888.75 | 3.84 | 22 | 2 | 2 | 4 | 2 |
| PC 44:6 | 890.48 | 3.92 | 0 | 0 | 0 | 8 | 12 |
| PC 44:5 | 892.51 | 3.90 | 0 | 0 | 0 | 6 | 9 |
| PC 44:4 | 894.51 | 3.89 | 0 | 1 | 1 | 5 | 4 |
| PC 44:3/46:9p/46:10e | 896.54 | 3.83 | 7 | 386 | 385 | 340 | 346 |
| PC 44:2/46:8p/46:9e | 898.54 | 3.83 | 6 | 413 | 437 | 361 | 393 |
| PC 44:1/46:7p/46:8e | 900.69 | 3.98 | 1 | 1 | 1 | 17 | 35 |
| PC 44:0/46:6p/46:7e | 902.71 | 3.95 | 0 | 0 | 0 | 16 | 25 |
| PC 46:5p/46:6e | 904.72 | 3.94 | 1 | 2 | 2 | 20 | 35 |
| PC 46:7/46:0e | 916.58 | 3.76 | 0 | 90 | 92 | 208 | 133 |
| PC std | 790.78 | 3.85 | 146331 | 19045 | 56398 | 13140 | 44410 |
| PE 30:0 | 664.43 | 4.75 | 0 | 3 | 0 | 5 | 2 |
| PE 30:1 | 662.41 | 4.88 | 0 | 23 | 21 | 3 | 5 |
| PE 34:0e | 706.77 | 4.95 | 1 | 24 | 24 | 3 | 4 |
| PE 36:2p/36:3e | 728.46 | 4.86 | 0 | 5 | 3 | 7 | 6 |
| PE 36:4p/36:5e | 724.42 | 4.94 | 0 | 26 | 21 | 2 | 4 |
| PE 38:0/40:6p/40:7e | 776.56 | 4.70 | 5 | 4 | 6 | 98 | 273 |
| PE 38:1/40:7p/40:8e | 774.55 | 4.74 | 1 | 4 | 1 | 29 | 95 |
| PE 38:2/40:8p/40:9e | 772.53 | 4.69 | 1 | 0 | 0 | 9 | 46 |
| PE 38:4 | 768.45 | 4.99 | 0 | 14 | 11 | 1 | 3 |
| PE 38:5 | 766.55 | 4.69 | 2 | 6 | 4 | 35 | 72 |
| PE 38:6 | 764.54 | 4.71 | 0 | 9 | 48 | 25 | 87 |
| PE 38:6p/38:7e | 750.55 | 4.68 | 8 | 1 | 8 | 183 | 389 |
| PE 40:1/42:7p/42:8e | 802.51 | 4.99 | 0 | 6 | 1 | 5 | 4 |
| PE 40:2p/40:3e | 784.59 | 5.23 | 0 | 2 | 64 | 5 | 74 |
| PE 40:4/42:10p | 796.54 | 4.73 | 0 | 3 | 0 | 2 | 10 |
| PE 40:4p/40:5e | 780.56 | 5.24 | 2 | 0 | 1 | 2 | 11 |
| PE 40:5 | 794.57 | 4.66 | 0 | 0 | 0 | 2 | 6 |
| PE 40:6 | 792.57 | 4.70 | 0 | 1 | 3 | 3 | 9 |
| PE 42:2/44:8p/44:9e | 828.59 | 4.78 | 0 | 8 | 42 | 4 | 44 |
| PE 42:2p/42:3e | 812.48 | 5.07 | 0 | 12 | 8 | 2 | 5 |
| PE 42:3p/42:4e | 810.60 | 4.89 | 3 | 1 | 2 | 4 | 5 |
| PE 42:4p/42:5e | 808.59 | 4.68 | 1 | 7 | 7 | 8 | 78 |
| PE 42:5p/42:6e | 806.58 | 4.79 | 0 | 0 | 1 | 0 | 1 |
| PE 42:8/42:0p/42:1e | 816.52 | 5.03 | 1 | 6 | 1 | 8 | 8 |
| PE 42:9/42:1p/42:2e | 814.69 | 4.77 | 2 | 0 | 1 | 0 | 4 |
| PE 44:0/46:6p/46:7e | 860.54 | 5.15 | 0 | 0 | 0 | 3 | 2 |
| PE 44:3p/44:4e | 838.56 | 4.79 | 0 | 11 | 9 | 1 | 0 |
| PE 44:8/44:0/44:1e | 844.57 | 4.79 | 0 | 23 | 21 | 2 | 5 |
| PE 46:0/48:6p/48:7e | 888.60 | 4.86 | 0 | 81 | 88 | 12 | 23 |
| PE 46:1/48:7p/48:8e | 886.62 | 4.72 | 30 | 0 | 15 | 1 | 14 |
| PE 46:3/48:9p/48:10e | 882.58 | 4.86 | 0 | 12 | 19 | 1 | 4 |
| PE 46:6 | 876.85 | 4.74 | 54 | 0 | 46 | 0 | 44 |
| PE 46:9/46:1p/46:2e | 870.79 | 4.71 | 99 | 0 | 15 | 1 | 11 |
| PE 48:2 | 912.61 | 4.90 | 0 | 20 | 14 | 2 | 2 |
| PE 48:6 | 904.58 | 5.14 | 0 | 5 | 0 | 1 | 0 |
| PE 48:7/48:0e | 902.94 | 4.89 | 0 | 75 | 67 | 6 | 9 |
| PE std | 748.53 | 4.70 | 23363 | 7 | 17432 | 6 | 13859 |
| SM 32:1 | 675.55 | 4.31 | 2 | 0 | 5 | 1221 | 1803 |
| SM 32:2 | 673.53 | 4.35 | 1 | 1 | 0 | 64 | 97 |
| SM 33:2 | 689.56 | 4.31 | 1 | 1 | 0 | 861 | 1238 |
| SM 34:1 | 703.58 | 4.30 | 5 | 1 | 2 | 29191 | 44752 |
| SM 34:2 | 701.56 | 4.33 | 8 | 3 | 5 | 2265 | 3772 |
| SM 35:0 | 719.57 | 4.68 | 1 | 1 | 1 | 32 | 134 |
| SM 35:1 | 717.59 | 4.30 | 3 | 1 | 1 | 531 | 797 |
| SM 35:2 | 715.58 | 4.33 | 1 | 0 | 1 | 49 | 78 |
| SM 36:1 | 731.46 | 4.36 | 1 | 77 | 59 | 105 | 64 |
| SM 36:2 | 729.59 | 4.33 | 3 | 1 | 0 | 1632 | 2684 |
| SM 38:3 | 755.58 | 4.54 | 2 | 0 | 2 | 1 | 28 |
| SM 38:4 | 753.59 | 4.30 | 10 | 5 | 11 | 1223 | 1311 |
| SM 38:5 | 751.58 | 4.34 | 0 | 1 | 1 | 412 | 503 |
| SM 39:0 | 775.49 | 4.43 | 1 | 104 | 75 | 181 | 108 |
| SM 39:4 | 767.59 | 4.29 | 1 | 1 | 0 | 109 | 118 |
| SM 39:5 | 765.59 | 4.33 | 2 | 6 | 5 | 32 | 46 |
| SM 40:0 | 789.50 | 4.47 | 0 | 1 | 1 | 4 | 2 |
| SM 41:0 | 803.61 | 4.38 | 1 | 40 | 26 | 101 | 104 |
| SM 41:1 | 801.69 | 4.28 | 9 | 294 | 257 | 3744 | 5798 |
| SM 41:2 | 799.67 | 4.31 | 3 | 2 | 6 | 1845 | 2784 |
| SM 41:3 | 797.65 | 4.35 | 0 | 0 | 0 | 208 | 298 |
| SM 41:4 | 795.64 | 4.28 | 3 | 3 | 2 | 376 | 419 |
| SM 41:6 | 791.95 | 4.61 | 0 | 13 | 10 | 1 | 1 |
| SM 42:2 | 813.69 | 4.32 | 39 | 302 | 654 | 17966 | 29873 |
| SM 42:3 | 811.67 | 4.35 | 9 | 201 | 318 | 5109 | 8498 |
| SM 43:0 | 831.64 | 4.37 | 3 | 1 | 1 | 138 | 198 |
| SM 43:1 | 829.69 | 4.53 | 13 | 0 | 12 | 6 | 101 |
| SM 43:2 | 827.70 | 4.31 | 8 | 42 | 126 | 606 | 947 |
| SM 43:4 | 823.67 | 4.28 | 7 | 6 | 5 | 1185 | 1341 |
| SM 43:5 | 821.66 | 4.32 | 2 | 4 | 3 | 480 | 544 |
| SM 43:6 | 819.52 | 4.49 | 1 | 94 | 69 | 191 | 117 |
| SM 44:1 | 843.73 | 4.28 | 14 | 1 | 13 | 13 | 54 |
| SM 44:2 | 841.53 | 4.53 | 0 | 99 | 56 | 191 | 87 |
| SM 44:5 | 835.67 | 4.32 | 82 | 11 | 99 | 4237 | 5488 |
| SM 44:6 | 833.66 | 4.35 | 7 | 3 | 15 | 1297 | 1941 |
| SM 46:5 | 863.55 | 4.57 | 1 | 93 | 58 | 189 | 101 |
| SM 46:7 | 859.67 | 4.28 | 2 | 0 | 1 | 13 | 14 |
| SM std | 734.77 | 4.29 | 46209 | 6 | 40032 | 4 | 36207 |
| TG 39:1 | 679.43 | 1.10 | 0 | 22 | 27 | 30 | 38 |
| TG 40:2 | 708.52 | 1.13 | 0 | 71 | 35 | 46 | 32 |
| TG 42:0 | 740.68 | 1.10 | 0 | 0 | 5 | 65 | 125 |
| TG 42:1 | 738.67 | 1.12 | 0 | 0 | 0 | 94 | 164 |
| TG 42:2 | 736.65 | 1.14 | 0 | 2 | 1 | 84 | 139 |
| TG 42:4 | 732.62 | 1.31 | 0 | 56 | 114 | 43 | 51 |
| TG 44:1 | 766.70 | 1.14 | 3 | 2 | 13 | 889 | 1340 |
| TG 44:2 | 764.68 | 1.15 | 1 | 0 | 1 | 428 | 518 |
| TG 44:3 | 762.67 | 1.16 | 0 | 0 | 0 | 108 | 117 |
| TG 45:1 | 780.72 | 1.15 | 2 | 1 | 10 | 141 | 202 |
| TG 45:2 | 778.56 | 1.13 | 0 | 214 | 113 | 130 | 107 |
| TG 45:3 | 776.69 | 1.16 | 0 | 0 | 0 | 17 | 18 |
| TG 46:1 | 794.73 | 1.16 | 15 | 12 | 63 | 3547 | 4096 |
| TG 46:2 | 792.71 | 1.17 | 1 | 1 | 10 | 1607 | 1700 |
| TG 46:3 | 790.70 | 1.18 | 0 | 0 | 1 | 445 | 476 |
| TG 46:4 | 788.68 | 1.19 | 0 | 0 | 2 | 116 | 123 |
| TG 46:5 | 786.67 | 1.19 | 0 | 0 | 1 | 13 | 12 |
| TG 47:0 | 810.58 | 1.13 | 0 | 29 | 10 | 18 | 13 |
| TG 47:1 | 808.74 | 1.17 | 11 | 6 | 25 | 545 | 588 |
| TG 47:2 | 806.73 | 1.17 | 0 | 5 | 10 | 217 | 215 |
| TG 47:5 | 800.69 | 1.20 | 0 | 0 | 0 | 19 | 16 |
| TG 48:1 | 822.76 | 1.17 | 12 | 24 | 80 | 10060 | 10820 |
| TG 48:2 | 820.74 | 1.18 | 3 | 4 | 12 | 5917 | 6593 |
| TG 48:3 | 818.73 | 1.19 | 0 | 1 | 0 | 2462 | 2840 |
| TG 48:4 | 816.71 | 1.20 | 0 | 4 | 1 | 931 | 967 |
| TG 48:5 | 814.70 | 1.20 | 1 | 1 | 16 | 194 | 207 |
| TG 49:1 | 836.78 | 1.18 | 4 | 12 | 20 | 1649 | 1815 |
| TG 50:2 | 848.73 | 1.38 | 1 | 9 | 9 | 13 | 1 |
| TG 50:3 | 846.76 | 1.20 | 5 | 13 | 14 | 13970 | 14530 |
| TG 50:4 | 844.75 | 1.21 | 9 | 12 | 8 | 5298 | 5610 |
| TG 50:5 | 842.73 | 1.22 | 19 | 9 | 45 | 1126 | 1286 |
| TG 52:2 | 876.98 | 1.20 | 1 | 0 | 0 | 152 | 110 |
| TG 52:3 | 874.79 | 1.48 | 13 | 0 | 7 | 3 | 79 |
| TG 52:4 | 872.95 | 1.23 | 7 | 0 | 7 | 84 | 219 |
| TG 53:0 | 894.76 | 1.27 | 22 | 11 | 19 | 4292 | 5626 |
| TG 53:1 | 892.75 | 1.45 | 0 | 11 | 5 | 11 | 6 |
| TG 53:4 | 886.79 | 1.23 | 3 | 370 | 49 | 1253 | 1567 |
| TG 54:0 | 908.86 | 1.29 | 0 | 158 | 386 | 134 | 338 |
| TG 54:1 | 906.80 | 1.24 | 6 | 360 | 529 | 3546 | 3999 |
| TG 54:4 | 900.77 | 1.20 | 1 | 1 | 0 | 31 | 10 |
| TG 54:5 | 898.79 | 1.24 | 43 | 11 | 66 | 27457 | 38015 |
| TG 54:6 | 896.78 | 1.26 | 105 | 6 | 81 | 14946 | 20253 |
| TG 55:2 | 918.75 | 1.29 | 7 | 2 | 2 | 952 | 1243 |
| TG 56:1 | 934.80 | 1.29 | 1 | 18 | 23 | 464 | 605 |
| TG 56:4 | 928.77 | 1.40 | 0 | 8 | 2 | 15 | 33 |
| TG 56:6 | 924.81 | 1.26 | 8 | 514 | 87 | 15906 | 21743 |
| TG 56:7 | 922.79 | 1.28 | 0 | 15 | 25 | 5807 | 8505 |
| TG 56:8 | 920.77 | 1.28 | 1 | 2 | 0 | 2533 | 3761 |
| TG 57:2 | 946.79 | 1.31 | 1 | 0 | 3 | 2350 | 3770 |
| TG 57:3 | 944.77 | 1.31 | 0 | 3 | 7 | 682 | 977 |
| TG std | 869.84 | 1.19 | 272218 | 64 | 137321 | 25988 | 165121 |
| FFA 16:0 | 257.24 | 1.66 | 0 | 0 | 0 | 1 | 0 |
| FFA 16:1 | 255.31 | 1.52 | 1 | 2 | 1 | 2 | 0 |
| FFA 18:1 | 283.35 | 1.54 | 2 | 31 | 33 | 19 | 3 |
| FFA 18:2 | 281.33 | 1.56 | 1 | 1 | 0 | 0 | 1 |
| FFA 18:3 | 279.23 | 1.60 | 0 | 92 | 111 | 468 | 532 |
| FFA 20:1 | 311.22 | 1.85 | 2 | 10 | 15 | 11 | 8 |
| FFA 20:3 | 307.26 | 1.61 | 0 | 9 | 11 | 36 | 53 |
| FFA 20:5 | 303.23 | 1.68 | 0 | 0 | 3 | 29 | 58 |
| FFA 22:0 | 341.26 | 1.78 | 0 | 4 | 4 | 2 | 3 |
| FFA 22:1 | 339.32 | 1.57 | 19 | 259 | 327 | 227 | 252 |
| FFA 22:2 | 337.31 | 1.61 | 0 | 6 | 11 | 14 | 8 |
| FFA 22:4 | 333.27 | 1.71 | 0 | 0 | 0 | 0 | 0 |
| FFA 22:5 | 331.26 | 1.67 | 0 | 1 | 1 | 2 | 6 |
| FFA 22:6 | 329.24 | 1.71 | 0 | 0 | 0 | 1 | 0 |
| FFA 24:0 | 369.30 | 1.80 | 0 | 11 | 22 | 9 | 9 |
| FFA 24:1 | 367.35 | 1.57 | 29 | 332 | 396 | 336 | 364 |
| FFA 24:2 | 365.34 | 1.62 | 0 | 2 | 2 | 13 | 27 |
| FFA 24:3 | 363.32 | 1.65 | 0 | 0 | 0 | 0 | 7 |
| FFA 24:4 | 361.23 | 1.60 | 0 | 0 | 0 | 0 | 1 |
| FFA 24:5 | 359.29 | 1.69 | 0 | 1 | 0 | 0 | 0 |
| FFA std | 286.42 | 1.50 | 33886 | 86 | 42274 | 116 | 24636 |

TABLE 7
Mass Spectrometry-based lipidomic analysis.
List of all ceramides identified in healthy human plasma and onto
the surface of HSPC:CHOL liposomes, as these were found
by LC-MS/MS. All samples were run in two technical replicates.
Abundance values are shown below for all ceramides identified.

| | | | | Corona- | Corona- | Average |
|---|---|---|---|---|---|---|
| | Bare | Bare | Average | coated | coated | Corona- |
| Compound | NPs 1 | NPs 2 | Bare NPs | NPs 1 | NPs 2 | coated NPs |
| CER(N(14)S(18)) | 0.005 | 0.004 | 0.0045 | 0.086 | 0.104 | 0.095 |
| CER(N(16)S(18)) | 0.047 | 0.043 | 0.045 | 1.207 | 1.274 | 1.2405 |
| CER(N(18)S(18)) | 0.016 | 0.008 | 0.012 | 0.367 | 0.365 | 0.366 |
| CER(N(20)S(18)) | 0 | 0 | 0 | 0.516 | 0.477 | 0.4965 |
| CER(N(22)S(18)) | 0.022 | 0.026 | 0.024 | 5.044 | 5.014 | 5.029 |
| CER(N(23)S(18)) | 0 | 0 | 0 | 4.92 | 4.726 | 4.823 |
| CER(N(24)S(18)) | 0.09 | 0.095 | 0.0925 | 20.65 | 19.72 | 20.185 |
| CER(N(26)S(18)) | 0.052 | 0.066 | 0.059 | 0.382 | 0.389 | 0.3855 |
| CER(N(24)S(16)) | 0.016 | 0.019 | 0.0175 | 0.928 | 0.872 | 0.9 |
| CER(N(24)S(17)) | 0.009 | 0.013 | 0.011 | 0.706 | 0.678 | 0.692 |
| CER(N(22)S(19)) | 0 | 0 | 0 | 0.228 | 0.249 | 0.2385 |
| CER(N(24)S(19)) | 0 | 0 | 0 | 1.211 | 1.252 | 1.2315 |
| CER(N(26)S(19)) | 0 | 0 | 0 | 0.032 | 0.024 | 0.028 |
| CER(N(23)S(20)) | 0 | 0 | 0 | 0.04 | 0.05 | 0.045 |
| CER(N(24)S(20)) | 0.018 | 0.02 | 0.019 | 0.233 | 0.208 | 0.2205 |
| CER(N(25)S(20)) | 0 | 0 | 0 | 0.021 | 0.022 | 0.0215 |
| CER(N(24)S(22)) | 0 | 0 | 0 | 0.019 | 0.022 | 0.0205 |
| CER(N(16)DS(18)) | 0 | 0 | 0 | 0.546 | 0.571 | 0.5585 |
| CER(N(18)DS(18)) | 0 | 0 | 0 | 0.509 | 0.529 | 0.519 |
| CER(N(22)DS(18)) | 0 | 0 | 0 | 1.422 | 1.843 | 1.6325 |
| CER(N(25)DS(18)) | 0 | 0 | 0 | 0.108 | 0.152 | 0.13 |
| CER(N(24)DS(19)) | 0 | 0 | 0 | 0.202 | 0.202 | 0.202 |
| CER(N(24)DS(20)) | 0.14 | 0.168 | 0.154 | 0.238 | 0.226 | 0.232 |
| CER(N(18)DS(24)) | 0 | 0 | 0 | 1.656 | 1.572 | 1.614 |
| CER(N(20)DS(24)) | 0.074 | 0.084 | 0.079 | 0.121 | 0.135 | 0.128 |
| CER(A(18)S(18)) | 0.738 | 0.766 | 0.752 | 0.892 | 0.986 | 0.939 |
| CER(A(20)S(18)) | 0 | 0 | 0 | 0.137 | 0.136 | 0.1365 |
| CER(A(22)S(18)) | 0 | 0 | 0 | 0.319 | 0.326 | 0.3225 |
| CER(A(18)DS(18)) | 0 | 0 | 0 | 0.221 | 0.221 | 0.221 |
| CER(A(20)DS(18)) | 1.001 | 1.001 | 1.001 | 1.062 | 1.017 | 1.0395 |
| CER(A(22)DS(18)) | 1.262 | 1.445 | 1.3535 | 1.515 | 1.503 | 1.509 |
| CER(A(24)DS(18)) | 0.602 | 0.808 | 0.705 | 0.821 | 0.827 | 0.824 |
| CER(A(24)H(16)) | 0 | 0 | 0 | 3.249 | 3.364 | 3.3065 |
| CER(A(25)H(16)) | 0 | 0 | 0 | 13.355 | 14.433 | 13.894 |
| CER(A(26)H(26)) | 0 | 0 | 0 | 0.983 | 1.028 | 1.0055 |
| CER(A(27)H(16)) | 0 | 0 | 0 | 0.25 | 0.224 | 0.237 |
| CER(A(25)H(18)) | 0 | 0 | 0 | 0 | 0 | 0 |

TABLE 8
Mass Spectrometry-based lipidomic analysis.
List of all oxylipins identified in healthy human plasma and onto the
surface of HSPC:CHOL liposomes, as these were found
by LC-MS/MS. All samples were run in two technical replicates.
Abundance values in pg/uL are shown below for all oxylipins identified.

| | | Bare | Bare | Average | Corona- | Corona- | Average |
|---|---|---|---|---|---|---|---|
| | Lipid | NPs | NPs | Bare | coated | coated | Corona- |
| Compound | Cocktail | 1 | 2 | NPs | NPs 1 | NPs 2 | coated NPs |
| 9(10) EpOME | 18 | 0 | 0 | 0 | 0 | 0 | 0 |
| 12(13) EpOME | 19.2 | 0 | 0 | 0 | 0 | 0 | 0 |
| 9,10 DiHOME | 18.1 | 0 | 0 | 0 | 0 | 0 | 0 |
| 12,13 DiHOME | 22 | 0.6 | 0.7 | 0.65 | 0.5 | 0.8 | 0.65 |
| 17,18-DiHETE | 21.7 | 0 | 0 | 0 | 0 | 0 | 0 |
| 5,15 DiHETE | 20.6 | 0 | 0 | 0 | 2 | 1.6 | 1.8 |
| 8,15 DiHETE | 20.7 | 0 | 0 | 0 | 2.5 | 2.4 | 2.45 |
| 11,12 DHET | 19.3 | 0 | 0 | 0 | 0 | 0 | 0 |
| 14,15 DHET | 17.8 | 0 | 0 | 0 | 0 | 0 | 0 |
| 9 HOTrE | 18.4 | 0 | 0 | 0 | 0.6 | 0.8 | 0.7 |
| 13 HOTrE | 22 | 0 | 0 | 0 | 1.8 | 1.6 | 1.7 |
| 9 HODE | 21.2 | 0 | 0 | 0 | 64.5 | 75.8 | 70.15 |
| 13 HODE | 20.7 | 2.9 | 3.3 | 3.1 | 53.5 | 61.3 | 57.4 |
| 5 HEPE | 22 | 0 | 0 | 0 | 2.2 | 1.6 | 1.9 |
| 8 HEPE | 21.3 | 0 | 0 | 0 | 0.7 | 1 | 0.85 |
| 11 HEPE | 22.4 | 0 | 0 | 0 | 1.2 | 1.4 | 1.3 |
| 12 HEPE | 22.5 | 0 | 0 | 0 | 0.6 | 0.8 | 0.7 |
| 18 HEPE | 21.8 | 0 | 0 | 0 | 3 | 3 | 3 |
| 5-oxo-ETE | 15 | 0 | 0 | 0 | 16.9 | 14.8 | 15.85 |
| 15-oxo-ETE | 17.4 | 0 | 0 | 0 | 25.2 | 21.4 | 23.3 |
| 5 HETE | 17 | 0 | 0 | 0 | 34 | 41.1 | 37.55 |
| 8 HETE | 18.1 | 0 | 0 | 0 | 8.2 | 8.3 | 8.25 |
| 9 HETE | 17.8 | 0 | 0 | 0 | 10.9 | 12.5 | 11.7 |
| 11 HETE | 21.8 | 0 | 0 | 0 | 20.1 | 20.6 | 20.35 |
| 12 HETE | 15.8 | 0 | 0 | 0 | 10.9 | 10.7 | 10.8 |
| 15 HETE | 24.1 | 0 | 0 | 0 | 18.2 | 21.1 | 19.65 |
| 20 HETE | 24 | 0 | 0 | 0 | 0 | 0 | 0 |
| 15 HETrE | 19.5 | 0 | 0 | 0 | 3.7 | 4.1 | 3.9 |
| LTB4 | 25.8 | 0 | 0 | 0 | 4.2 | 5 | 4.6 |
| 4 HDHA | 19.7 | 0 | 0 | 0 | 2.1 | 2.2 | 2.15 |
| 8-HDHA | 17.3 | 0 | 0 | 0 | 2.4 | 2.8 | 2.6 |
| 10 HDHA | 20.9 | 0 | 0 | 0 | 2.1 | 2 | 2.05 |
| 13 HDHA | 22.5 | 0 | 0 | 0 | 3.1 | 4 | 3.55 |
| 14 HDHA | 21.5 | 0 | 0 | 0 | 3.1 | 2.9 | 3 |
| 17 HDHA | 23.6 | 0 | 0 | 0 | 6.8 | 7.8 | 7.3 |
| 20 HDHA | 26 | 0 | 0 | 0 | 5.3 | 5.3 | 5.3 |
| LXA4 | 21.7 | 0 | 0 | 0 | 2.8 | 3.1 | 2.95 |
| 13,14 DiHDPA | 23 | 0 | 0 | 0 | 0 | 0 | 0 |
| 16,17 DIHDPA | 18.9 | 0 | 0 | 0 | 0 | 0 | 0 |
| 19,20 DiHDPA | 16.6 | 0 | 0 | 0 | 0 | 0 | 0 |
| 9 OxoODE | 16 | 1.5 | 1.2 | 1.35 | 10.8 | 8.9 | 9.85 |
| 13 OxoODE | 19.9 | 0 | 0 | 0 | 333.9 | 333.5 | 333.7 |
| Trans EKODE | 28.3 | 0.9 | 0.6 | 0.75 | 6 | 4.6 | 5.3 |
| PDX (10(S) 17(S | 23.3 | 0.4 | 0.7 | 0.55 | 1.3 | 1.7 | 1.5 |
| DIHDPA) | | 23.3 | | | | | |

### REFERENCES

1 Hadjidemetriou and Kostarelos, Nat. Nanotechnol., 2017, 12, 288-290
2 Dai et al. Adv. Healthc. Mater., 2018, 7, 1700575
3 Chen et al. Nanoscale, 2019, 11, 8760-8775
4 Debayle et al. Biomaterials, 2019, 219, 119357
5 Müller et al. Biomaterials, 2017, 115, 1-8
6 Docter et al. Nanomedicine, 2015, 10, 503-519
8 Hadjidemetriou et al. Biomaterials, 2019, 188, 118-129
9 Hadjidemetriou et al. Adv. Mater., 2019, 31, 1-9
10 Papafilippou et al., Nanoscale, 2020, 12, 10240-10253
11 Al-Ahmady et al. J. Control. Release, 2018, 276, 157-167
13 Hadjidemetriou et al. Nano Today, 2020, 34, 100901
14 Hadjidemetriou et al. ACS Nano, 2015, 9:8142-8156
15 Rasoulianboroujeni et al. Mater. Sci. Eng. C, 2017, 75, 191-197
36 Sioud and Sorensen, Biochem. Biophys. Res. Commun., 2003, 312, 1220-1225
42 K. Hamad-Schifferli, Nanomedicine, 2015, 10, 1663-1674
43 No et al., Anticancer Res., 2012, 32, 3467-71
44 Kamat et al., Cancer, 2010, 116, 1918-1925
45 Kowal et al., Proc. Natl. Acad. Sci. U.S.A, 2016, 113, E968-E977
47 Schwarzenbach et al. Nat. Rev. Cancer, 2011, 11, 426-437
48 Snyder et al., Cell, 2016, 164, 57-68
52 Dobrovolskaia et al., Nanomedicine Nanotechnology, Biol. Med., 2014, 10, 1453-1463
53 De Paoli et al., Biomaterials, 2014, 35, 6182-6194
54 Valpione et al., Eur. J. Cancer, 2018, 88, 1-9
55 Bagley et al., Clin. Cancer Res., 2020, 26, 397-407.
56 Hao et al., Br. J. Cancer, 2014, 111, 1482-1489
57 Fernandez-Garcia et al., Breast Cancer Res., 2019, 21, 149
58 Mizuno et al., Sci. Rep., 2019, 9, 1-11
59 Kuroi et al., Int. J. Oncol., 2001, 19, 143-148
60 Holdenrieder et al., in Annals of the New York Academy of Sciences, New York Academy of Sciences, 2008, vol. 1137, pp. 180-189
61 Kumar et al., Clin. Lung Cancer, 2010, 11, 36-44
62 de Miguel Perez et al. Sci. Rep., 2020, 10, 1-13
63 Liu et al., Ann. Oncol., 2020, 31, 745-759
64 Sina et al., Nat. Commun., 2018, 9, 1-13
65 Nguyen and Sim, Biosens. Bioelectron., 2015, 67, 443-449
66 Esteller, Nat. Rev. Genet., 2007, 8, 286-298
67 Kurdistani, Br. J. Cancer, 2007, 97, 1-5
68 Fraga and Esteller, Cell Cycle, 2005, 4, 1377-1381
69 Deligezer et al., Clin. Chem., 2008, 54, 1125-1131
70 Gezer et al., Oncol. Lett., 2012, 3, 1095-1098
71 Gezer et al., Int. J. Mol. Sci., 2015, 16, 29654-29662
73 Rago et al. Cancer Res., 2007, 67, 9364-9370

1. A method of identifying biomarkers from two or more distinct biomolecule classes in a biofluid, wherein the method comprises:
(a) contacting a plurality of nanoparticles with a biofluid to allow a biomolecule corona to form on the surface of said nanoparticles;
(b) isolating the nanoparticles and surface-bound biomolecule corona; and
(c) analyzing the biomolecule corona to identify biomarkers from two or more distinct biomarker classes.

2. The method according to claim 1, wherein step (a) is performed in vivo by administering a plurality of nanoparticles to a subject or in vitro using a biofluid

3. The method according to claim 2, wherein the nanoparticles are administered to a subject by intravenous injection.

4. The method according to claim 1, wherein the plurality of nanoparticles are incubated in the test biofluid sample in vitro under conditions to allow a biomolecule corona to form on the surface of said nanoparticles.

5. The method according to claim 1, wherein the analysis is conducted on a single biofluid sample.

6. The method according to claim 1, wherein the biofluid is a blood or blood fraction sample, optionally selected from serum or plasma.

7. The method according to claim 1, wherein at least one of the biomarker classes is selected from the group consisting of: protein, nucleic acid and lipid (or complexes of these).

8. The method according to claim 1, wherein the biomolecule corona is analyzed by two or more of proteomic, genomic and lipidomic analysis.

9. The method according to claim 1, wherein the biomolecule corona is analyzed by genomic analysis and at least one other biomarker class of analysis.

10. The method according to claim 9, wherein the biomolecule corona is analyzed by genomic analysis and proteomic and/or lipidomic and/or metabolomic analysis.

11. The method according to claim 1, wherein the nanoparticles are selected from liposomes, metallic nanoparticles (such as gold or silver), polymeric nanoparticles, fibre-shaped nanoparticles (such as carbon nanotubes and two dimensional nanoparticles such as graphene oxide nanoparticles; optionally wherein the nanoparticles are liposomes.

12. (canceled)

13. The method according to claim 11, wherein the nanoparticles are negatively charged.

14. The method according to claim 1, wherein the nanoparticles with surface-bound biomolecule corona are isolated from the biofluid and purified to remove unbound and highly abundant biomolecules to allow identification of low abundant biomarkers; optionally wherein the nanoparticles with surface-bound biomolecule corona are isolated from the biofluid and purified to remove unbound and highly abundant biomolecules by a method comprising size exclusion chromatography followed by ultrafiltration.

15. (canceled)

16. The method according to claim 1, wherein the biofluid sample analyzed is from a subject in a diseased state, such as cancer, optionally wherein the cancer is selected from the group consisting of: lung, melanoma or ovarian cancer.

17. The method according to claim 1, wherein one of the biomarker classes being analyzed is nucleic acid, such as DNA or RNA; optionally wherein the nucleic acid is cell-free DNA (cfDNA), optionally wherein the cfDNA is genomic DNA.

18. (canceled)

19. The method according to claim 17, wherein the amount or relative amount of total cell-free DNA (cfDNA) is determined.

20. The method according to claim 17, wherein a specific nucleic acid sequence within the cell-free nucleic acid is determined, optionally wherein the specific nucleic acid is indicative of a disease, such as being or comprising a disease-associated mutation.

21. The method according to claim 1, wherein a change in a biomarker in a biofluid from a subject in response to therapy is monitored; optionally wherein the therapy comprises administration of a drug molecule to the subject, optionally wherein the drug molecule is an anti-cancer compound.

22. (canceled)

23. A method for detecting a disease state in a subject, comprising:
(a) contacting a biofluid sample from the subject with a plurality of nanoparticles under conditions to allow a biomolecule corona to form on the surface of said nanoparticles; and
(b) analyzing the biomolecule corona for one or more disease-specific biomarkers from two or more biomolecule classes, which is determinative of the presence of a disease in said subject.

24. A method for monitoring cancer progression in a subject, comprising:
(a) contacting a biofluid sample from the subject with a plurality of nanoparticles under conditions to allow a biomolecule corona to form on the surface of said nanoparticles; and
(b) analyzing the biomolecule corona for one or more cancer-specific biomarkers from two or more biomolecule classes;
wherein the degree of cancer progression is determined based on the level of the cancer-specific biomarker(s) relative to a reference amount;
optionally wherein the cancer is selected from the group consisting of: ovarian, lung, prostate, melanoma and blood cancer, including leukemia, lymphoma and myeloma.

25. (canceled)
